(19) United States
(12) Patent Application Publication
(10) Pub. No.: US 2017/0125704 A1
(43) Pub. Date: May 4, 2017

(54) Organic Compound, Light-Emitting Element, Light-Emitting Device, Electronic Device, and Lighting Device

(71) Applicant: Semiconductor Energy Laboratory Co., Ltd., Kanagawa-ken (JP)

(72) Inventors: Hiroki Suzuki, Atsugi (JP); Sachiko KAWAKAMI, Atsugi (JP); Satoshi SEO, Sagamihara (JP); Tsunenori SUZUKI, Yokohama (JP); Naoaki Hashimoto, Sagamihara (JP); Yusuke TAKITA, Atsugi (JP)

(73) Assignee: Semiconductor Energy Laboratory Co., Ltd., Kanagawa-ken (JP)

(21) Appl. No.: 15/335,104

(22) Filed: Oct. 26, 2016

(30) Foreign Application Priority Data
Oct. 30, 2015 (JP) 2015-214211

(51) Int. Cl. H01L 51/00; C09K 11/02; C07D 209/80

(52) U.S. Cl. CPC H01L 51/0072; C07D 209/80; H01L 51/0052; C09K 11/025; H01L 51/5012

(57) ABSTRACT

Provided is a novel organic compound or a light-emitting element material. The organic compound includes an aromatic hydrocarbon group having 6 to 100 carbon atoms in the 5-position of benzo[a]carbazole and an aromatic hydrocarbon group having 6 to 30 carbon atoms in the 11-position thereof. The light-emitting element material includes the organic compound.

### BACKGROUND OF THE INVENTION

1. Field of the Invention

One embodiment of the present invention relates to an organic compound, and a light-emitting element, a display module, a lighting module, a display device, a light-emitting device, an electronic device, and a lighting device each including the organic compound. Note that one embodiment of the present invention is not limited to the above technical field. The technical field of one embodiment of the invention disclosed in this specification and the like relates to an object, a method, or a manufacturing method. In addition, one embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting device, a lighting device, a power storage device, a memory device, a method for driving any of them, and a method for manufacturing any of them.

2. Description of the Related Art

As next generation lighting devices or display devices, display devices using light-emitting elements (organic EL elements) in which organic compounds or organometallic complexes are used as light-emitting substances have been developed and reported because of their potential for thinness, lightness, high-speed response to input signals, low power consumption, application to flexible devices, and the like.

In an organic EL element, voltage application between electrodes, between which a light-emitting layer is interposed, causes recombination of electrons and holes injected from the electrodes, which brings a light-emitting substance into an excited state, and the return from the excited state to the ground state is accompanied by light emission. Since the spectrum of light emitted from a light-emitting substance depends on the light-emitting substance, use of different types of light-emitting substances makes it possible to obtain light-emitting elements which exhibit various colors.

Displays or lighting devices including organic EL elements can be suitably used for a variety of electronic devices as described above, and light emission mechanism, an element structure, and the like thereof are selected in accordance with applications or characteristics required. In the case where light emission with high efficiency is desired, an element emitting phosphorescence may be used, and in the case where reliability has priority, fluorescence may be used. In different light emission mechanism, different element structures and different materials are used, and the performance may depend on the positional relation of a level of an orbital or an excitation level. For this reason, it is preferable that the number of kinds of organic compounds that can be used as a light-emitting element material be large as much as possible.

In particular, reliability typified by a lifetime of a light-emitting element is naturally required because it is important for any electronic device and the most basic performance.

Patent Document 1 discloses an organic compound which can be used as a light-emitting element material and in which an aryl group having 30 or more carbon atoms is bonded to the 11-position of benzocarbazole.

### REFERENCE

### Patent Document

[Patent Document 1] United States Published Patent Application No. 2008/0122344

### SUMMARY OF THE INVENTION

An object of one embodiment of the present invention is to provide a novel organic compound. Another object is to provide an organic compound that can be used as a light emitting element material. Another object is to provide an organic compound that can be suitably used as a host material of a light-emitting element. Another object is to provide an organic compound that can be suitably used as a carrier-transport material of a light-emitting element. Another object is to provide a light-emitting element material. Another object is to provide a light-emitting element material that allows a light-emitting element to have a long lifetime. Another object is to provide a light-emitting element material that can be suitably used for a hole-transport layer of a blue fluorescent light-emitting element.

Another object of one embodiment of the present invention is to provide a novel light-emitting element. Another object of one embodiment of the present invention is to provide a light-emitting element with high reliability. Another object of one embodiment of the present invention is to provide a display module, a lighting module, a light-emitting device, a display device, an electronic device, and a lighting device each having high reliability.

It is only necessary that at least one of the above-described objects be achieved in one embodiment of the present invention. Note that the descriptions of these objects do not disturb the existence of other objects. One embodiment of the present invention does not necessarily have all the above objects. Other objects will be apparent from and can be derived from the description of the specification, the drawings, the claims, and the like.

One embodiment of the present invention is a light-emitting element material including an organic compound represented by General Formula (G1) shown below.

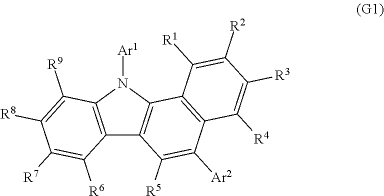

In General Formula (G1), Ar¹ represents a substituted or unsubstituted aromatic hydrocarbon group having 6 to 30 carbon atoms; Ar² represents a substituted or unsubstituted aromatic hydrocarbon group having 6 to 100 carbon atoms; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is a light-emitting element material including the organic compound represented by General Formula (G2) shown below.

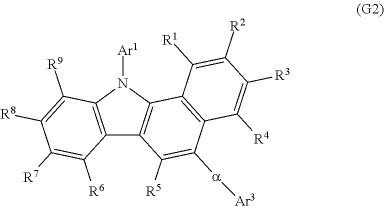

In General Formula (G2), Ar¹ represents a substituted or unsubstituted aromatic hydrocarbon group having 6 to 30 carbon atoms; α represents a substituted or unsubstituted divalent aromatic hydrocarbon group having 6 to 13 carbon atoms; Ar³ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is a light-emitting element material including an organic compound represented by General Formula (G3) shown below.

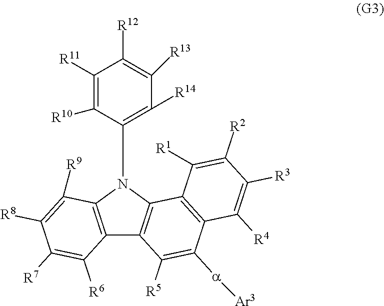

In General Formula (G3), α represents a substituted or unsubstituted divalent aromatic hydrocarbon group having 6 to 13 carbon atoms; Ar³ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton; each of R¹ to R⁵ and R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is a light-emitting element material including the organic compound represented by General Formula (G4) shown below.

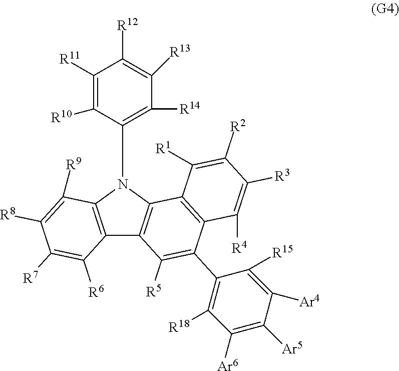

In General Formula (G4), any one of Ar⁴ to Ar⁶ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton; each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; each of R¹ to R⁵, R¹⁰ to R¹⁵, and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is any of the above-described light-emitting element materials in which R⁶ to R⁹ represent hydrogen.

Another embodiment of the present invention is a light-emitting element including any of the above-described light-emitting element materials.

Another embodiment of the present invention is an organic compound represented by General Formula (G1) shown below.

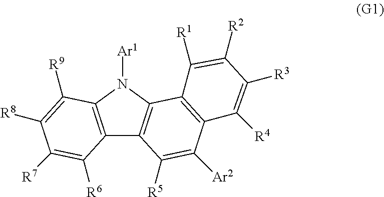

In General Formula (G1), Ar¹ represents a substituted or unsubstituted aromatic hydrocarbon group having 6 to 30 carbon atoms; Ar² represents a substituted or unsubstituted aromatic hydrocarbon group having 6 to 100 carbon atoms; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms. In the case where a fused polycyclic aromatic hydrocarbon skeleton is included in neither Ar¹ nor Ar², the total number of carbon atoms in Ar¹ and Ar² is greater than or equal to 19.

Another embodiment of the present invention is an organic compound represented by General Formula (G2) shown below.

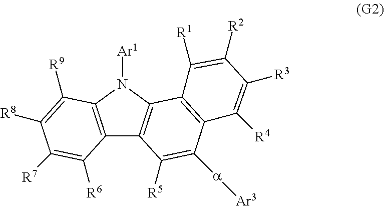

In General Formula (G2), Ar¹ represents a substituted or unsubstituted aromatic hydrocarbon group having 6 to 30 carbon atoms; α represents a substituted or unsubstituted divalent aromatic hydrocarbon group having 6 to 13 carbon atoms; Ar³ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms. When a fused polycyclic aromatic hydrocarbon skeleton is included in neither Ar¹, α, nor Ar³, the total number of carbon atoms in Ar¹, α, and Ar³ is greater than or equal to 19.

Another embodiment of the present invention is an organic compound represented by General Formula (G2) shown below.

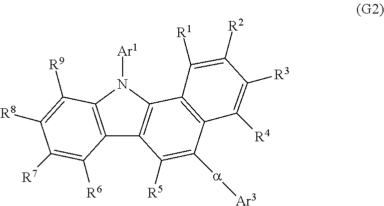

In General Formula (G2), Ar¹ represents a substituted or unsubstituted aromatic hydrocarbon group having 6 to 30 carbon atoms; α represents a substituted or unsubstituted divalent aromatic hydrocarbon group having 6 to 13 carbon atoms; Ar³ represents a substituted or unsubstituted aromatic hydrocarbon group having 10 to 30 carbon atoms and including a fused polycyclic (bi- to tetra-cyclic) aromatic hydrocarbon skeleton; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is an organic compound represented by General Formula (G3) shown below.

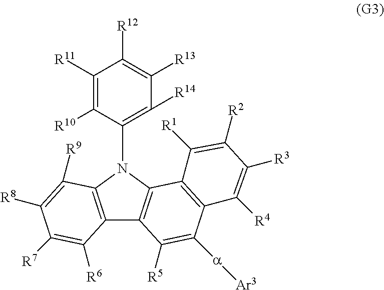

In General Formula (G3), α represents a substituted or unsubstituted divalent aromatic hydrocarbon group having 6 to 13 carbon atoms; Ar³ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton; each of R¹ to R⁵ and R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms. In the case where a fused polycyclic aromatic hydrocarbon skeleton is included in neither α nor Ar³, the total number of carbon atoms in α and Ar³ is greater than or equal to 13.

Another embodiment of the present invention is an organic compound represented by General Formula (G3) shown below.

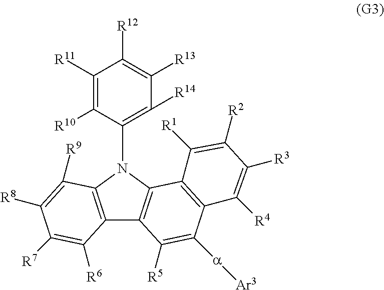

In General Formula (G3), α represents a substituted or unsubstituted divalent aromatic hydrocarbon group having 6 to 13 carbon atoms; Ar³ represents a substituted or unsubstituted aromatic hydrocarbon group having 10 to 30 carbon atoms and including a fused polycyclic (bi- to tetra-cyclic) aromatic hydrocarbon skeleton; each of R¹ to R⁵ and R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is an organic compound represented by General Formula (G4) shown below.

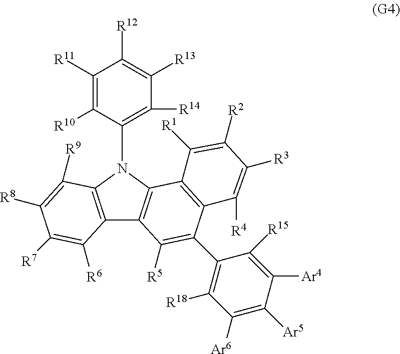

In General Formula (G4), any one of Ar⁴ to Ar⁶ represents a substituted or unsubstituted aromatic hydrocarbon group having 10 to 50 carbon atoms and including a monocyclic aromatic hydrocarbon skeleton or a fused polycyclic (bi- to dodeca-cyclic) aromatic hydrocarbon skeleton; each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; each of R¹ to R⁵, R¹⁰ to R¹⁵, and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is an organic compound represented by General Formula (G4) shown below.

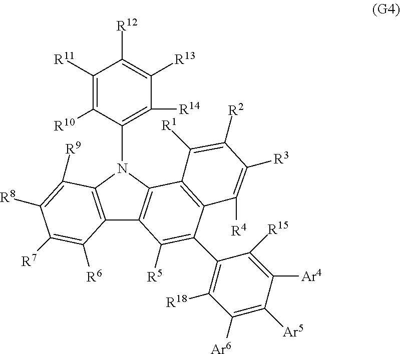

In General Formula (G4), any one of Ar⁴ to Ar⁶ represents a substituted or unsubstituted aromatic hydrocarbon group having 10 to 30 carbon atoms and including a fused polycyclic (bi- to tetra-cyclic) aromatic hydrocarbon skeleton; each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; each of R¹ to R⁵, R¹⁰ to R¹⁵, and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is the above-described organic compound in which the fused polycyclic aromatic hydrocarbon skeleton is any of a naphthalene skeleton, an anthracene skeleton, a phenanthrene skeleton, a fluorene skeleton, a pyrene skeleton, a tetracene skeleton, a tetraphene skeleton, a triphenylene skeleton, a chrysene skeleton, and a fluoranthene skeleton.

Another embodiment of the present invention is an organic compound represented by General Formula (G5) shown below.

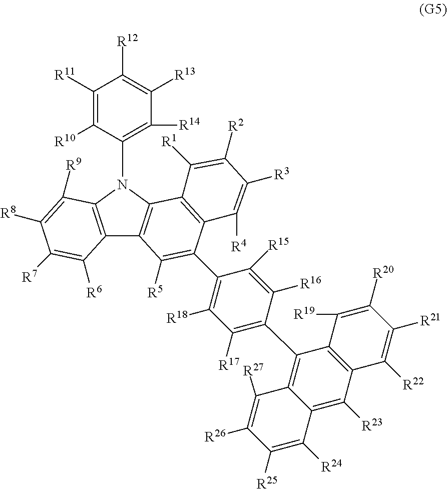

In General Formula (G5), each of R¹ to R⁵ and R¹⁰ to R²⁷ independently represents any of hydrogen, a saturated hydrocarbon group having 1 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is an organic compound represented by General Formula (G6) shown below.

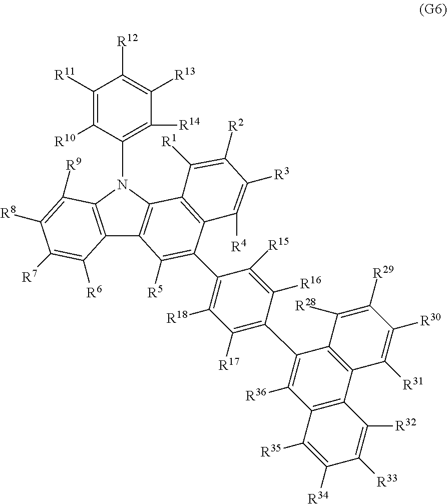

In General Formula (G6), each of R¹ to R⁵, R¹⁰ to R¹⁸, and R²⁸ to R³⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is an organic compound represented by General Formula (G7) shown below.

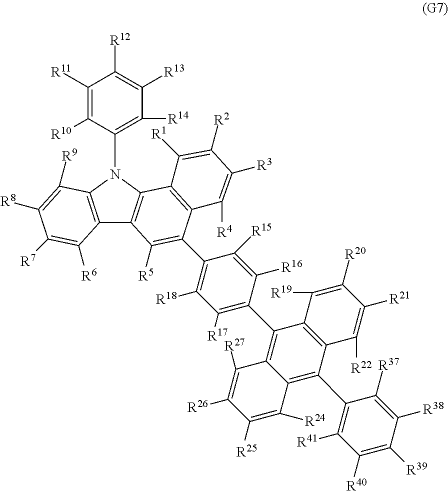

In General Formula (G7), each of R¹ to R⁵, R¹⁰ to R²², R²⁴ to R²⁷, and R³⁶ to R⁴⁰ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

Another embodiment of the present invention is any of the above-described organic compounds in which R⁶ to R⁹ represent hydrogen.

Another embodiment of the present invention is an organic compound represented by Structural Formula (100) shown below.

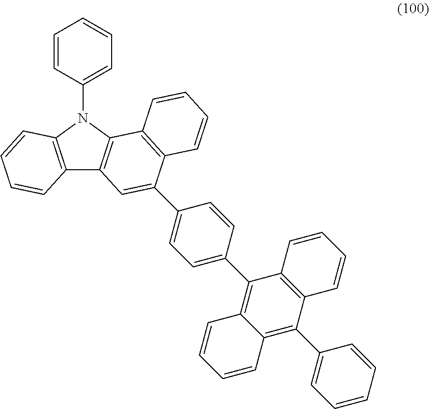 [SMILES: c1ccc(-c2c3ccccc3c(-c3ccc(-c4cc5c6ccccc6n(-c6ccccc6)c5c5ccccc45)cc3)c3ccccc23)cc1]

Another embodiment of the present invention is an organic compound represented by Structural Formula (126) shown below.

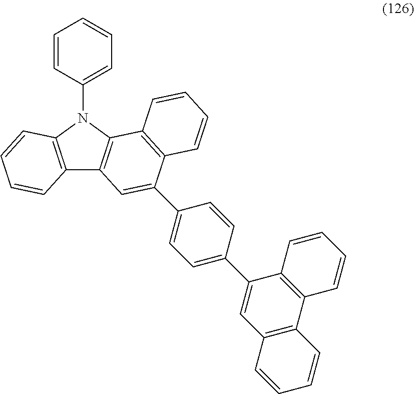 [SMILES: c1ccc(-n2c3ccccc3c3cc(-c4ccc(-c5cc6ccccc6c6ccccc56)cc4)c4ccccc4c32)cc1]

Another embodiment of the present invention is an organic compound represented by Structural Formula (136) shown below.

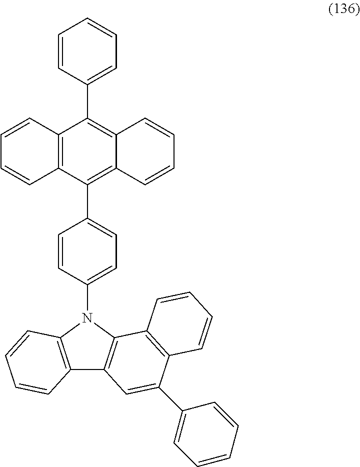 [SMILES: c1ccc(-c2c3ccccc3c(-c3ccc(-n4c5ccccc5c5cc(-c6ccccc6)c6ccccc6c54)cc3)c3ccccc23)cc1]

Another embodiment of the present invention is a light-emitting element material including any of the above-described organic compounds.

Another embodiment of the present invention is a light-emitting element including any of the above-described light-emitting element materials.

Another embodiment of the present invention is a light-emitting element that includes an organic compound including an anthracene skeleton and a carbazole skeleton and any of the above-described light-emitting element materials.

Another embodiment of the present invention is a light-emitting element including an anode, a cathode, and a layer provided between the anode and the cathode. The layer includes a first layer and a second layer. The first layer includes an organic compound including an anthracene skeleton and a carbazole skeleton. The second layer includes any of the above-described light-emitting element materials. The second layer is positioned between the first layer and the anode.

Another embodiment of the present invention is the above-described light-emitting element in which the first layer further includes a light-emitting substance.

Another embodiment of the present invention is a light-emitting device including any of the above-described light-emitting elements and at least one of a transistor and a substrate.

Another embodiment of the present invention is an electronic device including the above light-emitting device, and at least one of a sensor, an operation button, a speaker, and a microphone.

Another embodiment of the present invention is a lighting device including the above light-emitting device and a housing.

Note that the light-emitting device in this specification includes an image display device using a light-emitting element. The light-emitting device may be included in a module in which a light-emitting element is provided with a connector such as an anisotropic conductive film or a tape carrier package (TCP), a module in which a printed wiring board is provided at the end of a TCP, and a module in which an integrated circuit (IC) is directly mounted on a light-emitting element by a chip on glass (COG) method. The light-emitting device may be included in lighting equipment.

One embodiment of the present invention can provide a novel organic compound. One embodiment of the present invention can provide an organic compound that can be suitably used as a host material in a light-emitting layer of a light-emitting element. One embodiment of the present invention can provide an organic compound that can be suitably used as a light-emitting material of a light-emitting element. One embodiment of the present invention can provide an organic compound that can be suitably used as a carrier-transport material of a light-emitting element. One embodiment of the present invention can provide a light-emitting element material. One embodiment of the present invention can provide a light-emitting element material that allows a light-emitting element to have a long lifetime. One embodiment of the present invention can provide a light-emitting element material that can be suitably used as a material of a hole-transport layer of a blue fluorescent light-emitting element.

One embodiment of the present invention can provide a novel light-emitting element. One embodiment of the present invention can provide a display module, a lighting module, a light-emitting device, a display device, an electronic device, and a lighting device each having high reliability.

It is only necessary that at least one of the above effects be achieved in one embodiment of the present invention. Note that the description of these effects does not disturb the existence of other effects. One embodiment of the present invention does not necessarily achieve all the effects listed above. Other effects will be apparent from and can be derived from the description of the specification, the drawings, the claims, and the like.

### BRIEF DESCRIPTION OF THE DRAWINGS

FIGS. 1A to 1C are conceptual diagrams of light-emitting elements.

FIGS. 2A and 2B are conceptual diagrams of an active matrix light-emitting device.

FIGS. 3A and 3B are conceptual diagrams of an active matrix light-emitting device.

FIG. 4 is a conceptual diagram of an active matrix light-emitting device.

FIGS. 5A and 5B are conceptual diagrams of a passive matrix light-emitting device.

FIGS. 6A and 6B illustrate a lighting device.

FIGS. 7A to 7D illustrate electronic devices.

FIG. 8 illustrates a light source device.

FIG. 9 illustrates a lighting device.

FIG. 10 illustrates a lighting device.

FIG. 11 illustrates in-vehicle display devices and lighting devices.

FIGS. 12A to 12C illustrate an electronic device.

FIGS. 13A to 13C illustrate an electronic device.

FIGS. 14A and 14B show a ¹H NMR spectrum of PaBCPA.

FIG. 15 shows an absorption spectrum and an emission spectrum of a toluene solution of PaBCPA.

FIG. 16 shows an absorption spectrum and an emission spectrum of a thin film of PaBCPA.

FIGS. 17A and 17B show a ¹H NMR spectrum of aBCzPAP.

FIG. 18 shows an absorption spectrum and an emission spectrum of a toluene solution of aBCzPAP.

FIG. 19 shows an absorption spectrum and an emission spectrum of a thin film of aBCzPAP.

FIGS. 20A and 20B show a ¹H NMR spectrum of PaBCPPn.

FIG. 21 shows an absorption spectrum and an emission spectrum of a toluene solution of PaBCPPn.

FIG. 22 shows an absorption spectrum and an emission spectrum of a thin film of PaBCPPn.

FIG. 23 is a graph showing current density-luminance characteristics of a light-emitting element 1.

FIG. 24 is a graph showing luminance-current efficiency characteristics of the light-emitting element 1.

FIG. 25 is a graph showing voltage-luminance characteristics of the light-emitting element 1.

FIG. 26 is a graph showing voltage-current characteristics of the light-emitting element 1.

FIG. 27 is a graph showing luminance-external quantum efficiency characteristics of the light-emitting element 1.

FIG. 28 is a graph showing an emission spectrum of the light-emitting element 1.

FIG. 29 is a graph showing current density-luminance characteristics of light-emitting elements 2 and 3.

FIG. 30 is a graph showing luminance-current efficiency characteristics of the light-emitting elements 2 and 3.

FIG. 31 is a graph showing voltage-luminance characteristics of the light-emitting elements 2 and 3.

FIG. 32 is a graph showing voltage-current characteristics of the light-emitting elements 2 and 3.

FIG. 33 is a graph showing luminance-external quantum efficiency characteristics of the light-emitting elements 2 and 3.

FIG. 34 is a graph showing emission spectra of the light-emitting elements 2 and 3.

FIG. 35 is a graph showing time dependence of normalized luminance of the light-emitting elements 2 and 3.

FIG. 36 is a graph showing current density-luminance characteristics of a light-emitting element 4 and a light-emitting element 5.

FIG. 37 is a graph showing luminance-current efficiency characteristics of light-emitting elements 4 and 5.

FIG. 38 is a graph showing voltage-luminance characteristics of the light-emitting elements 4 and 5.

FIG. 39 is a graph showing voltage-current characteristics of the light-emitting elements 4 and 5.

FIG. 40 is a graph showing luminance-external quantum efficiency characteristics of the light-emitting elements 4 and 5.

FIG. 41 is a graph showing emission spectra of the light-emitting elements 4 and 5.

FIG. 42 is a graph showing time dependence of normalized luminance of the light-emitting elements 4 and 5.

FIG. 43 is a graph showing current density-luminance characteristics of a light-emitting element 6.

FIG. 44 is a graph showing luminance-current efficiency characteristics of the light-emitting element 6.

FIG. 45 is a graph showing voltage-luminance characteristics of the light-emitting element 6.

FIG. 46 is a graph showing voltage-current characteristics of the light-emitting element 6.

FIG. 47 is a graph showing luminance-external quantum efficiency characteristics of the light-emitting element 6.

FIG. 48 is a graph showing an emission spectrum of the light-emitting element 6.

FIG. 49 is a graph showing time dependence of normalized luminance of the light-emitting element 6.

### DETAILED DESCRIPTION OF THE INVENTION

An embodiment of the present invention will be explained below with reference to the drawings. Note that the present invention is not limited to the following description, and it will be readily appreciated by those skilled in the art that the mode and details can be changed in various different ways without departing from the spirit and the scope of the present invention. Accordingly, the present invention should not be interpreted as being limited to the content of the embodiment below.

In this specification, the specified number of carbon atoms of a group or a skeleton refers to the number of carbon atoms of the skeleton. In addition, the term “fused polycyclic aromatic hydrocarbon skeleton” refers to a skeleton of an aromatic hydrocarbon in which two or more rings are fused, and includes a naphthalene skeleton in its category.

An organic compound of one embodiment of the present invention includes an aromatic hydrocarbon group having 6 to 100 carbon atoms in the 5-position of benzo[a]carbazole and an aromatic hydrocarbon group having 6 to 30 carbon atoms in the 11-position of benzo[a]carbazole.

The organic compound has a high carrier-transport property and thus can be suitably used as a light-emitting element material such as a carrier-transport material or a host material of a light-emitting element. In particular, the organic compound is suitable as a carrier-transport material or a host material of a blue fluorescent element. Furthermore, the organic compound has a high hole-transport property and thus is preferably used as a material of a hole-transport layer. In the case where the organic compound includes a fused polycyclic aromatic hydrocarbon skeleton, the organic compound has a high electron-transport property in addition to a high hole-transport property and thus becomes a bipolar substance; thus, the organic compound can be suitably used as a host material for dispersing light-emitting substances in a light-emitting layer or a material of an electron-transport layer. Note that in this case, the fused polycyclic aromatic hydrocarbon skeleton is preferably an anthracene skeleton because the electron-transport property can be particularly high.

Furthermore, in a light-emitting element that uses a host material including an anthracene skeleton in a light-emitting layer, an organic compound of one embodiment of the present invention is used as a material of a hole-transport layer in contact with the light-emitting layer, in which case a hole is easily injected to the host material in the light-emitting layer and deterioration of the light-emitting element is not accelerated; thus, a light-emitting element with a long lifetime can be obtained. It is more preferable that the host material including an anthracene skeleton further include a carbazole skeleton because the above effect is more significant.

The organic compound of one embodiment of the present invention that is used as a light-emitting element material includes an aromatic hydrocarbon group having 6 to 100 carbon atoms in the 5-position which is highly reactive. Accordingly, the organic compound is a stable substance. A light-emitting element that includes the organic compound can have high reliability.

The organic compounds of embodiments of the present invention, which have such characteristics, can be represented by General Formula (G1) shown below.

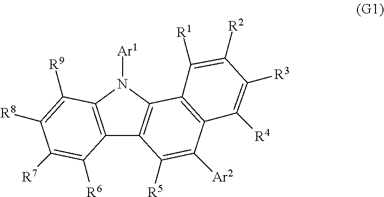

In General Formula (G1), Ar¹ represents a substituted or unsubstituted aromatic hydrocarbon group having 6 to 30 carbon atoms; and Ar² represents a substituted or unsubstituted aromatic hydrocarbon group having 6 to 100 carbon atoms.

A fused polycyclic aromatic hydrocarbon skeleton is preferably included in Ar¹ and/or Ar², in which case a light-emitting element with high heat resistance or high reliability is provided. In the case where a fused polycyclic aromatic hydrocarbon skeleton is included in neither Ar¹ nor Ar², the total number of carbon atoms in Ar¹ and Ar² is preferably 19 or more in order to provide a light-emitting element with high heat resistance or high reliability. Since a molecular structure including a fused polycyclic aromatic hydrocarbon skeleton can achieve a high carrier-transport property, a fused polycyclic aromatic hydrocarbon skeleton is preferably included.

In the organic compound represented by General Formula (G 1) shown above, Ar² preferably represents a group formed of a divalent aromatic hydrocarbon group and an aromatic hydrocarbon group including a monocyclic aromatic hydrocarbon skeleton or a fused polycyclic aromatic hydrocarbon skeleton in order to provide a light-emitting element with high heat resistance or high reliability. The organic compound can be represented by General Formula (G2) shown below.

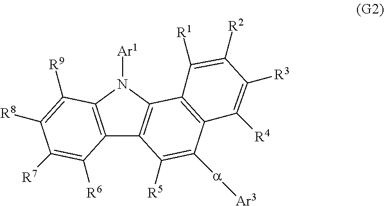

In General Formula (G2), Ar¹ has the same structure as Ar¹ in General Formula (G1).

In General Formula (G2), cc represents a substituted or unsubstituted divalent aromatic hydrocarbon group having 6 to 13 carbon atoms; and Ar³ represents a substituted or unsubstituted aromatic hydrocarbon group having 6 to 50 carbon atoms and including a monocyclic aromatic hydrocarbon skeleton or a fused polycyclic (bi- to dodeca-cyclic) aromatic hydrocarbon skeleton.

A fused polycyclic aromatic hydrocarbon skeleton is preferably included in Ar¹, α, and/or Ar³, in which case a light-emitting element with high heat resistance or high reliability is provided. In the case where a fused polycyclic aromatic hydrocarbon skeleton is included in neither Ar¹, α, nor Ar³, the total number of carbon atoms in Ar¹, α, and Ar³ is preferably 19 or more in order to provide a light-emitting element with high heat resistance or high reliability. Since a molecular structure including a fused polycyclic aromatic hydrocarbon skeleton can achieve a high carrier-transport property, a fused polycyclic aromatic hydrocarbon skeleton is preferably included.

In General Formula (G2), Ar¹ preferably represents a substituted or unsubstituted phenyl group in order to achieve a high carrier-transport property. That is, a preferable embodiment of the present invention is an organic compound represented by General Formula (G3) shown below.

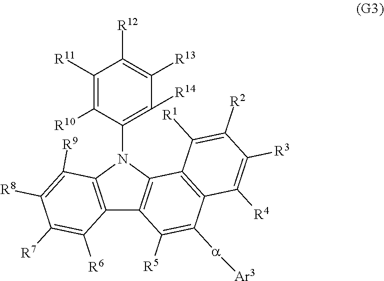

In General Formula (G3), a and Ar³ have the same structures as a and Ar³ in General Formula (G2).

A fused polycyclic aromatic hydrocarbon skeleton is preferably included in a and/or Ar³, in which case a light-emitting element with high heat resistance or high reliability is provided. In the case where a fused polycyclic aromatic hydrocarbon skeleton is included in neither α nor Ar³, the total number of carbon atoms in α and Ar³ is preferably 13 or more in order to provide a light-emitting element with high heat resistance or high reliability. Since a molecular structure including a fused polycyclic aromatic hydrocarbon skeleton can achieve a high carrier-transport property, a fused polycyclic aromatic hydrocarbon skeleton is preferably included.

In General Formulae (G2) and (G3), Ar³ preferably represents a substituted or unsubstituted aromatic hydrocarbon group having 10 to 30 carbon atoms and including a fused polycyclic (bi- to tetra-cyclic) aromatic hydrocarbon skeleton in terms of a cost for material synthesis or an increase in material purity.

In General Formula (G3), a preferably represents a substituted or unsubstituted phenylene group in terms of a cost for material synthesis or an increase in material purity. In that case, the group represented by Ar³ in General Formula (G3) is preferably bonded to the meta or para position of the phenylene group in order to achieve a light-emitting element with high reliability. That is, one embodiment of the present invention is an organic compound represented by General Formula (G4) shown below.

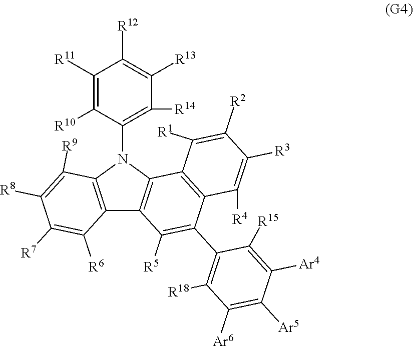

In General Formula (G4), any one of Ar⁴ to Ar⁶ represents a substituted or unsubstituted aromatic hydrocarbon group having 10 to 50 carbon atoms and including a monocyclic aromatic hydrocarbon skeleton or a fused polycyclic (bi- to dodeca-cyclic) aromatic hydrocarbon skeleton; each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

In General Formula (G4), in terms of a cost for material synthesis or an increase in material purity, it is preferable that any one of Ar⁴ to Ar⁶ represent a substituted or unsubstituted aromatic hydrocarbon group having 10 to 30 carbon atoms and including a fused polycyclic (bi- to tetra-cyclic) aromatic hydrocarbon skeleton, and that each of the other two of Ar⁴ to Ar⁶ independently represent any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

In General Formula (G4), Ar⁵ preferably represents a substituted or unsubstituted anthryl group in order to achieve a high carrier-transport property or high reliability. That is, one embodiment of the present invention is an organic compound represented by General Formula (G5).

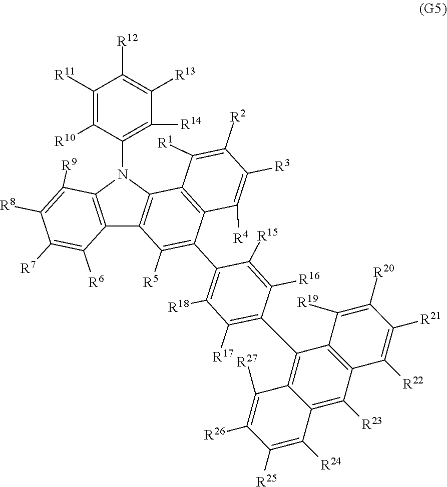

In the organic compound represented by General Formula (G5), R²³ preferably represents a substituted or unsubstituted phenyl group in order to provide a light-emitting element with high efficiency or high reliability. That is, one embodiment of the present invention is an organic compound represented by General Formula (G7) shown below.

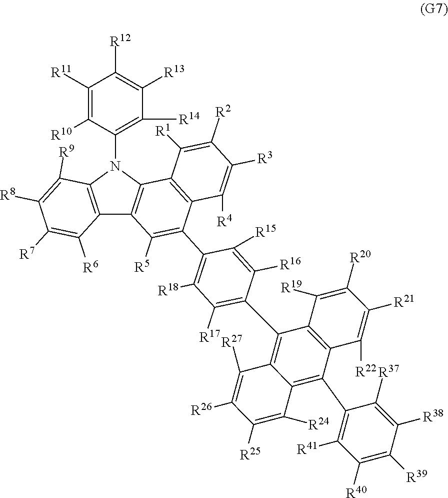

In General Formula (G4), Ar⁵ preferably represents a substituted or unsubstituted phenanthryl group in order to achieve a high level of phosphorescence. That is, one embodiment of the present invention is an organic compound represented by General Formula (G6) shown below.

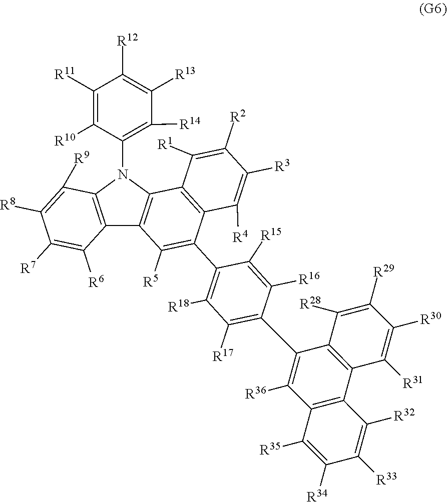

In General Formulae (G1) to (G7), each of R¹ to R⁵ and R¹⁰ to R³⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

In General Formulae (G1) to (G7), each of R⁶ to R⁹ independently represents any of hydrogen, a saturated hydrocarbon group having 1 to 6 carbon atoms, and a cyclic saturated hydrocarbon group having 3 to 6 carbon atoms. Note that an organic compound with a structure in which all of R⁶ to R⁹ represent hydrogen has a great advantage in a cost for material synthesis and also an advantage in an increase in material purity.

In General Formulae (G2), (G3), and (G4), in the case where Ar³ to Ar⁶ represent a substituted or unsubstituted aromatic hydrocarbon group having 10 to 30 carbon atoms and including a fused polycyclic (bi- to tetra-cyclic) aromatic hydrocarbon skeleton, the fused polycyclic aromatic hydrocarbon skeleton is preferably a naphthalene skeleton, an anthracene skeleton, a phenanthrene skeleton, a fluorene skeleton, a pyrene skeleton, a tetracene skeleton, a tetraphene skeleton, a triphenylene skeleton, a chrysene skeleton, or a fluoranthene skeleton.

In this specification, specific examples of a saturated hydrocarbon group having 1 to 6 carbon atoms include a methyl group, an ethyl group, a propyl group, an isopropyl group, an n-butyl group, a sec-butyl group, an isobutyl group, a tert-butyl group, an n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, and a branched or non-branched hexyl group.

Specific examples of a cyclic saturated hydrocarbon group having 3 to 6 carbon atoms include a cyclopropyl group, a cyclobutyl group, a cyclopentyl group, a cyclohexyl group, and the like.

Examples of an aromatic hydrocarbon group having 1 to 13 carbon atoms include a phenyl group, a biphenyl group, a naphthyl group, and a fluorenyl group. When the group is a fluorenyl group, the fluorenyl group preferably includes a substituent, and the fluorenyl group is preferably a 9,9-dimethylfluorenyl group or a 9,9-diphenylfluorenyl group. Note that the phenyl groups in the 9,9-diphenylfluorenyl group may be bonded to form a 9,9′-spirobifluorenyl group.

Examples of a divalent aromatic hydrocarbon group having 6 to 13 carbon atoms include a phenylene group, a biphenyl-diyl group, a naphthylene group, and a fluorene-diyl group. When the group is a fluorene-diyl group, the fluorene-diyl group preferably includes a substituent, and the fluorene-diyl group is preferably a 9,9-dimethylfluorene-diyl group or a 9,9-diphenylfluorene-diyl group. Note that the phenyl groups in the 9,9-diphenylfluorene-diyl group may be bonded to form a 9,9′-spirobifluorene-diyl group.

Note that in this specification, in the case where a group or a skeleton further includes a “substituent”, the substituent corresponds to a saturated hydrocarbon group having 1 to 6 carbon atoms, a cyclic saturated hydrocarbon group having 3 to 6 carbon atoms, or a phenyl group, for example.

Some specific examples of the organic compounds of embodiments of the present invention with the above structure are shown below.

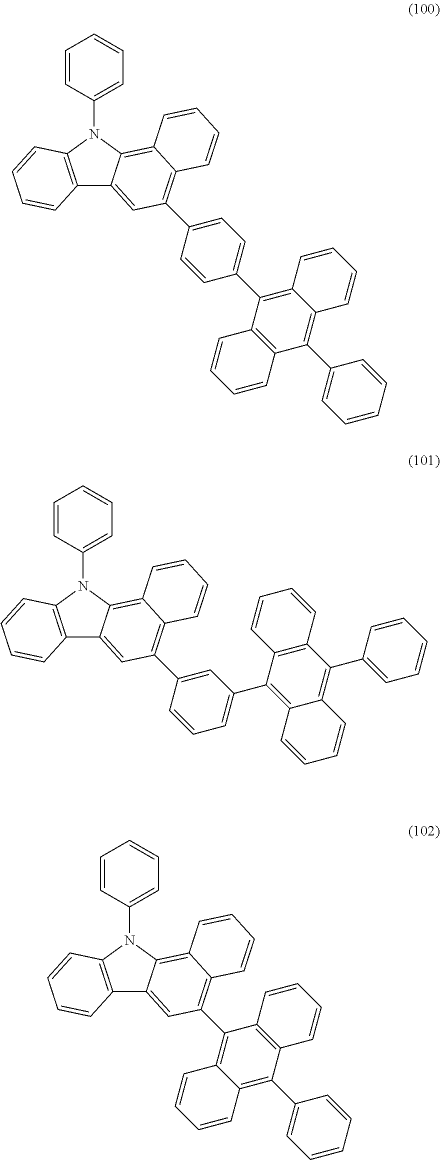 [SMILES: c1ccc(-c2c3ccccc3c(-c3ccc(-c4cc5c6ccccc6n(-c6ccccc6)c5c5ccccc45)cc3)c3ccccc23)cc1 ; c1ccc(-c2c3ccccc3c(-c3cccc(-c4cc5c6ccccc6n(-c6ccccc6)c5c5ccccc45)c3)c3ccccc23)cc1 ; c1ccc(-c2c3ccccc3c(-c3cc4c5ccccc5n(-c5ccccc5)c4c4ccccc34)c3ccccc23)cc1]

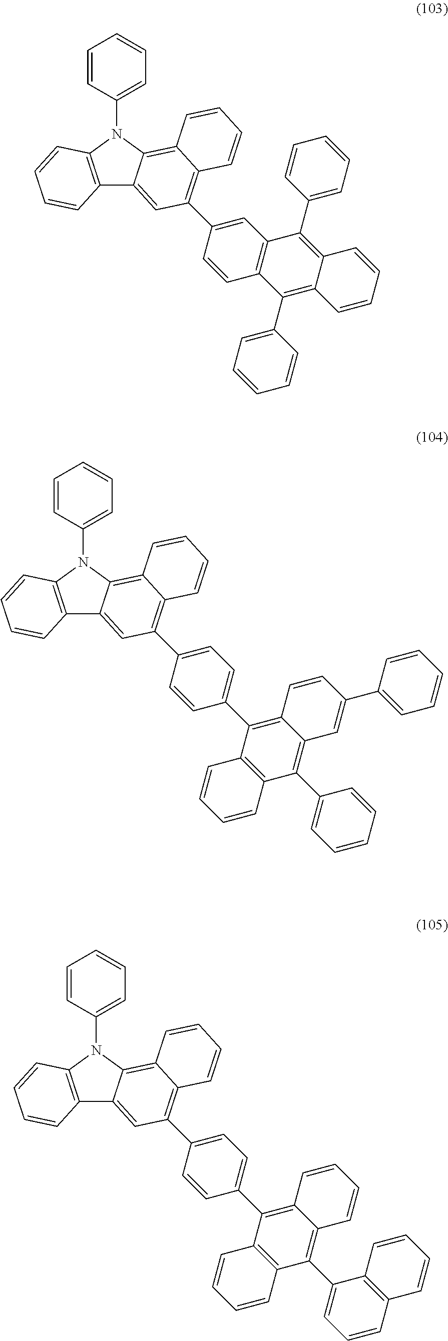 [SMILES: c1ccc(-c2c3ccccc3c(-c3ccccc3)c3cc(-c4cc5c6ccccc6n(-c6ccccc6)c5c5ccccc45)ccc23)cc1 ; c1ccc(-c2ccc3c(-c4ccc(-c5cc6c7ccccc7n(-c7ccccc7)c6c6ccccc56)cc4)c4ccccc4c(-c4ccccc4)c3c2)cc1 ; c1ccc(-n2c3ccccc3c3cc(-c4ccc(-c5c6ccccc6c(-c6cccc7ccccc67)c6ccccc56)cc4)c4ccccc4c32)cc1]

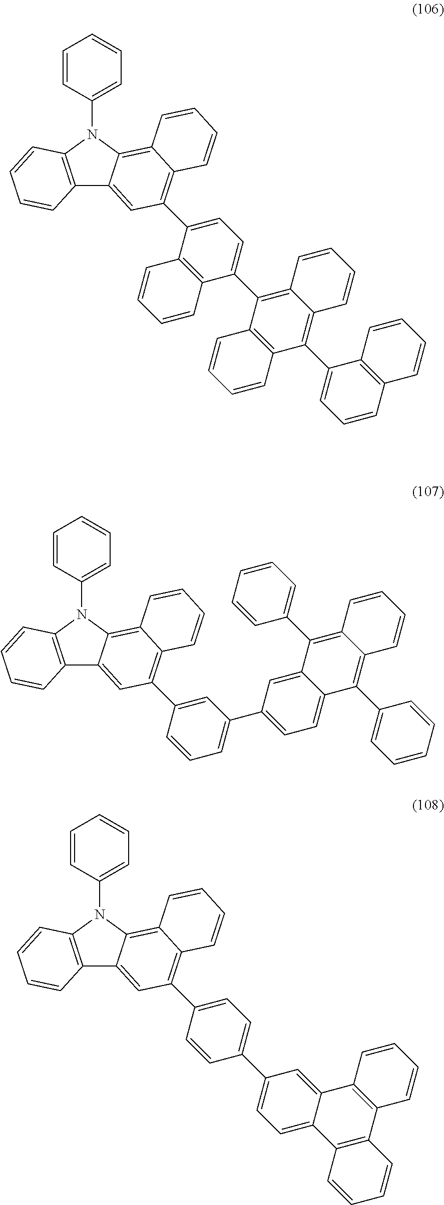 [SMILES: c1ccc(-n2c3ccccc3c3cc(-c4ccc(-c5c6ccccc6c(-c6cccc7ccccc67)c6ccccc56)c5ccccc45)c4ccccc4c32)cc1 ; c1ccc(-c2c3ccccc3c(-c3ccccc3)c3cc(-c4cccc(-c5cc6c7ccccc7n(-c7ccccc7)c6c6ccccc56)c4)ccc23)cc1 ; c1ccc(-n2c3ccccc3c3cc(-c4ccc(-c5ccc6c7ccccc7c7ccccc7c6c5)cc4)c4ccccc4c32)cc1]

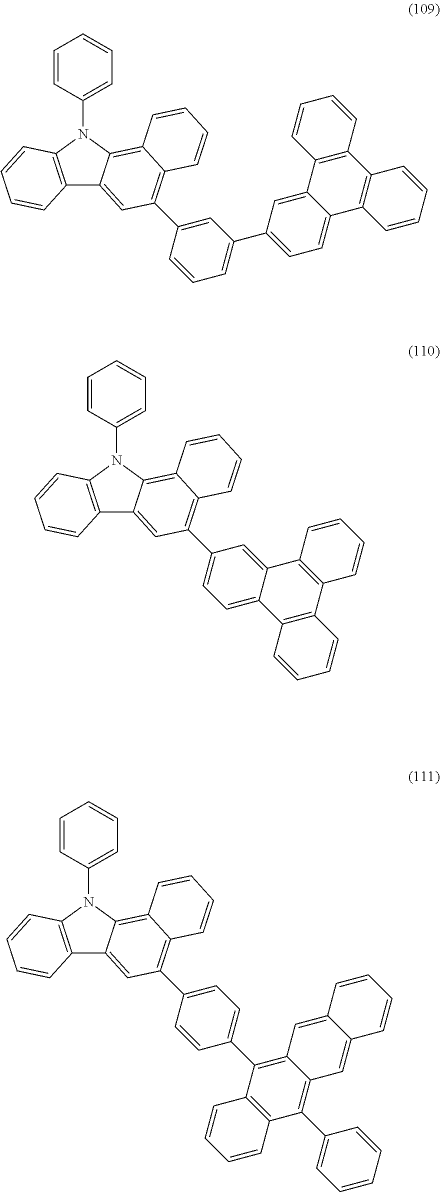 [SMILES: c1ccc(-n2c3ccccc3c3cc(-c4cccc(-c5ccc6c7ccccc7c7ccccc7c6c5)c4)c4ccccc4c32)cc1 ; c1ccc(-n2c3ccccc3c3cc(-c4ccc5c6ccccc6c6ccccc6c5c4)c4ccccc4c32)cc1 ; c1ccc(-c2c3ccccc3c(-c3ccc(-c4cc5c6ccccc6n(-c6ccccc6)c5c5ccccc45)cc3)c3cc4ccccc4cc23)cc1]

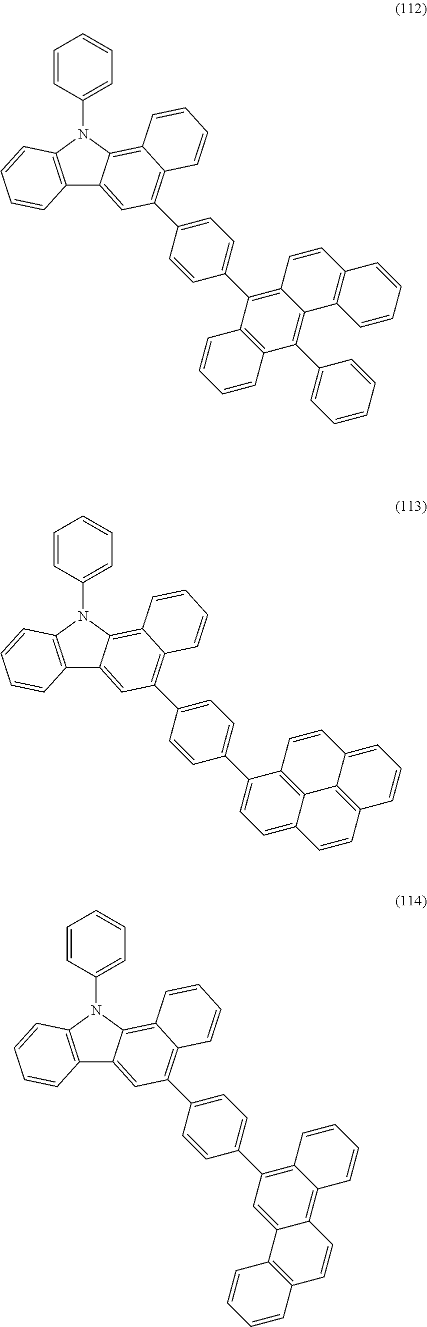 [SMILES: c1ccc(-c2c3ccccc3c(-c3ccc(-c4cc5c6ccccc6n(-c6ccccc6)c5c5ccccc45)cc3)c3ccc4ccccc4c23)cc1 ; c1ccc(-n2c3ccccc3c3cc(-c4ccc(-c5ccc6ccc7cccc8ccc5c6c78)cc4)c4ccccc4c32)cc1 ; c1ccc(-n2c3ccccc3c3cc(-c4ccc(-c5cc6c7ccccc7ccc6c6ccccc56)cc4)c4ccccc4c32)cc1]

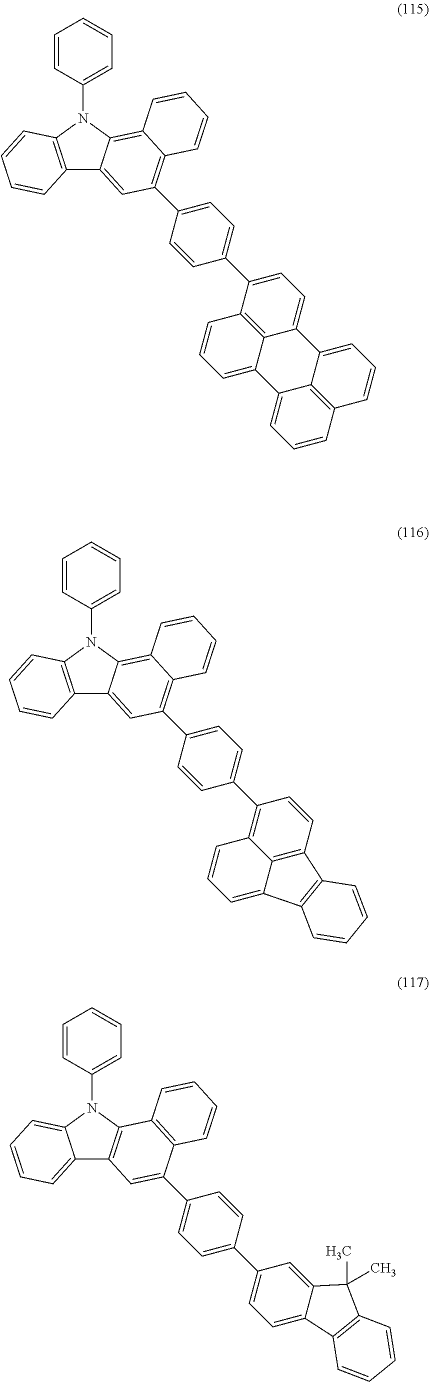 [SMILES: c1ccc(-n2c3ccccc3c3cc(-c4ccc(-c5ccc6c7cccc8cccc(c9cccc5c96)c87)cc4)c4ccccc4c32)cc1 ; c1ccc(-n2c3ccccc3c3cc(-c4ccc(-c5ccc6c7c(cccc57)-c5ccccc5-6)cc4)c4ccccc4c32)cc1 ; CC1(C)c2ccccc2-c2ccc(-c3ccc(-c4cc5c6ccccc6n(-c6ccccc6)c5c5ccccc45)cc3)cc21]

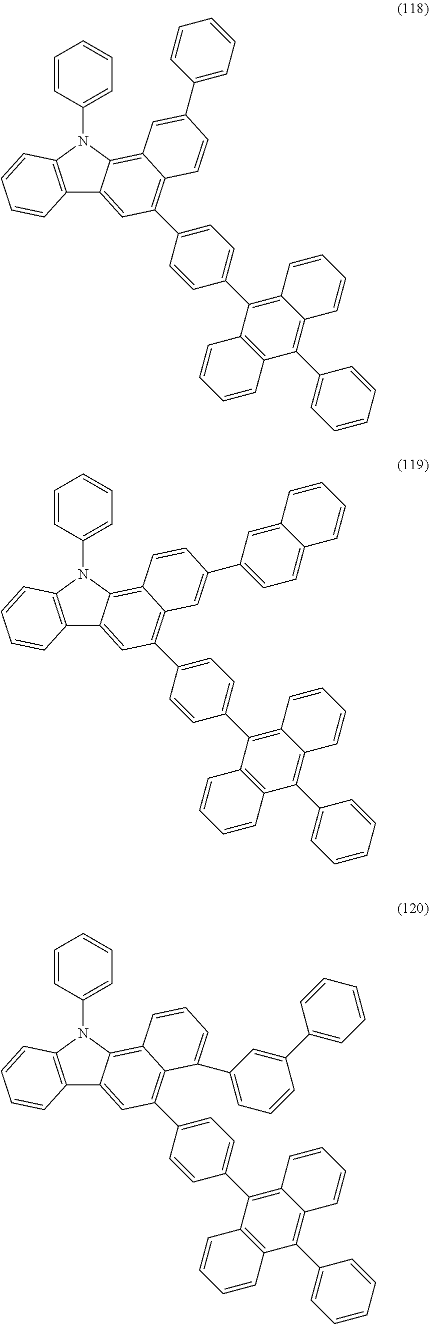 [SMILES: c1ccc(-c2ccc3c(-c4ccc(-c5c6ccccc6c(-c6ccccc6)c6ccccc56)cc4)cc4c5ccccc5n(-c5ccccc5)c4c3c2)cc1 ; c1ccc(-c2c3ccccc3c(-c3ccc(-c4cc5c6ccccc6n(-c6ccccc6)c5c5ccc(-c6ccc7ccccc7c6)cc45)cc3)c3ccccc23)cc1 ; c1ccc(-c2cccc(-c3cccc4c3c(-c3ccc(-c5c6ccccc6c(-c6ccccc6)c6ccccc56)cc3)cc3c5ccccc5n(-c5ccccc5)c43)c2)cc1]

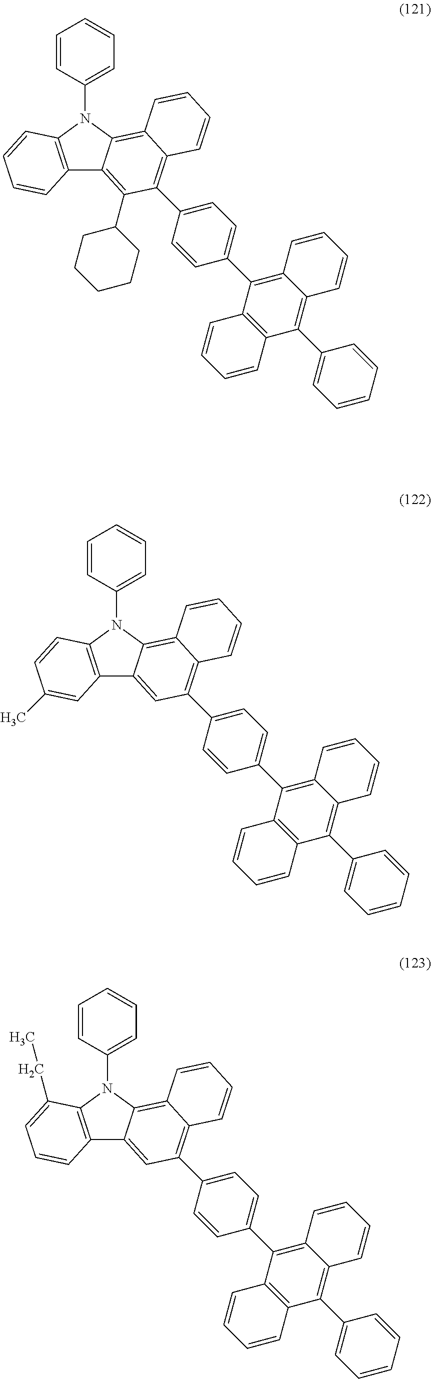 [SMILES: c1ccc(-c2c3ccccc3c(-c3ccc(-c4c(C5CCCCC5)c5c6ccccc6n(-c6ccccc6)c5c5ccccc45)cc3)c3ccccc23)cc1 ; Cc1ccc2c(c1)c1cc(-c3ccc(-c4c5ccccc5c(-c5ccccc5)c5ccccc45)cc3)c3ccccc3c1n2-c1ccccc1 ; CCc1cccc2c3cc(-c4ccc(-c5c6ccccc6c(-c6ccccc6)c6ccccc56)cc4)c4ccccc4c3n(-c3ccccc3)c12]

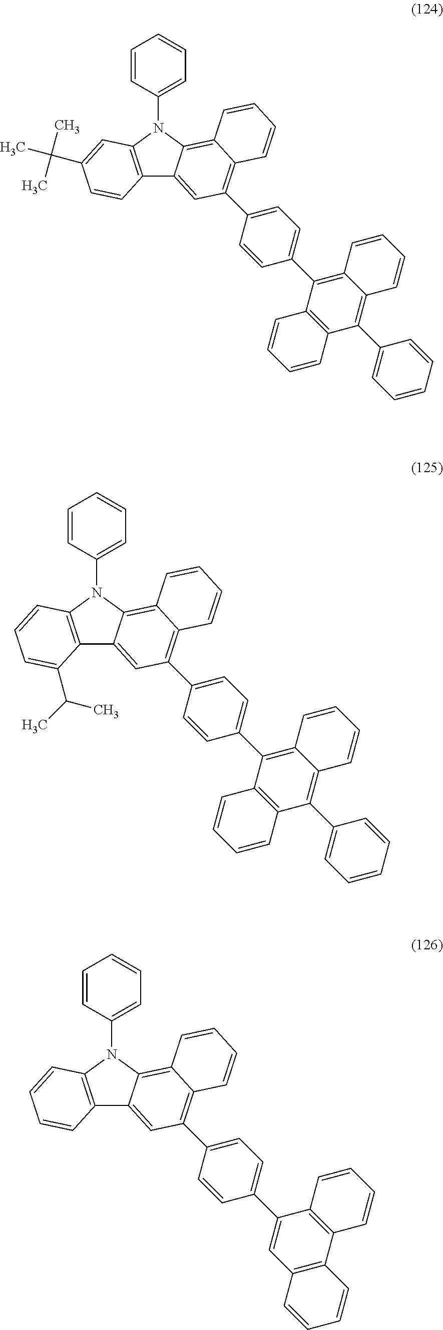 [SMILES: CC(C)(C)c1ccc2c3cc(-c4ccc(-c5c6ccccc6c(-c6ccccc6)c6ccccc56)cc4)c4ccccc4c3n(-c3ccccc3)c2c1 ; CC(C)c1cccc2c1c1cc(-c3ccc(-c4c5ccccc5c(-c5ccccc5)c5ccccc45)cc3)c3ccccc3c1n2-c1ccccc1 ; c1ccc(-n2c3ccccc3c3cc(-c4ccc(-c5cc6ccccc6c6ccccc56)cc4)c4ccccc4c32)cc1]

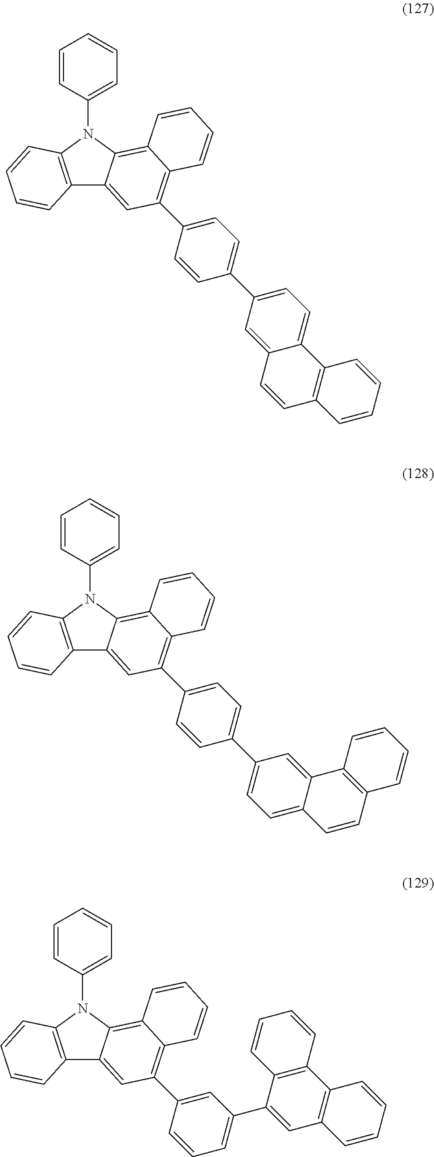 [SMILES: c1ccc(-n2c3ccccc3c3cc(-c4ccc(-c5ccc6c(ccc7ccccc76)c5)cc4)c4ccccc4c32)cc1 ; c1ccc(-n2c3ccccc3c3cc(-c4ccc(-c5ccc6ccc7ccccc7c6c5)cc4)c4ccccc4c32)cc1 ; c1ccc(-n2c3ccccc3c3cc(-c4cccc(-c5cc6ccccc6c6ccccc56)c4)c4ccccc4c32)cc1]

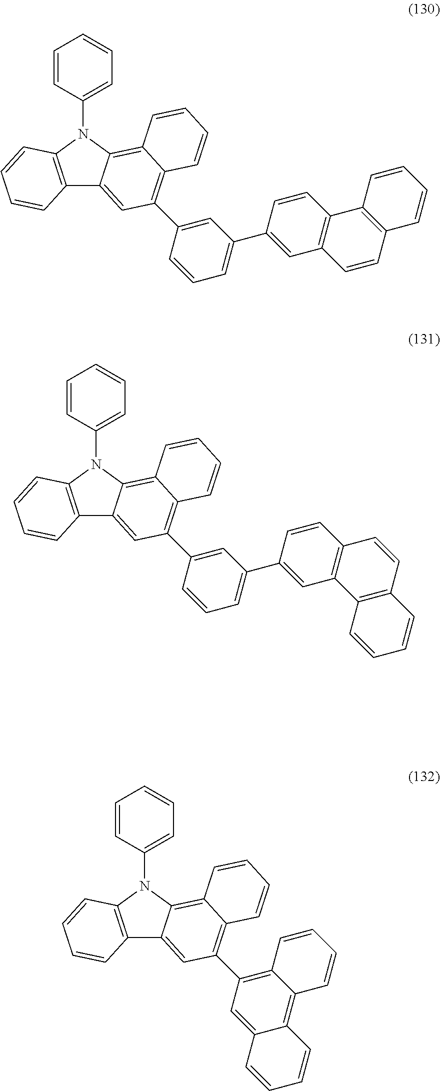 [SMILES: c1ccc(-n2c3ccccc3c3cc(-c4cccc(-c5ccc6c(ccc7ccccc76)c5)c4)c4ccccc4c32)cc1 ; c1ccc(-n2c3ccccc3c3cc(-c4cccc(-c5ccc6ccc7ccccc7c6c5)c4)c4ccccc4c32)cc1 ; c1ccc(-n2c3ccccc3c3cc(-c4cc5ccccc5c5ccccc45)c4ccccc4c32)cc1]

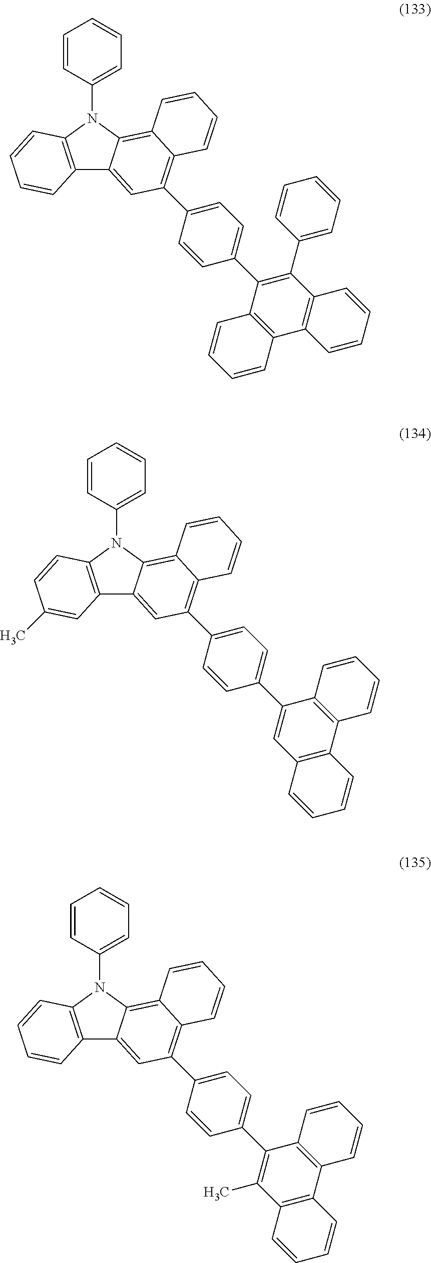 [SMILES: c1ccc(-c2c(-c3ccc(-c4cc5c6ccccc6n(-c6ccccc6)c5c5ccccc45)cc3)c3ccccc3c3ccccc23)cc1 ; Cc1ccc2c(c1)c1cc(-c3ccc(-c4cc5ccccc5c5ccccc45)cc3)c3ccccc3c1n2-c1ccccc1 ; Cc1c(-c2ccc(-c3cc4c5ccccc5n(-c5ccccc5)c4c4ccccc34)cc2)c2ccccc2c2ccccc12]

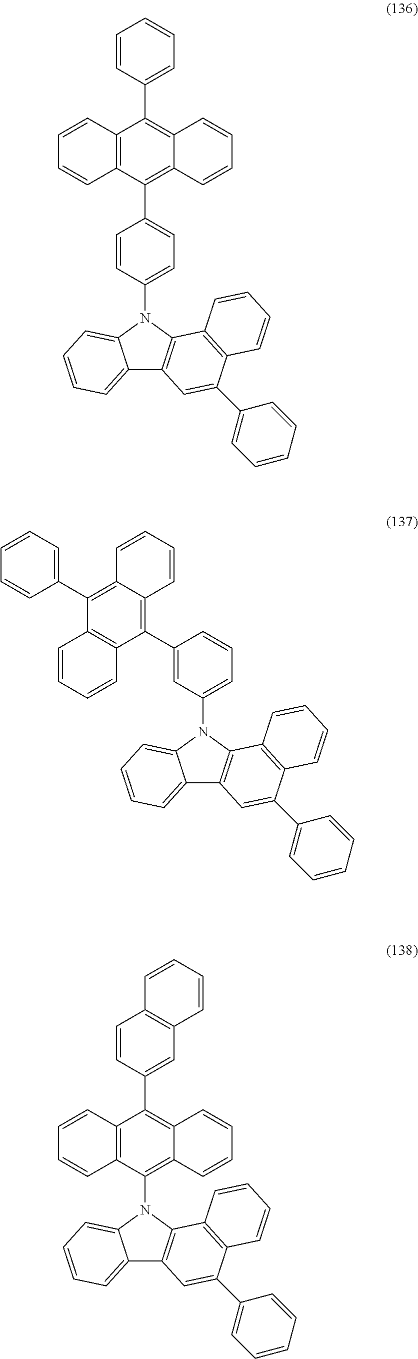 [SMILES: c1ccc(-c2c3ccccc3c(-c3ccc(-n4c5ccccc5c5cc(-c6ccccc6)c6ccccc6c54)cc3)c3ccccc23)cc1 ; c1ccc(-c2c3ccccc3c(-c3cccc(-n4c5ccccc5c5cc(-c6ccccc6)c6ccccc6c54)c3)c3ccccc23)cc1 ; c1ccc(-c2cc3c4ccccc4n(-c4c5ccccc5c(-c5ccc6ccccc6c5)c5ccccc45)c3c3ccccc23)cc1]

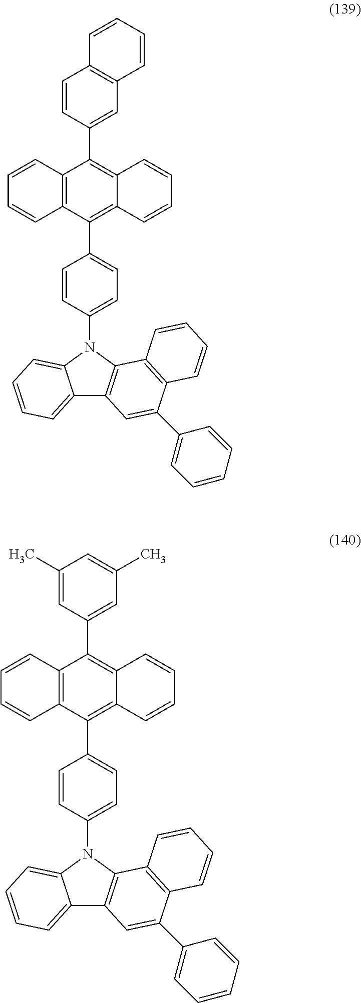 [SMILES: c1ccc(-c2cc3c4ccccc4n(-c4ccc(-c5c6ccccc6c(-c6ccc7ccccc7c6)c6ccccc56)cc4)c3c3ccccc23)cc1 ; Cc1cc(C)cc(-c2c3ccccc3c(-c3ccc(-n4c5ccccc5c5cc(-c6ccccc6)c6ccccc6c54)cc3)c3ccccc23)c1]

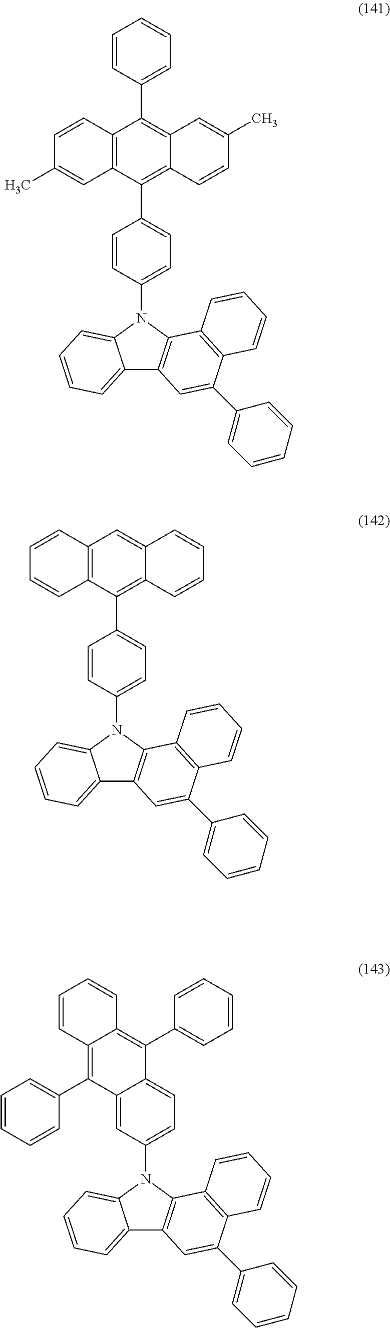 [SMILES: Cc1ccc2c(-c3ccc(-n4c5ccccc5c5cc(-c6ccccc6)c6ccccc6c54)cc3)c3cc(C)ccc3c(-c3ccccc3)c2c1 ; c1ccc(-c2cc3c4ccccc4n(-c4ccc(-c5c6ccccc6cc6ccccc56)cc4)c3c3ccccc23)cc1 ; c1ccc(-c2c3ccccc3c(-c3ccccc3)c3cc(-n4c5ccccc5c5cc(-c6ccccc6)c6ccccc6c54)ccc23)cc1]

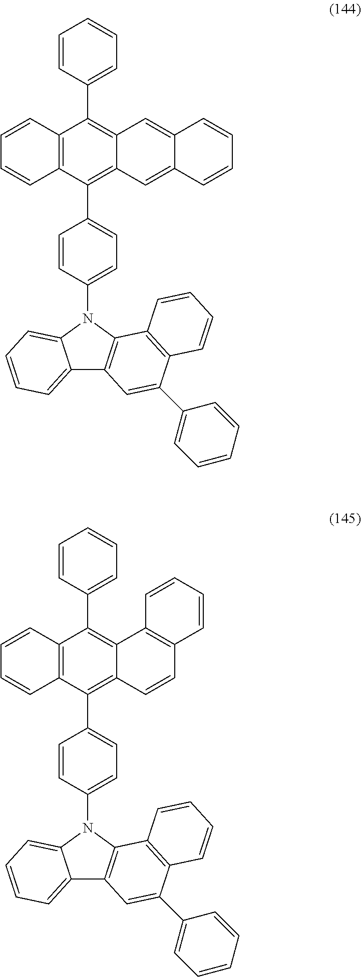 [SMILES: c1ccc(-c2c3ccccc3c(-c3ccc(-n4c5ccccc5c5cc(-c6ccccc6)c6ccccc6c54)cc3)c3cc4ccccc4cc23)cc1 ; c1ccc(-c2cc3c4ccccc4n(-c4ccc(-c5c6ccccc6c(-c6ccccc6)c6c5ccc5ccccc56)cc4)c3c3ccccc23)cc1]

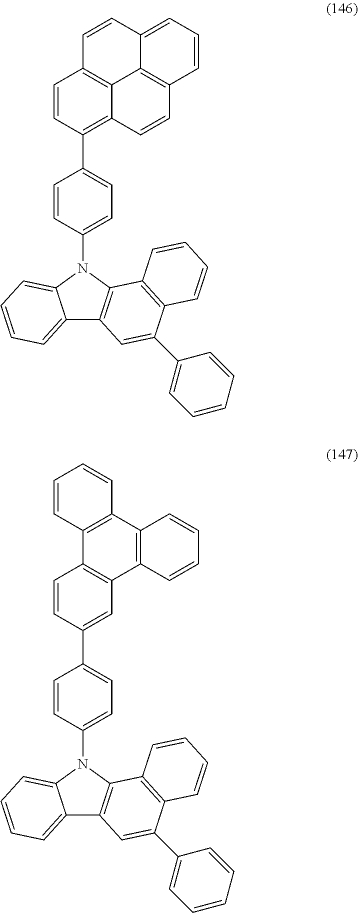 [SMILES: c1ccc(-c2cc3c4ccccc4n(-c4ccc(-c5ccc6ccc7cccc8ccc5c6c78)cc4)c3c3ccccc23)cc1 ; c1ccc(-c2cc3c4ccccc4n(-c4ccc(-c5ccc6c7ccccc7c7ccccc7c6c5)cc4)c3c3ccccc23)cc1]

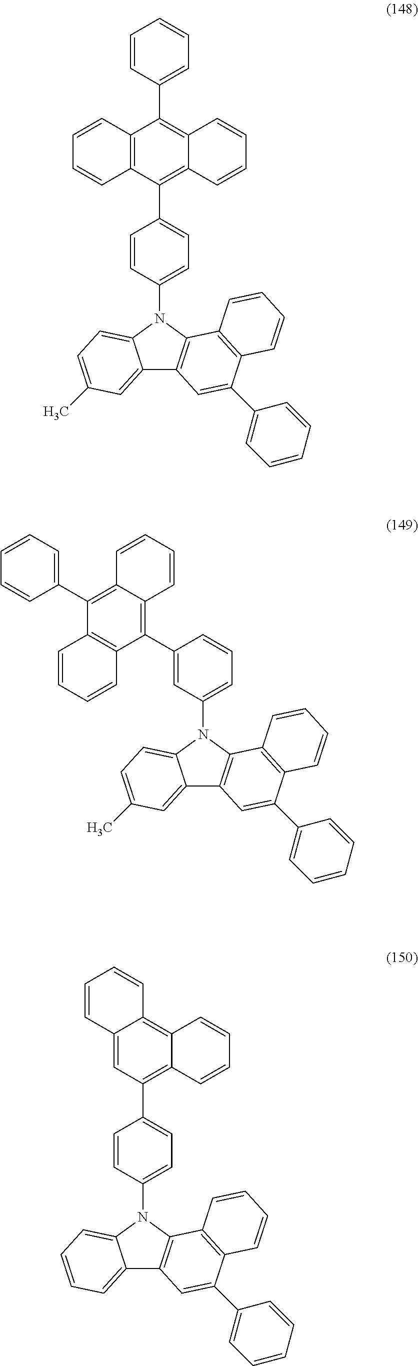 [SMILES: Cc1ccc2c(c1)c1cc(-c3ccccc3)c3ccccc3c1n2-c1ccc(-c2c3ccccc3c(-c3ccccc3)c3ccccc23)cc1 ; Cc1ccc2c(c1)c1cc(-c3ccccc3)c3ccccc3c1n2-c1cccc(-c2c3ccccc3c(-c3ccccc3)c3ccccc23)c1 ; c1ccc(-c2cc3c4ccccc4n(-c4ccc(-c5cc6ccccc6c6ccccc56)cc4)c3c3ccccc23)cc1]

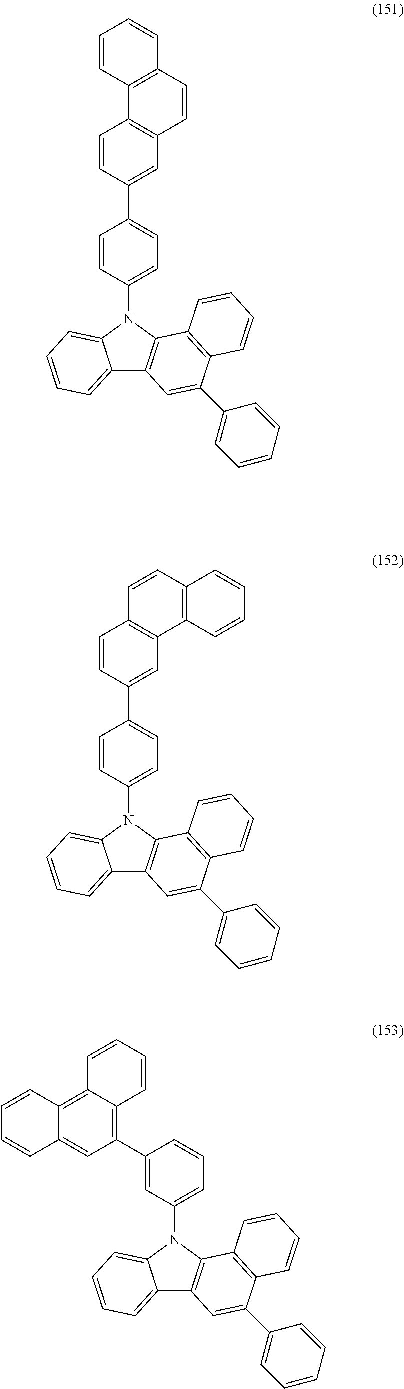 [SMILES: c1ccc(-c2cc3c4ccccc4n(-c4ccc(-c5ccc6c(ccc7ccccc76)c5)cc4)c3c3ccccc23)cc1 ; c1ccc(-c2cc3c4ccccc4n(-c4ccc(-c5ccc6ccc7ccccc7c6c5)cc4)c3c3ccccc23)cc1 ; c1ccc(-c2cc3c4ccccc4n(-c4cccc(-c5cc6ccccc6c6ccccc56)c4)c3c3ccccc23)cc1]

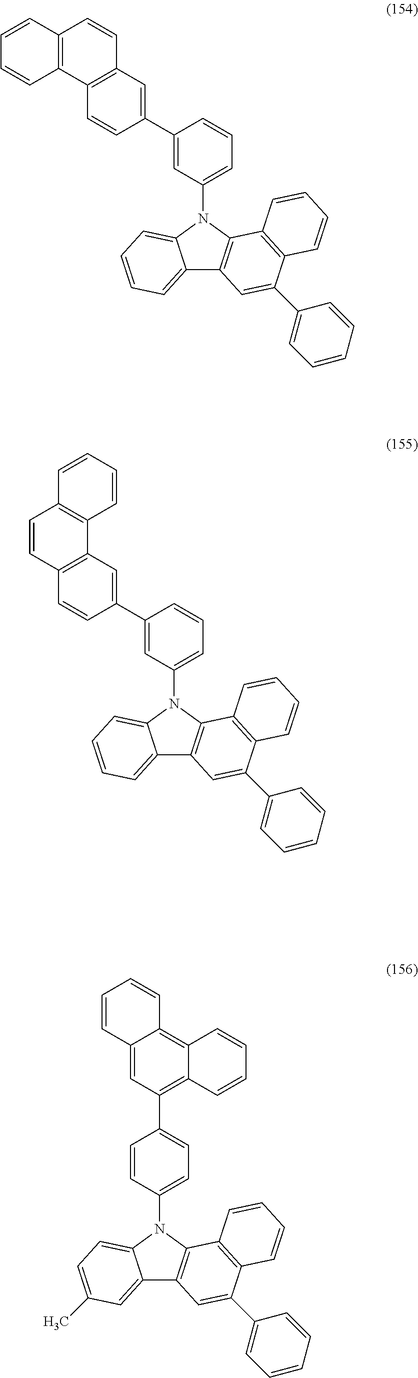 [SMILES: c1ccc(-c2cc3c4ccccc4n(-c4cccc(-c5ccc6c(ccc7ccccc76)c5)c4)c3c3ccccc23)cc1 ; c1ccc(-c2cc3c4ccccc4n(-c4cccc(-c5ccc6ccc7ccccc7c6c5)c4)c3c3ccccc23)cc1 ; Cc1ccc2c(c1)c1cc(-c3ccccc3)c3ccccc3c1n2-c1ccc(-c2cc3ccccc3c3ccccc23)cc1]

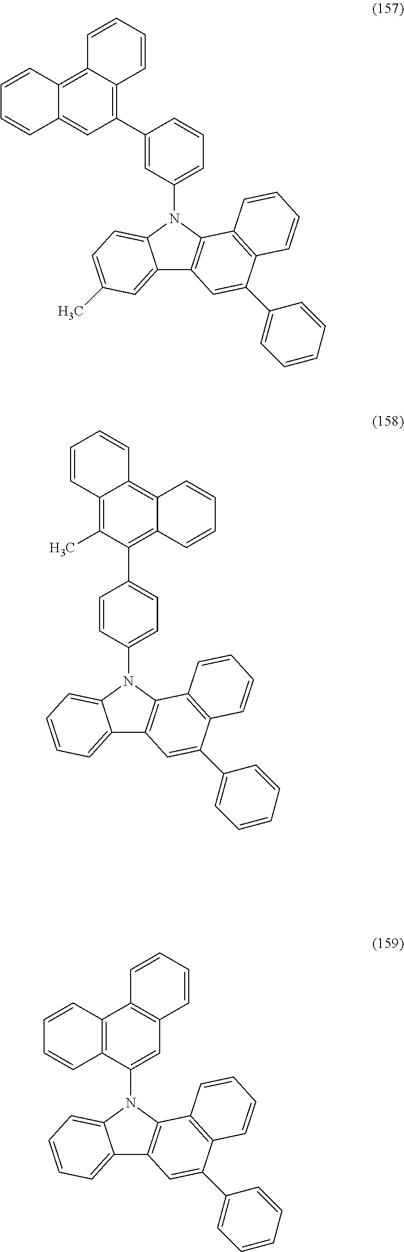 [SMILES: Cc1ccc2c(c1)c1cc(-c3ccccc3)c3ccccc3c1n2-c1cccc(-c2cc3ccccc3c3ccccc23)c1 ; Cc1c(-c2ccc(-n3c4ccccc4c4cc(-c5ccccc5)c5ccccc5c43)cc2)c2ccccc2c2ccccc12 ; c1ccc(-c2cc3c4ccccc4n(-c4cc5ccccc5c5ccccc45)c3c3ccccc23)cc1]

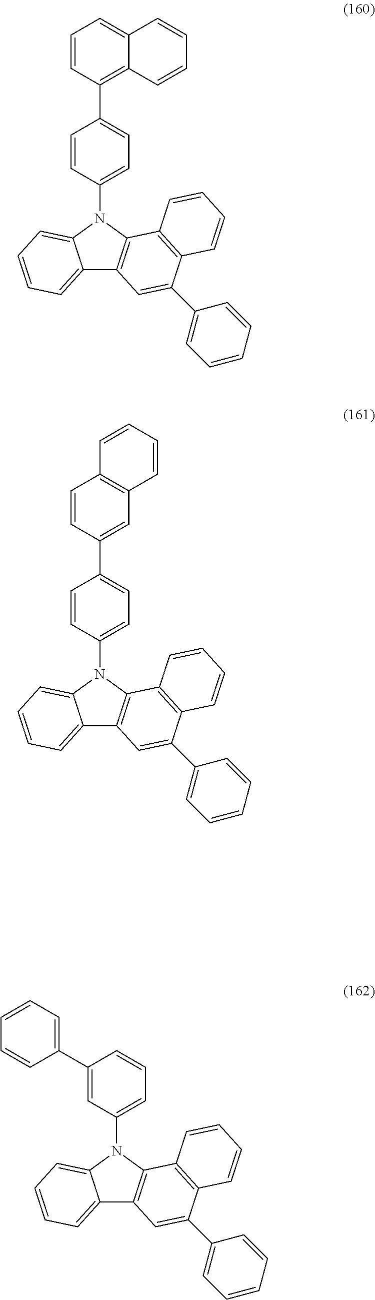 [SMILES: c1ccc(-c2cc3c4ccccc4n(-c4ccc(-c5cccc6ccccc56)cc4)c3c3ccccc23)cc1 ; c1ccc(-c2cc3c4ccccc4n(-c4ccc(-c5ccc6ccccc6c5)cc4)c3c3ccccc23)cc1 ; c1ccc(-c2cccc(-n3c4ccccc4c4cc(-c5ccccc5)c5ccccc5c43)c2)cc1]

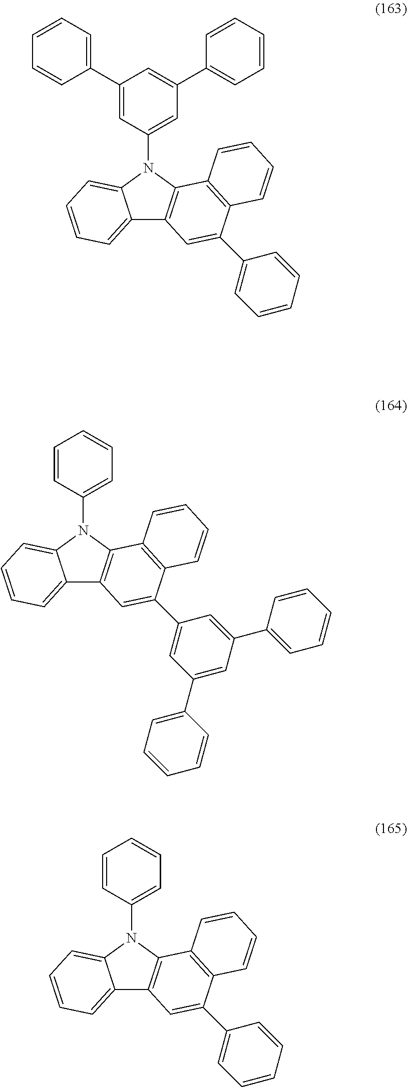 [SMILES: c1ccc(-c2cc(-c3ccccc3)cc(-n3c4ccccc4c4cc(-c5ccccc5)c5ccccc5c43)c2)cc1 ; c1ccc(-c2cc(-c3ccccc3)cc(-c3cc4c5ccccc5n(-c5ccccc5)c4c4ccccc34)c2)cc1 ; c1ccc(-c2cc3c4ccccc4n(-c4ccccc4)c3c3ccccc23)cc1]

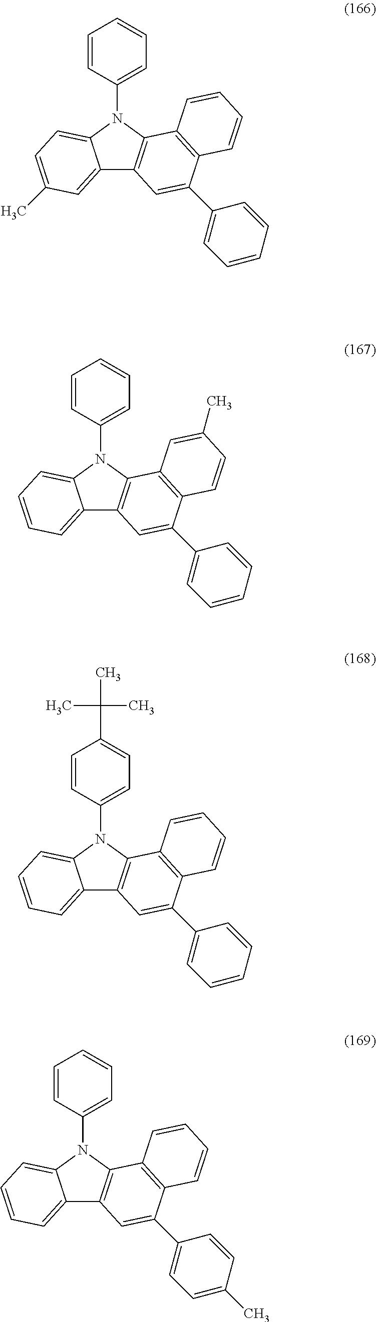 [SMILES: Cc1ccc2c(c1)c1cc(-c3ccccc3)c3ccccc3c1n2-c1ccccc1 ; Cc1ccc2c(-c3ccccc3)cc3c4ccccc4n(-c4ccccc4)c3c2c1 ; CC(C)(C)c1ccc(-n2c3ccccc3c3cc(-c4ccccc4)c4ccccc4c32)cc1 ; Cc1ccc(-c2cc3c4ccccc4n(-c4ccccc4)c3c3ccccc23)cc1]

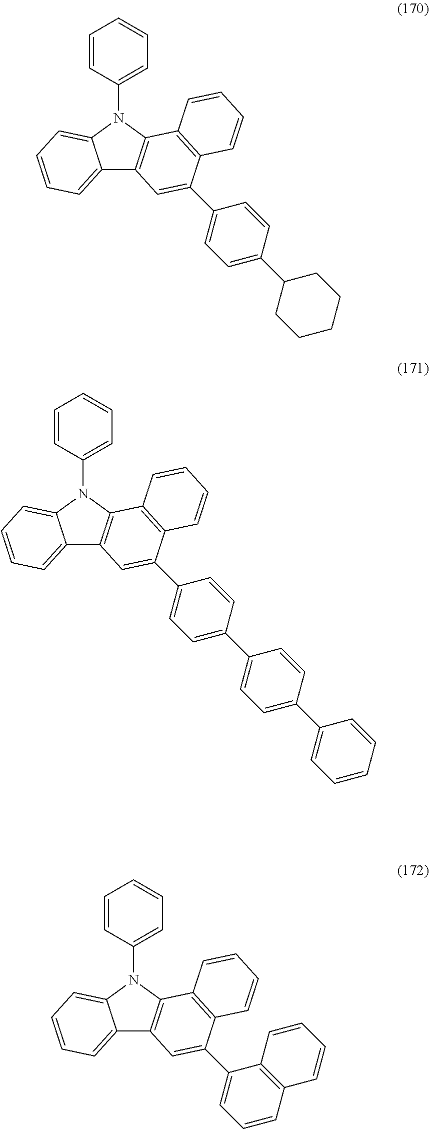 [SMILES: c1ccc(-n2c3ccccc3c3cc(-c4ccc(C5CCCCC5)cc4)c4ccccc4c32)cc1 ; c1ccc(-c2ccc(-c3ccc(-c4cc5c6ccccc6n(-c6ccccc6)c5c5ccccc45)cc3)cc2)cc1 ; c1ccc(-n2c3ccccc3c3cc(-c4cccc5ccccc45)c4ccccc4c32)cc1]

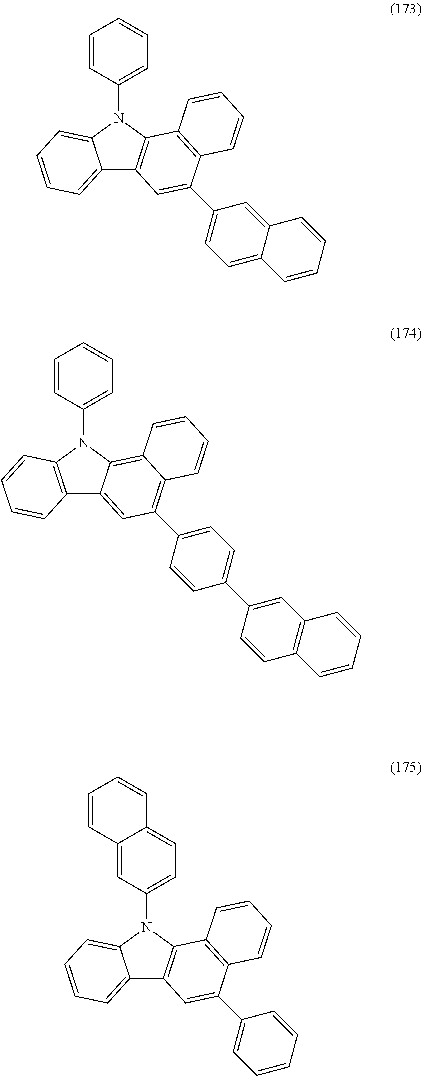 [SMILES: c1ccc(-n2c3ccccc3c3cc(-c4ccc5ccccc5c4)c4ccccc4c32)cc1 ; c1ccc(-n2c3ccccc3c3cc(-c4ccc(-c5ccc6ccccc6c5)cc4)c4ccccc4c32)cc1 ; c1ccc(-c2cc3c4ccccc4n(-c4ccc5ccccc5c4)c3c3ccccc23)cc1]

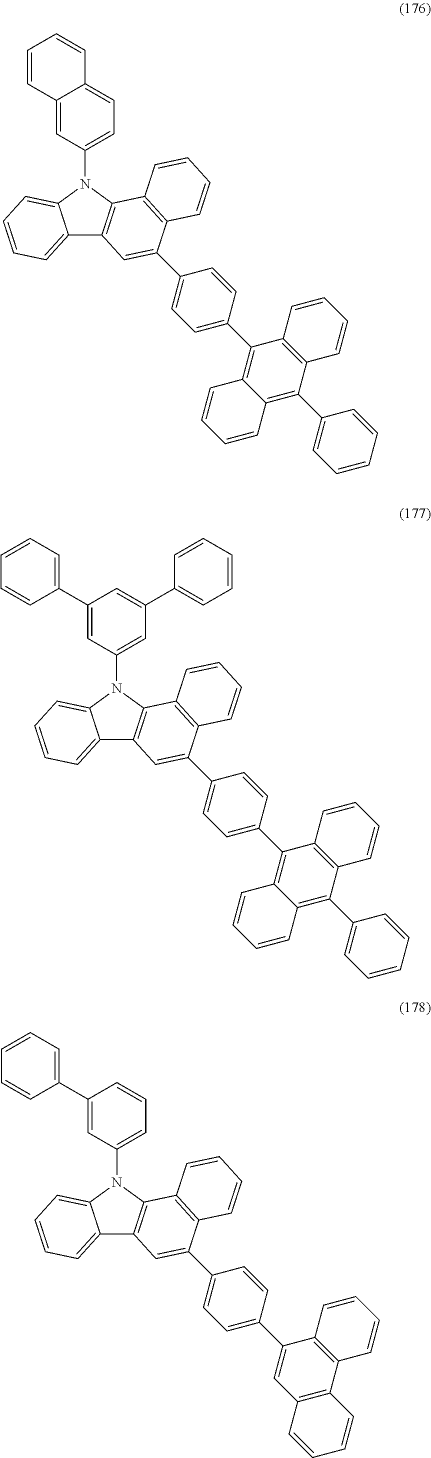 [SMILES: c1ccc(-c2c3ccccc3c(-c3ccc(-c4cc5c6ccccc6n(-c6ccc7ccccc7c6)c5c5ccccc45)cc3)c3ccccc23)cc1 ; c1ccc(-c2cc(-c3ccccc3)cc(-n3c4ccccc4c4cc(-c5ccc(-c6c7ccccc7c(-c7ccccc7)c7ccccc67)cc5)c5ccccc5c43)c2)cc1 ; c1ccc(-c2cccc(-n3c4ccccc4c4cc(-c5ccc(-c6cc7ccccc7c7ccccc67)cc5)c5ccccc5c43)c2)cc1]

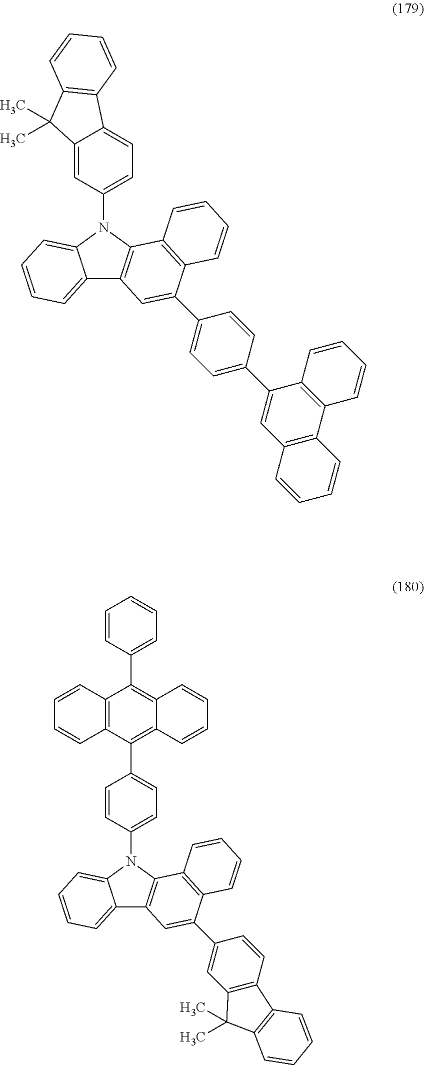 [SMILES: CC1(C)c2ccccc2-c2ccc(-n3c4ccccc4c4cc(-c5ccc(-c6cc7ccccc7c7ccccc67)cc5)c5ccccc5c43)cc21 ; CC1(C)c2ccccc2-c2ccc(-c3cc4c5ccccc5n(-c5ccc(-c6c7ccccc7c(-c7ccccc7)c7ccccc67)cc5)c4c4ccccc34)cc21]

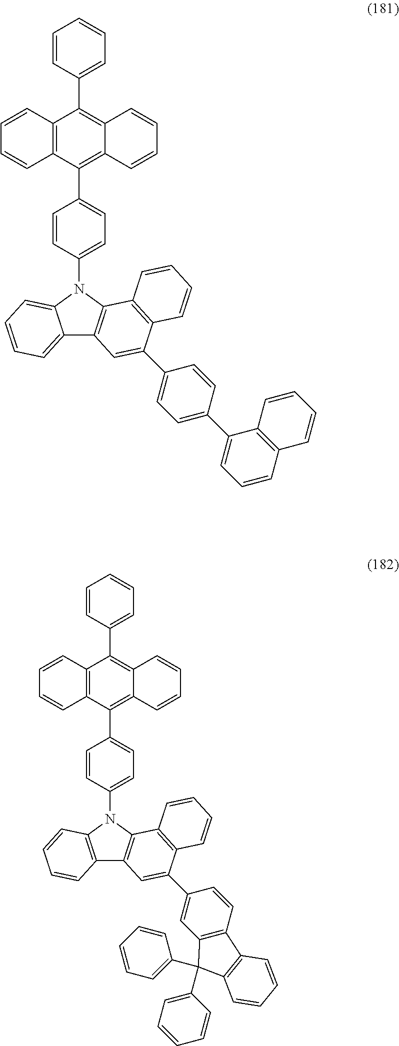 [SMILES: c1ccc(-c2c3ccccc3c(-c3ccc(-n4c5ccccc5c5cc(-c6ccc(-c7cccc8ccccc78)cc6)c6ccccc6c54)cc3)c3ccccc23)cc1 ; c1ccc(-c2c3ccccc3c(-c3ccc(-n4c5ccccc5c5cc(-c6ccc7c(c6)C(c6ccccc6)(c6ccccc6)c6ccccc6-7)c6ccccc6c54)cc3)c3ccccc23)cc1]

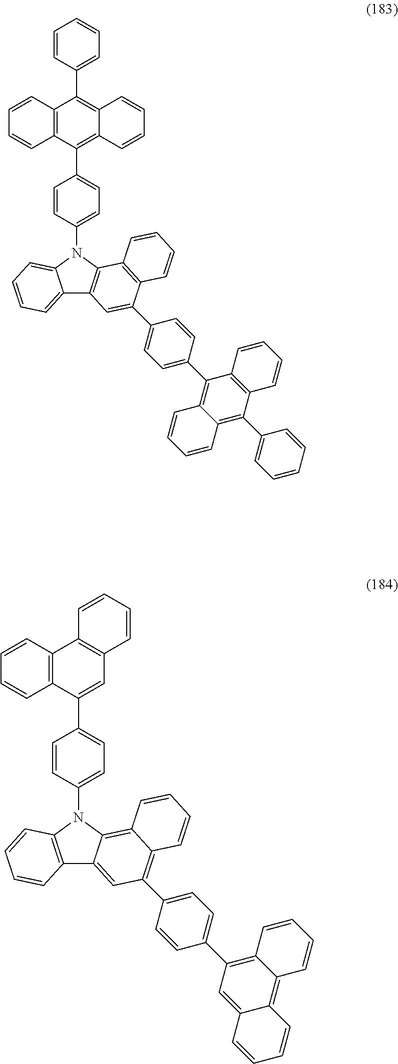 [SMILES: c1ccc(-c2c3ccccc3c(-c3ccc(-c4cc5c6ccccc6n(-c6ccc(-c7c8ccccc8c(-c8ccccc8)c8ccccc78)cc6)c5c5ccccc45)cc3)c3ccccc23)cc1 ; c1ccc2c(c1)cc(-c1ccc(-c3cc4c5ccccc5n(-c5ccc(-c6cc7ccccc7c7ccccc67)cc5)c4c4ccccc34)cc1)c1ccccc12]

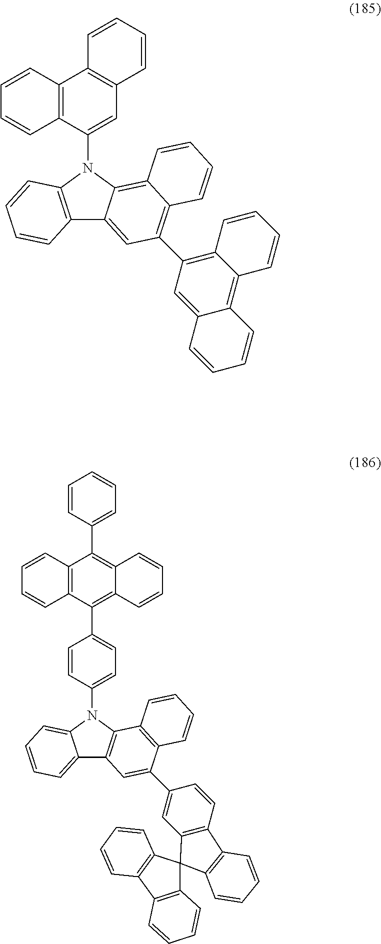 [SMILES: c1ccc2c(c1)cc(-c1cc3c4ccccc4n(-c4cc5ccccc5c5ccccc45)c3c3ccccc13)c1ccccc12 ; c1ccc(-c2c3ccccc3c(-c3ccc(-n4c5ccccc5c5cc(-c6ccc7c(c6)C6(c8ccccc8-c8ccccc86)c6ccccc6-7)c6ccccc6c54)cc3)c3ccccc23)cc1]

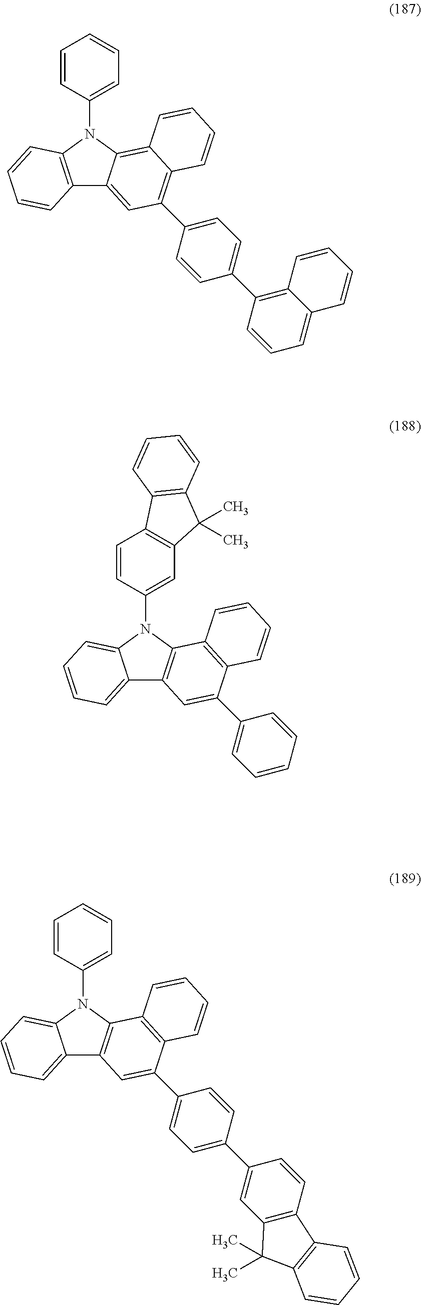 [SMILES: c1ccc(-n2c3ccccc3c3cc(-c4ccc(-c5cccc6ccccc56)cc4)c4ccccc4c32)cc1 ; CC1(C)c2ccccc2-c2ccc(-n3c4ccccc4c4cc(-c5ccccc5)c5ccccc5c43)cc21 ; CC1(C)c2ccccc2-c2ccc(-c3ccc(-c4cc5c6ccccc6n(-c6ccccc6)c5c5ccccc45)cc3)cc21]

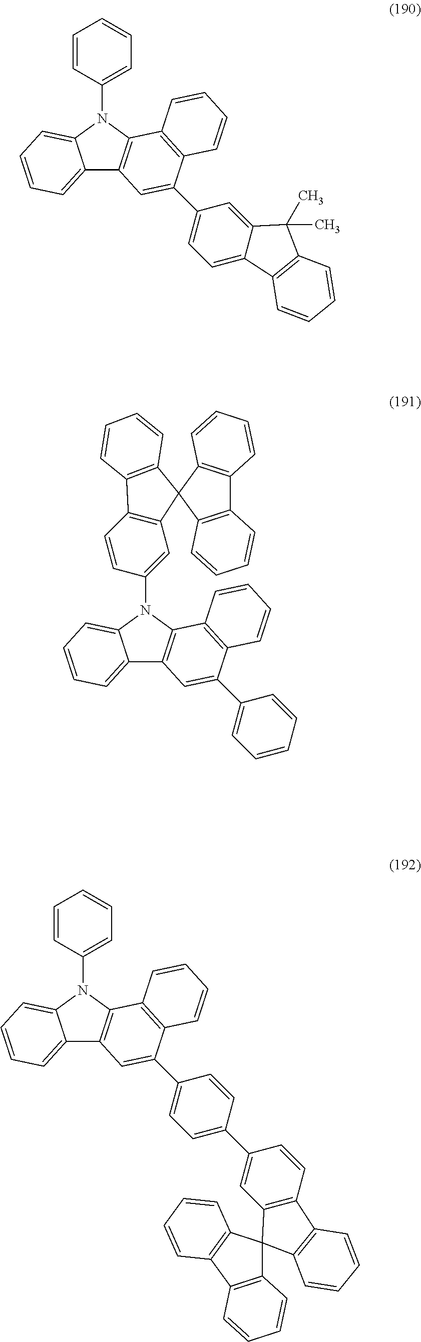 [SMILES: CC1(C)c2ccccc2-c2ccc(-c3cc4c5ccccc5n(-c5ccccc5)c4c4ccccc34)cc21 ; c1ccc(-c2cc3c4ccccc4n(-c4ccc5c(c4)C4(c6ccccc6-c6ccccc64)c4ccccc4-5)c3c3ccccc23)cc1 ; c1ccc(-n2c3ccccc3c3cc(-c4ccc(-c5ccc6c(c5)C5(c7ccccc7-c7ccccc75)c5ccccc5-6)cc4)c4ccccc4c32)cc1]

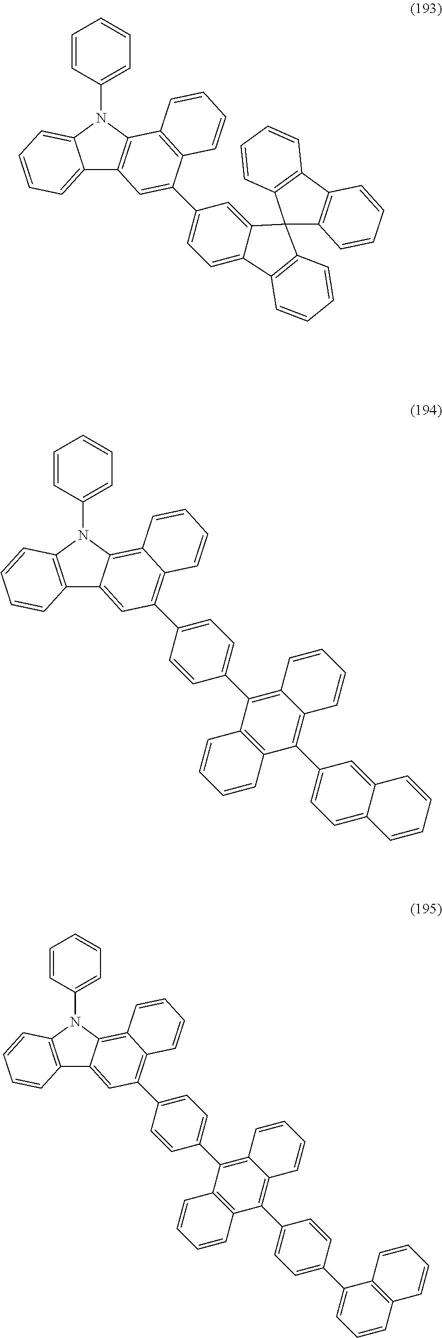 [SMILES: c1ccc(-n2c3ccccc3c3cc(-c4ccc5c(c4)C4(c6ccccc6-c6ccccc64)c4ccccc4-5)c4ccccc4c32)cc1 ; c1ccc(-n2c3ccccc3c3cc(-c4ccc(-c5c6ccccc6c(-c6ccc7ccccc7c6)c6ccccc56)cc4)c4ccccc4c32)cc1 ; c1ccc(-n2c3ccccc3c3cc(-c4ccc(-c5c6ccccc6c(-c6ccc(-c7cccc8ccccc78)cc6)c6ccccc56)cc4)c4ccccc4c32)cc1]

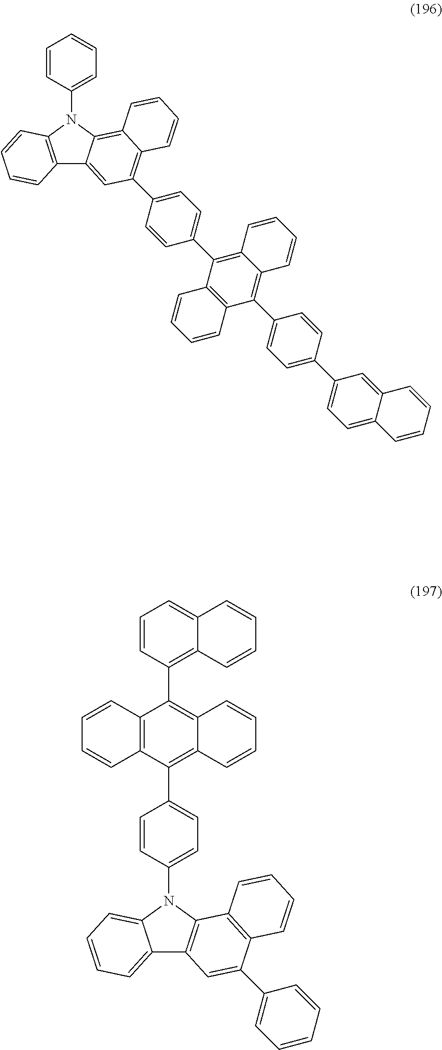 [SMILES: c1ccc(-n2c3ccccc3c3cc(-c4ccc(-c5c6ccccc6c(-c6ccc(-c7ccc8ccccc8c7)cc6)c6ccccc56)cc4)c4ccccc4c32)cc1 ; c1ccc(-c2cc3c4ccccc4n(-c4ccc(-c5c6ccccc6c(-c6cccc7ccccc67)c6ccccc56)cc4)c3c3ccccc23)cc1]

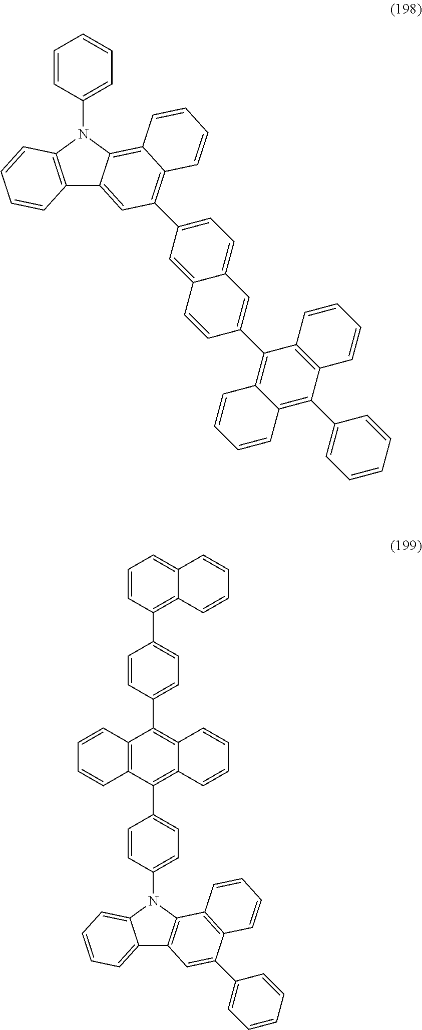 [SMILES: c1ccc(-c2c3ccccc3c(-c3ccc4cc(-c5cc6c7ccccc7n(-c7ccccc7)c6c6ccccc56)ccc4c3)c3ccccc23)cc1 ; c1ccc(-c2cc3c4ccccc4n(-c4ccc(-c5c6ccccc6c(-c6ccc(-c7cccc8ccccc78)cc6)c6ccccc56)cc4)c3c3ccccc23)cc1]

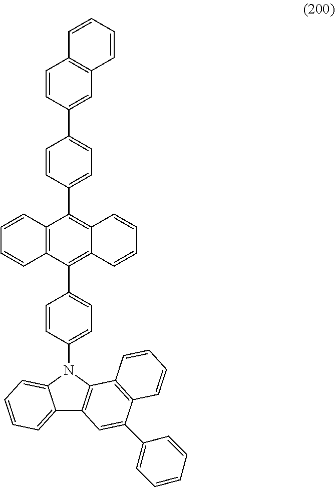 [SMILES: c1ccc(-c2cc3c4ccccc4n(-c4ccc(-c5c6ccccc6c(-c6ccc(-c7ccc8ccccc8c7)cc6)c6ccccc56)cc4)c3c3ccccc23)cc1]

A method for synthesizing a benzo[a]carbazole compound that is an organic compound of one embodiment of the present invention described above and represented by General Formula (G1) shown below is described.

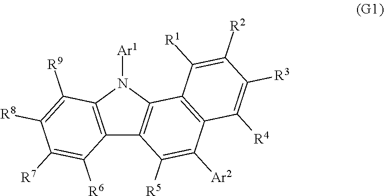

A variety of reactions can be applied to the method of synthesizing the benzo[a]carbazole compound. For example, synthesis reactions described below enable the synthesis of the benzo[a]carbazole compound represented by General Formula (G1). Note that the method of synthesizing the benzo[a]carbazole compound of one embodiment of the present invention is not limited to the following synthesis methods.

### Synthesis Method 1 of Benzo[a]Carbazole Compound Represented by General Formula (G1)

A benzo[a]carbazole compound (a3) can be synthesized as in Synthesis Scheme (A-1) shown below. That is, a benzo[a]carbazole derivative (a1) and a halide of an aryl derivative (a2) are coupled using a metal catalyst, a metal, or a metal compound in the presence of a base, whereby the benzo[a]carbazole compound (a3) is obtained. Synthesis Scheme (A-1) of this reaction is shown below.

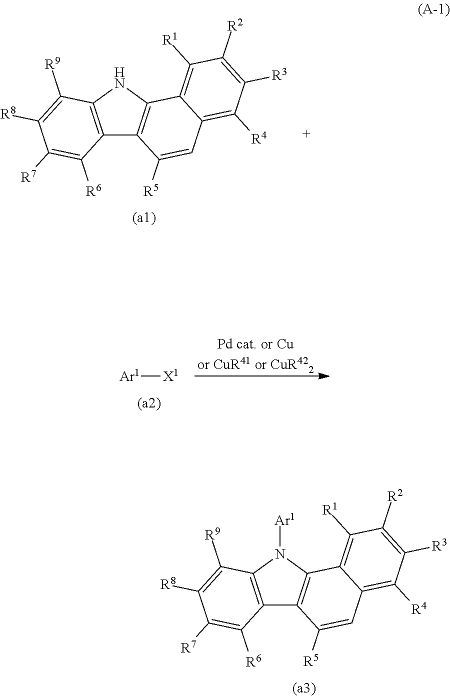

In Synthesis Scheme (A-1), Ar¹ represents a substituted or unsubstituted aromatic hydrocarbon group having 6 to 30 carbon atoms; each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and each of R⁶ to R⁹ independently represents any of hydrogen, a saturated hydrocarbon group having 1 to 6 carbon atoms, and a cyclic saturated hydrocarbon group having 3 to 6 carbon atoms.

In the case where a Hartwig-Buchwald reaction is performed in Synthesis Scheme (A-1), X¹ represents a halogen or a triflate group. As the halogen, iodine, bromine, or chlorine is preferable. In this reaction, a palladium catalyst including a palladium compound or a palladium complex such as bis(dibenzylideneacetone)palladium(0) or palladium(II) acetate and a ligand that coordinates to the palladium complex or the palladium compound, such as tri(tert-butyl)phosphine, tri(n-hexyl)phosphine, or tricyclohexylphosphine, is used. Examples of the base include organic bases such as sodium tert-butoxide and inorganic bases such as a potassium carbonate. In the case where a solvent is used, toluene, xylene, benzene, tetrahydrofuran, or the like can be used.

In the case where an Ullmann reaction is performed in Synthesis Scheme (A-1), X¹ represents a halogen. As the halogen, iodine, bromine, or chlorine is preferable. As a catalyst, copper or a copper compound is used. In the case where a copper compound is used as the catalyst, each of R⁴¹ and R⁴² in Synthesis Scheme (A-1) individually represents a halogen, an acetyl group, or the like. As the halogen, chlorine, bromine, or iodine can be used. Note that copper(I) iodide where R⁴¹ is iodine or copper(II) acetate where R⁴² is an acetyl group is preferably used. As the base, an inorganic base such as potassium carbonate can be used. As a solvent, 1,3-dimethyl-3,4,5,6-tetrahydro-2(1H)pyrimidinone (DMPU), toluene, xylene, benzene, or the like can be employed. However, the solvent is not limited thereto. In the Ullmann reaction, when the reaction temperature is 100° C. or higher, a target substance can be obtained in a shorter time in a higher yield; therefore, it is preferable to use DMPU or xylene each having a high boiling point. In addition, since the reaction temperature is more preferably 150° C. or higher, DMPU is more preferably used.

Next, a halogenated benzo[a]carbazole compound (a4) can be synthesized as in Synthesis Scheme (A-2) shown below. That is, the benzo[a]carbazole compound (a3) is halogenated with a halogenating agent, whereby the halogenated benzo[a]carbazole compound (a4) can be obtained. Synthesis Scheme (A-2) of this reaction is shown below.

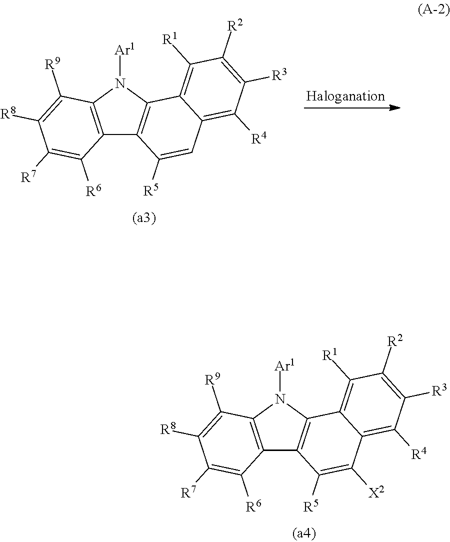

In Synthesis Scheme (A-2), X² represents a halogen. The halogen is preferably iodine or bromine.

In the case where bromination is performed in Synthesis Scheme (A-2), examples of a brominating agent which can be used include bromine and N-bromosuccinimide. Examples of a solvent which can be used in the case of bromination using bromine include a halogen-based solvent such as chloroform or carbon tetrachloride. Examples of a solvent which can be used in the case of bromination using N-bromosuccinimide include ethyl acetate, tetrahydrofuran, dimethylformamide, acetic acid, and water.

In the case where iodination is performed in Synthesis Scheme (A-2), examples of an iodinating agent which can be used include N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodination using any of those iodinating agents include aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogens such as dichloromethane, chloroform, carbon tetrachloride, 1,2-dichloroethane, and 1,1,1-trichloroethane; nitriles such as acetonitrile and benzonitrile; esters such as ethyl acetate, methyl acetate, and butyl acetate; acetic acid (glacial acetic acid); and water. Those solvents can be used alone or in combination. When water is used, it is preferably mixed with an organic solvent. In addition, in this reaction, an acid such as sulfuric acid or acetic acid is preferably used as well.

Next, a boronic acid or an organoboron compound of a benzo[a]carbazole compound (a5) can be synthesized as in Synthesis Scheme (A-3). That is, the halogenated benzo[a]carbazole compound (a4) is converted into a boronic acid or an organoboron compound with the use of an alkyllithium reagent and a boron reagent, whereby the boronic acid or organoboron compound of the organoboron compound (a5) is obtained.

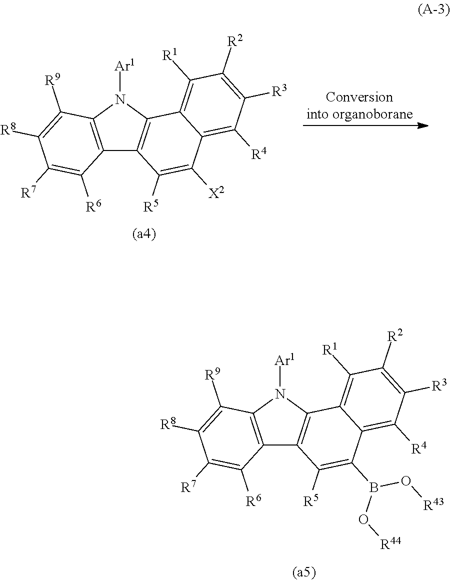

In Synthesis Scheme (A-3), when the compound (a5) is a boronic acid, each of R⁴³ and R⁴⁴ represents hydrogen. In addition, the boronic acid of the compound (a5) may be protected by ethylene glycol or the like, and in this case, each of R⁴³ and R⁴⁴ in the compound (a5) represents an alkyl group having 1 to 6 carbon atoms. In the case where the compound (a5) is an organoboron compound, R⁴³ and R⁴⁴ may be the same or different and bonded to each other to form a ring.

In the reaction in Synthesis Scheme (A-3), an ether-based solvent such as diethyl ether, tetrahydrofuran (THF), or cyclopentyl methyl ether can be used. However, the solvent that can be used is not limited thereto. The alkyllithium reagent may be, but not limited to, n-butyllithium, sec-butyl lithium, tert-butyl lithium, or the like. Furthermore, addition of a coordinating additive to such an alkyllithium reagent can enhance reactivity. The coordinating additive may be, but not limited to, tetramethylethylenediamine (TMEDA) or the like. In addition, the boron reagent may be, but not limited to, trimethyl borate, triisopropyl borate, or the like.

Next, a boronic acid of the benzo[a]carbazole compound or an organoboron compound of the benzo[a]carbazole compound (a5) and a halide of an aryl derivative (a6) are coupled using a metal catalyst, a metal, or a metal compound in the presence of a base, whereby the benzo[a]carbazole compound (G1) that is an organic compound of one embodiment of the present invention can be obtained. Synthesis Scheme (A-4) of this reaction is shown below.

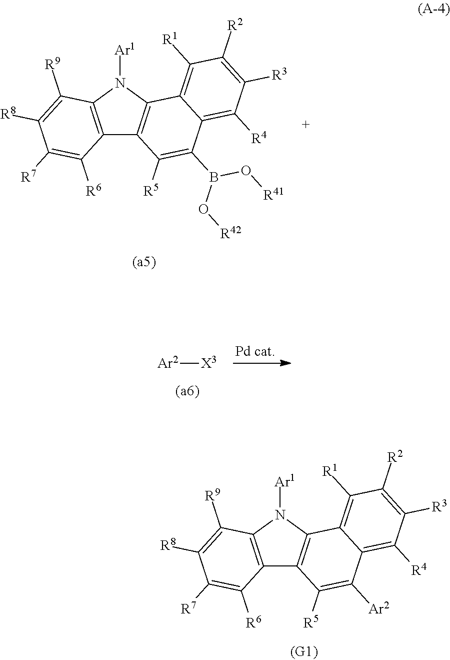

In Synthesis Scheme (A-4), X³ represents a halogen. As the halogen, iodine, bromine, or chlorine is preferable. Ar² represents a substituted or unsubstituted aromatic hydrocarbon group having 6 to 100 carbon atoms.

In the reaction in Synthesis Scheme (A-4), a palladium catalyst including a palladium compound or a palladium complex such as palladium(II) acetate or tetrakis(triphenylphosphine)palladium(0) and a ligand that coordinates to the palladium complex or the palladium compound, such as tri(ortho-tolyl)phosphine or tricyclohexylphosphine, is used. As the base, an organic base such as sodium tert-butoxide, an inorganic base such as potassium carbonate or sodium carbonate, and the like can be used. Examples of solvents are as follows: a mixed solvent of toluene and water; a mixed solvent of toluene, an alcohol such as ethanol, and water; a mixed solvent of xylene and water; a mixed solvent of xylene, an alcohol such as ethanol, and water; a mixed solvent of benzene and water; a mixed solvent of benzene, an alcohol such as ethanol, and water; and a mixed solvent of an ether such as 1,2-dimethoxyethane, and water. In particular, a mixed solvent of toluene and water or a mixed solvent of toluene, ethanol, and water is preferable.

Although the boronic acid of the benzo[a]carbazole compound or the organoboron compound of the benzo[a]carbazole compound (a5) and the halide of an aryl derivative (a6) are reacted in Synthesis Scheme (A-4) shown above, the benzo[a]carbazole compound (G1) can be synthesized by causing a reaction between the compounds (a5) and (a6) whose reactive groups (the boron compound group and the halogen group) are switched.

The benzo[a]carbazole compound (G1) of one embodiment of the present invention can also be synthesized through the synthesis reaction described below.

### Synthesis Method 2 of Benzo[a]Carbazole Compound Represented by General Formula (G1)

First, a halogenated benzo[a]carbazole compound (b2) can be synthesized as in Synthesis Scheme (B-1) shown below. That is, the benzo[a]carbazole compound (b1) is halogenated with a halogenating agent, whereby the halogenated benzo[a]carbazole compound (b2) can be obtained. Synthesis Scheme (B-1) of this reaction is shown below.

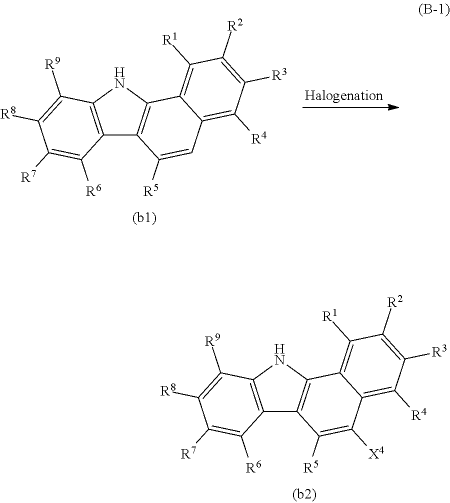

In Synthesis Scheme (B-1), X⁴ represents a halogen. As the halogen, iodine or bromine is preferable.

In the case where bromination is performed in Synthesis Scheme (B-1), examples of a brominating agent which can be used include bromine and N-bromosuccinimide. Examples of a solvent which can be used in the case of bromination using bromine include a halogen-based solvent such as chloroform or carbon tetrachloride. Examples of a solvent which can be used in the case of bromination using N-bromosuccinimide include ethyl acetate, tetrahydrofuran, dimethylformamide, acetic acid, and water.

In the case where iodination is performed in Synthesis Scheme (B-1), examples of an iodinating agent which can be used include N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodination using any of those iodinating agents include aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogens such as dichloromethane, chloroform, carbon tetrachloride, 1,2-dichloroethane, and 1,1,1-trichloroethane; nitriles such as acetonitrile and benzonitrile; esters such as ethyl acetate, methyl acetate, and butyl acetate; acetic acid (glacial acetic acid); and water. Those solvents can be used alone or in combination. When water is used, it is preferably mixed with an organic solvent. In addition, in this reaction, an acid such as sulfuric acid or acetic acid is preferably used as well.

Next, a benzo[a]carbazole compound (b4) can be synthesized as in Synthesis Scheme (B-2) shown above. That is, the halogenated benzo[a]carbazole compound (b2) and a boronic acid of an aryl compound or an organoboron compound of an aryl compound (b3) are coupled using a metal catalyst, a metal, or a metal compound in the presence of a base, whereby the benzo[a]carbazole compound (b4) can be obtained. Synthesis Scheme (B-2) of this reaction is shown below.

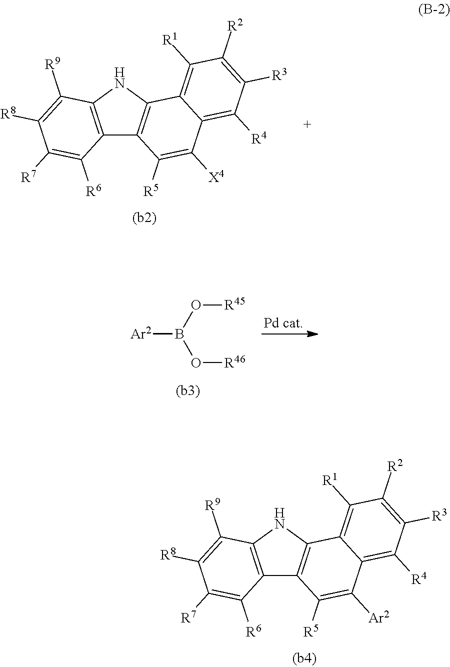

In the reaction in Synthesis Scheme (B-2), a palladium catalyst including a palladium compound or a palladium complex such as palladium(II) acetate or tetrakis(triphenylphosphine)palladium(0) and a ligand that coordinates to the palladium complex or the palladium compound, such as tri(ortho-tolyl)phosphine or tricyclohexylphosphine, is used. As the base, an organic base such as sodium tert-butoxide, an inorganic base such as potassium carbonate or sodium carbonate, and the like can be used. Examples of solvents are as follows: a mixed solvent of toluene and water; a mixed solvent of toluene, an alcohol such as ethanol, and water; a mixed solvent of xylene and water; a mixed solvent of xylene, an alcohol such as ethanol, and water; a mixed solvent of benzene and water; a mixed solvent of benzene, an alcohol such as ethanol, and water; and a mixed solvent of an ether such as 1,2-dimethoxyethane, and water. In particular, a mixed solvent of toluene and water or a mixed solvent of toluene, ethanol, and water is preferable.

Although the halogenated benzo[a]carbazole compound (b2) and the boronic acid or organoboron compound of the aryl compound (b3) are reacted in Synthesis Scheme (B-2) shown above, the benzo[a]carbazole compound (G1) can be synthesized by causing a reaction between the compounds (b2) and (b3) whose reactive groups (the boron compound group and the halogen group) are switched.

Finally, the benzo[a]carbazole compound (b4) and a halogenated aryl compound (b5) are coupled using a metal catalyst, a metal, or a metal compound in the presence of a base, whereby the benzo[a]carbazole compound (G1) of one embodiment of the present invention can be obtained. Synthesis Scheme (B-3) of this reaction is shown below.

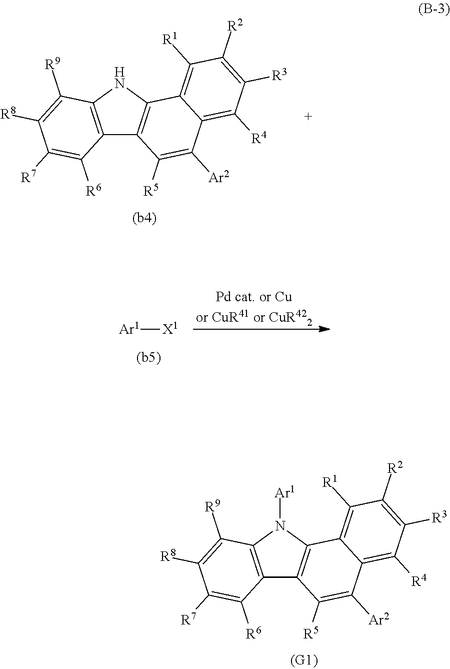

In the case where a Hartwig-Buchwald reaction is performed in Synthesis Scheme (B-3), X¹ represents a halogen or a triflate group. As the halogen, iodine, bromine, or chlorine is preferable. In this reaction, a palladium catalyst including a palladium compound or a palladium complex such as bis(dibenzylideneacetone)palladium(0) or palladium(II) acetate and a ligand that coordinates to the palladium complex or the palladium compound, such as tri(tert-butyl)phosphine, tri(n-hexyl)phosphine, or tricyclohexylphosphine, is used. Examples of the base include organic bases such as sodium tert-butoxide and inorganic bases such as a potassium carbonate. In the case where a solvent is used, toluene, xylene, benzene, tetrahydrofuran, or the like can be used.

In the case where an Ulhmann reaction is performed in Synthesis Scheme (B-3), X¹ represents a halogen. As the halogen, iodine, bromine, or chlorine is preferable. As a catalyst, copper or a copper compound is used. In the case where a copper compound is used as the catalyst, each of R⁴¹ and R⁴² in Synthesis Scheme (B-3) individually represents a halogen, an acetyl group, or the like. As the halogen, chlorine, bromine, or iodine can be used. Note that copper(I) iodide where R⁴¹ is iodine or copper(II) acetate where R⁴² is an acetyl group is preferably used. As the base, an inorganic base such as potassium carbonate can be used. As a solvent, 1,3-dimethyl-3,4,5,6-tetrahydro-2(1H)pyrimidinone (DMPU), toluene, xylene, benzene, or the like can be employed. However, the solvent is not limited thereto. In the Ullmann reaction, when the reaction temperature is 100° C. or higher, a target substance can be obtained in a shorter time in a higher yield; therefore, it is preferable to use DMPU or xylene each having a high boiling point. In addition, since the reaction temperature is more preferably 150° C. or higher, DMPU is more preferably used.

The benzo[a]carbazole compound (G1) that is an organic compound of one embodiment of the present invention can be synthesized as described above.

The benzo[a]carbazole compounds represented by General and Structural Formulae shown above can be suitably used as a light-emitting element material, and one embodiment of the present invention is a light-emitting element material that includes any of the benzo[a]carbazole compounds represented by General and Structural Formulae shown above. The light-emitting element material of one embodiment of the present invention has a high carrier-transport property, and thus can be suitably used in a carrier-transport layer of a light-emitting element and as a host material in a light-emitting layer of the light-emitting element.

The light-emitting element material of one embodiment of the present invention has a high hole-transport property and thus can be suitably used as a material of a hole-transport layer in a light-emitting element. The light-emitting element material of one embodiment of the present invention which includes the benzo[a]carbazole compound including a fused polycyclic aromatic hydrocarbon skeleton also has a high electron-transport property, and thus can be suitably used in an electron-transport layer or as a host material in a light-emitting layer in a light-emitting element.

When the light-emitting element material of one embodiment of the present invention is used as a material of a carrier-transport layer particularly in a light-emitting element in which a material including an anthracene skeleton is used as a host material in a light-emitting layer, a carrier is easily injected to the light-emitting layer, bringing about preferable effects such as an improvement in reliability of the light-emitting element and suppression of an increase in driving voltage. In particular, the light-emitting element material can be suitably used as a material of the hole-transport layer in contact with the light-emitting layer including the host material including an anthracene skeleton. It is more preferable that the host material including an anthracene skeleton further include a carbazole skeleton.

### <<Light-Emitting Element>>

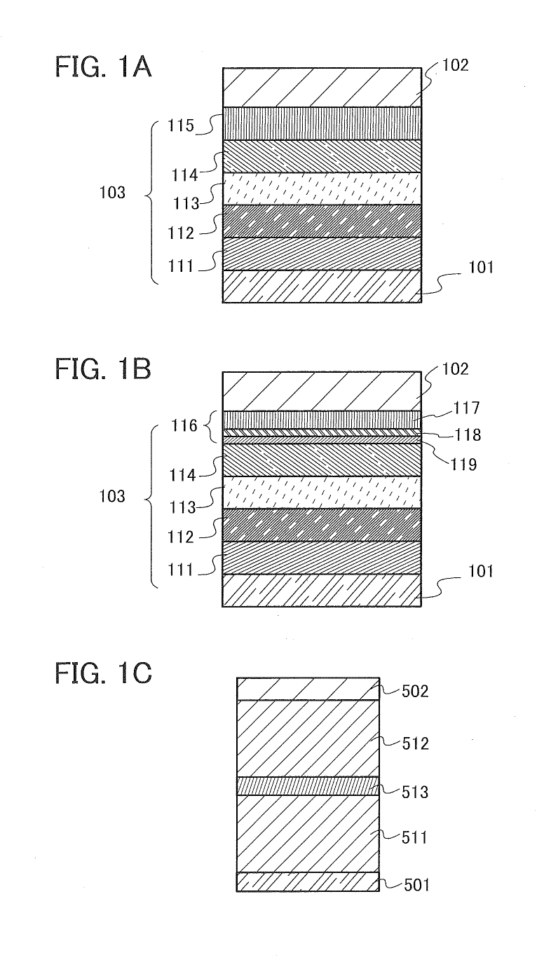

Next, an example of a light-emitting element of one embodiment of the present invention is described in detail below with reference to FIG. 1A.

In this embodiment, the light-emitting element includes a pair of electrodes (a first electrode 101 and a second electrode 102), and an EL layer 103 provided between the first electrode 101 and the second electrode 102. The following description is made on the assumption that the first electrode 101 functions as an anode and the second electrode 102 functions as a cathode.

To function as an anode, the first electrode 101 is preferably formed using any of metals, alloys, conductive compounds having a high work function (specifically, a work function of 4.0 eV or more), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Films of such conductive metal oxides are usually formed by a sputtering method, but may be formed by application of a sol-gel method or the like. In an example of the formation method, indium oxide-zinc oxide is deposited by a sputtering method using a target obtained by adding 1 wt % to 20 wt % of zinc oxide to indium oxide. Furthermore, indium oxide containing tungsten oxide and zinc oxide (IWZO) can be formed by a sputtering method using a target in which tungsten oxide and zinc oxide are added to indium oxide at 0.5 wt % to 5 wt % and 0.1 wt % to 1 wt %, respectively. Other examples are gold (Au), platinum (Pt), nickel (Ni), tungsten (W), chromium (Cr), molybdenum (Mo), iron (Fe), cobalt (Co), copper (Cu), palladium (Pd), nitrides of metal materials (e.g., titanium nitride), and the like. Graphene can also be used. Note that when a composite material described later is used for a layer which is in contact with the first electrode 101 in the EL layer 103, an electrode material can be selected regardless of its work function.

It is preferable that the EL layer 103 have a stacked-layer structure and any of the layers of the stacked-layer structure contain the organic compound represented by any one of General Formulae (G1) to (G6) shown above.

The stacked-layer structure of the EL layer 103 can be formed by combining a hole-injection layer, a hole-transport layer, a light-emitting layer, an electron-transport layer, an electron-injection layer, a carrier-blocking layer, an intermediate layer, and the like as appropriate. In this embodiment, the EL layer 103 has a structure in which a hole-injection layer 111, a hole-transport layer 112, a light-emitting layer 113, an electron-transport layer 114, and an electron-injection layer 115 are stacked in this order over the first electrode 101. Specific examples of the materials forming the layers are given below.

The hole-injection layer 111 is a layer that contains a substance with a high hole-injection property. Molybdenum oxide, vanadium oxide, ruthenium oxide, tungsten oxide, manganese oxide, or the like can be used. Alternatively, the hole-injection layer 111 can be formed using a phthalocyanine-based compound such as phthalocyanine (abbreviation: H₂Pc) or copper phthalocyanine (abbreviation: CuPc), an aromatic amine compound such as 4,4′-bis[N-(4-diphenylaminophenyl)-N-phenylamino]biphenyl (abbreviation: DPAB) or N,N′-bis{4-[bis(3-methylphenyl)amino]phenyl}-N,N′-diphenyl-(1,1′-biphenyl)-4,4′-diamine (abbreviation: DNTPD), a high molecular compound such as poly(3,4-ethylenedioxythiophene)/poly(styrenesulfonic acid) (PEDOT/PSS), or the like.

Alternatively, a composite material in which a substance having a hole-transport property contains a substance having an acceptor property can be used for the hole-injection layer 111. Note that the use of such a substance having a hole-transport property which contains a substance having an acceptor property enables selection of a material used to form an electrode regardless of its work function. In other words, besides a material having a high work function, a material having a low work function can be used for the first electrode 101. Examples of the substance having an acceptor property include 7,7,8,8-tetracyano-2,3,5,6-tetrafluoroquinodimethane (abbreviation: F₄-TCNQ) and chloranil. In addition, transition metal oxides can be given. Moreover, an oxide of metals belonging to Groups 4 to 8 of the periodic table can be used. Specifically, it is preferable to use vanadium oxide, niobium oxide, tantalum oxide, chromium oxide, molybdenum oxide, tungsten oxide, manganese oxide, and rhenium oxide because of their high electron accepting properties. In particular, molybdenum oxide is more preferable because of its stability in the atmosphere, low hygroscopic property, and easiness of handling.

As the substance with a hole-transport property which is used for the composite material, any of a variety of organic compounds such as aromatic amine compounds, carbazole derivatives, aromatic hydrocarbons, and high molecular compounds (e.g., oligomers, dendrimers, or polymers) can be used. Note that the substance with a hole-transport property which is used for the composite material is preferably a substance having a hole mobility of 10⁻⁶ cm²/Vs or more. Organic compounds that can be used as the substance with a hole-transport property in the composite material are specifically given below.

Examples of the aromatic amine compounds that can be used for the composite material are N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), and the like. Specific examples of the carbazole derivatives are 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), 1,4-bis[4-(N-carbazolyl)phenyl]-2,3,5,6-tetraphenylbenzene, and the like. Examples of the aromatic hydrocarbons are 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, and the like. Besides, pentacene, coronene, or the like can also be used. The aromatic hydrocarbons may have a vinyl skeleton. Examples of the aromatic hydrocarbon having a vinyl skeleton are 4,4′-bis(2,2-diphenylvinyl)biphenyl (abbreviation: DPVBi), 9,10-bis[4-(2,2-diphenylvinyl)phenyl]anthracene (abbreviation: DPVPA), and the like. Note that any of the organic compounds of embodiments of the present invention can also be used.

A high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,N-bis(phenyl)benzidine] (abbreviation: poly-TPD) can also be used.

By providing the hole-injection layer, a high hole-injection property can be achieved to allow a light-emitting element to be driven at a low voltage.

Note that the hole-injection layer may be formed of the above-described acceptor material alone or of the above-described acceptor material and another material in combination. In this case, the acceptor material extracts electrons from the hole-transport layer, so that holes can be injected into the hole-transport layer. The acceptor material transfers the extracted electrons to the anode.

The hole-transport layer 112 is a layer containing a substance having a hole-transport property. Examples of the substance having a hole-transport property are aromatic amine compounds such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(spiro-9,9′-bifluoren-2-yl)-N-phenylamino]biphenyl (abbreviation: BSPB), and 4-phenyl-4′-(9-phenylfluoren-9-yl)triphenylamine (abbreviation: BPAFLP). The substances listed here have high hole-transport properties and are mainly ones that have a hole mobility of 10⁻⁶ cm²Ns or higher. An organic compound given as an example of the substance having a hole-transport property in the composite material described above can also be used for the hole-transport layer 112. Moreover, a high molecular compound such as poly(N-vinylcarbazole) (abbreviation: PVK) or poly(4-vinyltriphenylamine) (abbreviation: PVTPA) can also be used. In addition, any of the organic compounds of embodiments of the present invention can also be favorably used. Note that a light-emitting element in which the organic compound of one embodiment of the present invention or the light-emitting element material of one embodiment of the present invention is used for a hole-transport layer can have favorable characteristics. For example, the light-emitting element can have a long lifetime. Alternatively, the light-emitting element can have high emission efficiency.

Note that the layer that contains a substance having a hole-transport property is not limited to a single layer, and may be a stack of two or more layers including any of the above substances. A light-emitting element in which the organic compound of one embodiment of the present invention or the light-emitting element material of one embodiment of the present invention is used for a layer in contact with the light-emitting layer 113 can have favorable characteristics. For example, the light-emitting element can have a long lifetime. Alternatively, the light-emitting element can have high emission efficiency. In particular, the host material in the light-emitting layer is preferably a substance including an anthracene skeleton, or is more preferably a substance including an anthracene skeleton and a carbazole skeleton, in which case the effect is significant. In particular, the substance including an anthracene skeleton or the substance including an anthracene skeleton and a carbazole skeleton is highly suitable as a host material of a blue fluorescent element. A blue fluorescent element in which the organic compound of one embodiment of the present invention or the light-emitting element material of one embodiment of the present invention is used for a hole-transport layer can have favorable characteristics. Specifically, the blue fluorescent element can have high reliability. Alternatively, the blue fluorescent element can have high emission efficiency. Alternatively, the blue fluorescent element can have low driving voltage and low power consumption.

The light-emitting layer 113 may be a layer that emits fluorescence, a layer that emits phosphorescence, or a layer emitting thermally activated delayed fluorescence (TADF). Furthermore, the light-emitting layer 113 may be a single layer or include a plurality of layers containing different light-emitting substances. In the case where the light-emitting layer including a plurality of layers is formed, a layer containing a phosphorescent substance and a layer containing a fluorescent substance may be stacked. In that case, an exciplex described later is preferably utilized for the layer containing the phosphorescent substance.

As the fluorescent substance, any of the following substances can be used, for example. Fluorescent substances other than those given below can also be used.

Examples of the fluorescent substance are 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N-(pyrene-1,6-diyl)bis[(6,N-diphenylbenzo[b]naphtho[1,2-d]furan)-8-amine] (abbreviation: 1,6BnfAPrn-03), N,N′-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), and the like. Fused aromatic diamine compounds typified by pyrenediamine compounds such as 1,6FLPAPm, 1,6mMemFLPAPm, and 1,6BnfAPrn-03 are preferable because of their high hole-trapping properties, high emission efficiency, and high reliability. Note that the organic compounds of embodiments of the present invention are each preferably used as a fluorescent substance. A light-emitting element including any of the organic compounds of embodiments of the present invention can emit blue light with favorable chromaticity and have high external quantum efficiency.

Examples of a material which can be used as a phosphorescent substance in the light-emitting layer 113 are as follows. The examples include organometallic iridium complexes having 4H-triazole skeletons, such as tris{2-[5-(2-methylphenyl)-4-(2,6-dimethylphenyl)-4H-1,2,4-triazol-3-yl-κN²]phenyl-κ C}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); organometallic iridium complexes having 1H-triazole skeletons, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having imidazole skeletons, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic iridium complexes in which a phenylpyridine derivative having an electron-withdrawing group is a ligand, such as bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C²′]iridium(III) picolinate (abbreviation: FIrpic), bis{2-[3′,5′-bis(trifluoromethyl)phenyl]pyridinato-N,C²′ }iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIr(acac)). These are compounds emitting blue phosphorescence and have an emission peak at 440 nm to 520 nm.

Other examples include organometallic iridium complexes having pyrimidine skeletons, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having pyrazine skeletons, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having pyridine skeletons, such as tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), and bis(2-phenylquinolinato-N,C²′)iridium(III) acetylacetonate (abbreviation: [Ir(pq)₂(acac)]); and rare earth metal complexes such as tris(acetylacetonato)(monophenanthroline)terbium(III) (abbreviation: [Tb(acac)₃(Phen)]). These are mainly compounds emitting green phosphorescence and have an emission peak at 500 nm to 600 nm. Note that organometallic iridium complexes having pyrimidine skeletons have distinctively high reliability and emission efficiency and thus are especially preferable.

Other examples include organometallic iridium complexes having pyrimidine skeletons, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having pyrazine skeletons, such as (acetylacetonato)bis(2,3,5-triphenylpyrazinato)iridium(III) (abbreviation: [Ir(tppr)₂(acac)]), bis(2,3,5-triphenylpyrazinato)(dipivaloylhnethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having pyridine skeletons, such as tris(1-phenylisoquinolinato-N,C²′)iridium(III) (abbreviation: [Ir(piq)₃]) and bis(1-phenylisoquinolinato-N,C²′)iridium(III) acetylacetonate (abbreviation: [Ir(piq)₂(acac)]); platinum complexes such as 2,3,7,8,12,13,17,18-octaethyl-21H,23H-porphyrin platinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These are compounds emitting red phosphorescence and have an emission peak at 600 nm to 700 nm. Furthermore, organometallic iridium complexes having pyrazine skeletons can provide red light emission with favorable chromaticity.

As well as the above phosphorescent compounds, a variety of phosphorescent substances may be selected and used.

Examples of the TADF material include a fullerene, a derivative thereof, an acridine derivative such as proflavine, and eosin. Furthermore, a metal-containing porphyrin, such as a porphyrin containing magnesium (Mg), zinc (Zn), cadmium (Cd), tin (Sn), platinum (Pt), indium (In), or palladium (Pd) can be used. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂(OEP)), which are shown in the following structural formulae.

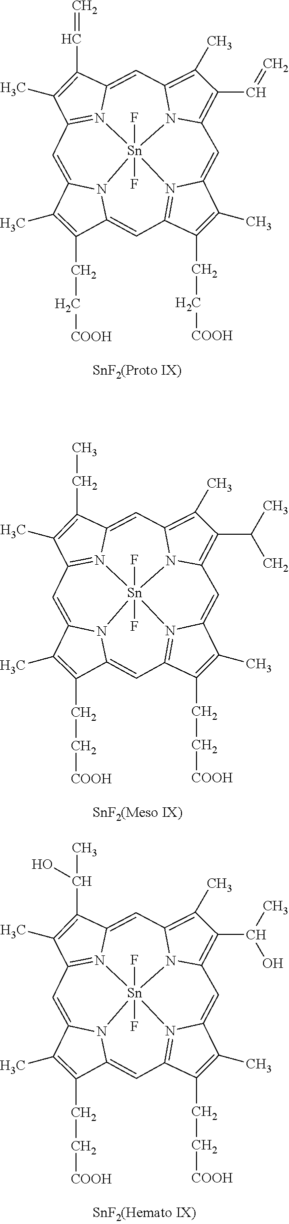

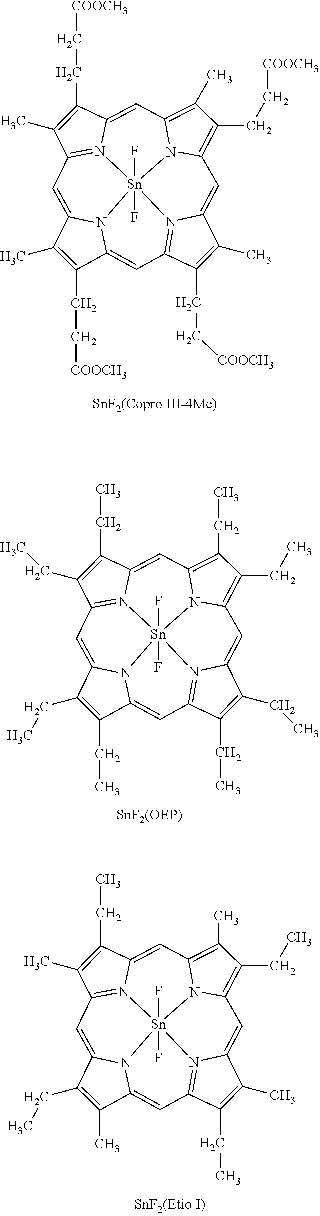

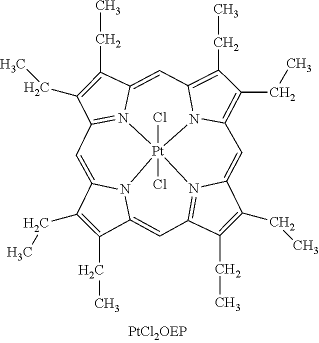

Alternatively, a heterocyclic compound having a π-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazine-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimnethyl-9,10-dihydroacridine)phenyl]sulfone (abbreviation: DMAC-DPS), or 10-phenyl-1 OH, 10′H-spiro[acridin-9,9′-anthracen]-10′-one (abbreviation: ACRSA) shown in the following structural formulae, can be used as the host material 131 composed of one kind of compound. The heterocyclic compound is preferable because of having the π-electron rich heteroaromatic ring and the π-electron deficient heteroaromatic ring, for which the electron-transport property and the hole-transport property are high. Note that a substance in which the π-electron rich heteroaromatic ring is directly bonded to the π-electron deficient heteroaromatic ring is particularly preferably used because the donor property of the π-electron rich heteroaromatic ring and the acceptor property of the π-electron deficient heteroaromatic ring are both increased, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring.

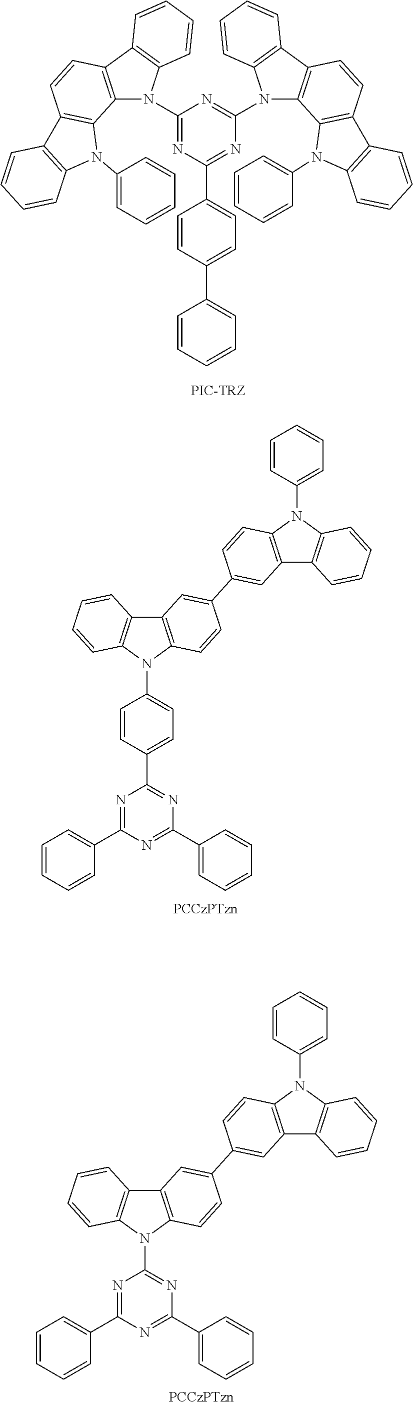 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

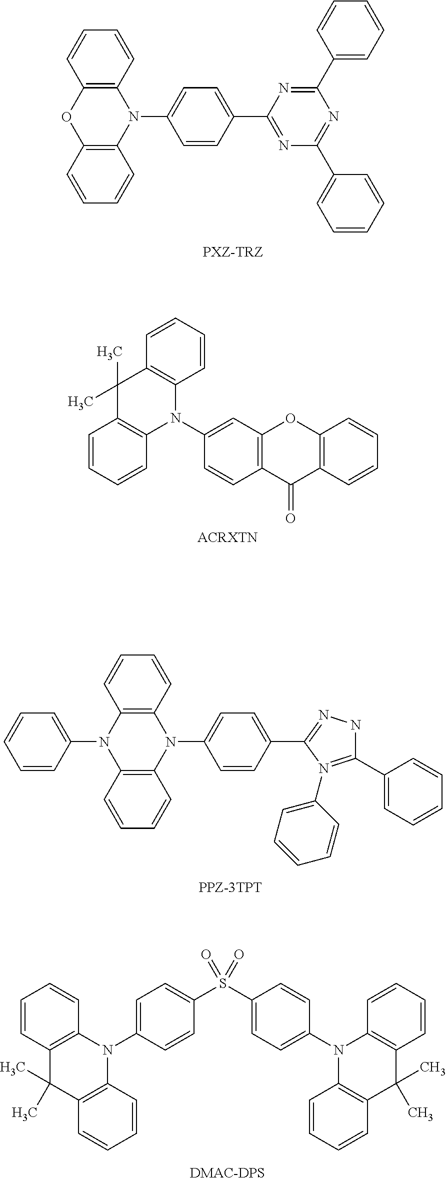

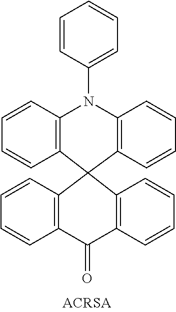 [SMILES: O=C1c2ccccc2C2(c3ccccc31)c1ccccc1N(c1ccccc1)c1ccccc12]

As a host material of the light-emitting layer, various carrier-transport materials, such as a material with an electron-transport property or a material with a hole-transport property, can be used.

Examples of the material with an electron-transport property are a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); a heterocyclic compound having a triazole skeleton such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II); a heterocyclic compound having a diazine skeleton such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), or 4,6-bis[3-(4-dibenzothienyl)phenyl]pyrimidine (abbreviation: 4,6mDBTP2Pm-II); and a heterocyclic compound having a pyridine skeleton such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB). Among the above materials, a heterocyclic compound having a diazine skeleton and a heterocyclic compound having a pyridine skeleton have high reliability and are thus preferable. Specifically, a heterocyclic compound having a diazine (pyrimidine or pyrazine) skeleton has a high electron-transport property to contribute to a reduction in drive voltage.

Note that the organic compounds of embodiments of the present invention can be used as the material having an electron-transport property. In particular, the organic compound including a fused polycyclic aromatic hydrocarbon skeleton, which is one embodiment of the present invention, has a high electron-transport property and thus has a bipolar property; therefore, the organic compound can be suitably used as a host material. The organic compound including of one embodiment of the present invention which has an anthracene skeleton as the fused polycyclic aromatic hydrocarbon skeleton particularly has a high electron-transport property and can be suitably used as a host material and as a material of an electron-transport layer.

Examples of the material having a hole-transport property include a compound having an aromatic amine skeleton, such as 4,4′-bis[N-(1-naphthyl)-N-phenylamnino]biphenyl (abbreviation: NPB), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]spiro-9,9′-bifluoren-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above materials, a compound having an aromatic amine skeleton and a compound having a carbazole skeleton are preferable because these compounds are highly reliable and have high hole-transport properties to contribute to a reduction in drive voltage. Hole-transport materials can be selected from a variety of substances as well as from the hole-transport materials given above.

Note that the organic compound of one embodiment of the present invention may be used as the material having a hole-transport property because it has a high hole-transport property.

In the case of using a fluorescent substance as a light-emitting substance, materials having an anthracene skeleton such as 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), and 9-phenyl-10-{4-(9-phenyl-9H-fluoren-9-yl)-biphenyl-4′-yl}-anthracene (abbreviation: FLPPA) are preferably used as host materials. The use of a substance having an anthracene skeleton as the host material for the fluorescent substance makes it possible to obtain a light-emitting layer with high emission efficiency and high durability. In particular, CzPA, cgDBCzPA, 2mBnfPPA, and PCzPA are preferable because of their excellent characteristics.

Note that the host material may be a mixture of a plurality of kinds of substances, and in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The ratio of the content of the material having a hole-transport property to the content of the material having an electron-transport property may be 1:9 to 9:1.

These mixed host materials may form an exciplex. When a combination of these materials is selected so as to form an exciplex that exhibits light emission whose wavelength overlaps the wavelength of a lowest-energy-side absorption band of the fluorescent substance, the phosphorescent substance, or the TADF material, energy is transferred smoothly and light emission can be obtained efficiently. Such a structure is preferable in that drive voltage can be reduced.

The light-emitting layer 113 having the above-described structure can be formed by co-evaporation by a vacuum evaporation method, or a gravure printing method, an offset printing method, an inkjet method, a spin coating method, a dip coating method, or the like using a mixed solution.

The electron-transport layer 114 is a layer including a substance having an electron-transport property. As a substance having an electron-transport property, the materials having an electron-transport property or having an anthracene skeleton, which are described above as materials for the host material, can be used.

Between the electron-transport layer and the light-emitting layer, a layer that controls transport of electron carriers may be provided. This is a layer formed by addition of a small amount of a substance having a high electron-trapping property to the aforementioned material having a high electron-transport property, and the layer is capable of adjusting carrier balance by retarding transport of electron carriers. Such a structure is very effective in preventing a problem (such as a reduction in element lifetime) caused when electrons pass through the light-emitting layer.

In addition, the electron-injection layer 115 may be provided in contact with the second electrode 102 between the electron-transport layer 114 and the second electrode 102. For the electron-injection layer 115, an alkali metal, an alkaline earth metal, or a compound thereof, such as lithium fluoride (LiF), cesium fluoride (CsF), or calcium fluoride (CaF₂), can be used. For example, a layer that is formed using a substance having an electron-transport property and contains an alkali metal, an alkaline earth metal, or a compound thereof can be used. In addition, an electride may be used for the electron-injection layer 115. Examples of the electride include a substance in which electrons are added at high concentration to calcium oxide-aluminum oxide. Note that a layer that is formed using a substance having an electron-transport property and contains an alkali metal or an alkaline earth metal is preferably used as the electron-injection layer 115, in which case electron injection from the second electrode 102 is efficiently performed.

Instead of the electron-injection layer 115, a charge-generation layer 116 may be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film containing the above-described acceptor material as a material included in the composite material and a film containing the above-described hole-transport material. When a potential is applied to the p-type layer 117, electrons are injected into the electron-transport layer 114 and holes are injected into the second electrode 102 serving as a cathode; thus, the light-emitting element operates. When a layer containing the organic compound of one embodiment of the present invention exists in the electron-transport layer 114 so as to be in contact with the charge-generation layer 116, a luminance decrease due to accumulation of driving time of the light-emitting element can be suppressed, and thus, the light-emitting element can have a long lifetime.

Note that the charge-generation layer 116 preferably includes either an electron-relay layer 118 or an electron-injection buffer layer 119 or both in addition to the p-type layer 117.

The electron-relay layer 118 contains at least the substance with an electron-transport property and has a function of preventing an interaction between the electron-injection buffer layer 119 and the p-type layer 117 and smoothly transferring electrons. The LUMO level of the substance with an electron-transport property contained in the electron-relay layer 118 is preferably between the LUMO level of an acceptor substance in the p-type layer 117 and the LUMO level of a substance contained in a layer of the electron-transport layer 114 in contact with the charge-generation layer 116. As a specific value of the energy level, the LUMO level of the substance with an electron-transport property contained in the electron-relay layer 118 is preferably higher than or equal to −5.0 eV, further preferably higher than or equal to −5.0 eV and lower than or equal to −3.0 eV. Note that as the substance with an electron-transport property in the electron-relay layer 118, a phthalocyanine-based material or a metal complex having a metal-oxygen bond and an aromatic ligand is preferably used.

A substance having a high electron-injection property can be used for the electron-injection buffer layer 119. For example, an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)) can be used.

In the case where the electron-injection buffer layer 119 contains the substance having an electron-transport property and a donor substance, an organic compound such as tetrathianaphthacene (abbreviation: TTN), nickelocene, or decamethylnickelocene can be used as the donor substance, as well as an alkali metal, an alkaline earth metal, a rare earth metal, a compound of the above metal (e.g., an alkali metal compound (including an oxide such as lithium oxide, a halide, and a carbonate such as lithium carbonate or cesium carbonate), an alkaline earth metal compound (including an oxide, a halide, and a carbonate), and a rare earth metal compound (including an oxide, a halide, and a carbonate)). Note that as the substance having an electron-transport property, a material similar to the above-described material used for the electron-transport layer 114 can be used. Furthermore, the organic compound of the present invention can be used.

For the second electrode 102, any of metals, alloys, electrically conductive compounds, and mixtures thereof which have a low work function (specifically, a work function of 3.8 eV or less) or the like can be used. Specific examples of such a cathode material are elements belonging to Groups 1 and 2 of the periodic table, such as alkali metals (e.g., lithium (Li) and cesium (Cs)), magnesium (Mg), calcium (Ca), and strontium (Sr), alloys thereof (e.g., MgAg and AlLi), rare earth metals such as europium (Eu) and ytterbium (Yb), alloys thereof, and the like. However, when the electron-injection layer is provided between the second electrode 102 and the electron-transport layer, for the second electrode 102, any of a variety of conductive materials such as Al, Ag, ITO, or indium oxide-tin oxide containing silicon or silicon oxide can be used regardless of the work function. Films of these conductive materials can be formed by a dry method such as a vacuum evaporation method or a sputtering method, an inkjet method, a spin coating method, or the like. In addition, the films of these conductive materials may be formed by a wet method using a sol-gel method, or by a wet method using paste of a metal material.

Any of a variety of methods can be used to form the EL layer 103 regardless of whether it is a dry process or a wet process. For example, a vacuum evaporation method, a gravure printing method, an offset printing method, a screen printing method, an inkjet method, a spin coating method, or the like may be used.

In addition, the electrode may be formed by a wet method using a sol-gel method, or by a wet method using paste of a metal material. Alternatively, the electrode may be formed by a dry method such as a sputtering method or a vacuum evaporation method.

Light emission from the light-emitting element is extracted out through one or both of the first electrode 101 and the second electrode 102. Therefore, one or both of the first electrode 101 and the second electrode 102 are formed with a light-transmitting conductive material.

The structure of the layers provided between the first electrode 101 and the second electrode 102 is not limited to the above-described structure. Preferably, a light-emitting region where holes and electrons recombine is positioned away from the first electrode 101 and the second electrode 102 so that quenching due to the proximity of the light-emitting region and a metal used for electrodes and carrier-injection layers can be prevented.

Furthermore, in order that transfer of energy from an exciton generated in the light-emitting layer can be suppressed, preferably, the hole-transport layer and the electron-transport layer which are in contact with the light-emitting layer 113, particularly a carrier-transport layer in contact with a side closer to the recombination region in the light-emitting layer 113, are formed using a substance having a wider band gap than the light-emitting substance of the light-emitting layer or the emission center substance included in the light-emitting layer.

Next, a mode of a light-emitting element with a structure in which a plurality of light-emitting units are stacked (this type of light-emitting element is also referred to as a stacked element) is described with reference to FIG. 1C. This light-emitting element includes a plurality of light-emitting units between an anode and a cathode. One light-emitting unit has the same structure as the EL layer 103 illustrated in FIG. 1A. In other words, the light-emitting element illustrated in FIG. 1A or FIG. 1B includes a single light-emitting unit, and the light-emitting element illustrated in FIG. 1C includes a plurality of light-emitting units.

In FIG. 1C, a first light-emitting unit 511 and a second light-emitting unit 512 are stacked between a first electrode 501 and a second electrode 502, and a charge-generation layer 513 is provided between the first light-emitting unit 511 and the second light-emitting unit 512. The first electrode 501 and the second electrode 502 correspond, respectively, to the first electrode 101 and the second electrode 102 illustrated in FIG. 1A, and the materials given in the description for FIG. 1A can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when a voltage is applied between the first electrode 501 and the second electrode 502. That is, in FIG. 1C, the charge-generation layer 513 injects electrons into the first light-emitting unit 511 and holes into the second light-emitting unit 512 when a voltage is applied so that the potential of the first electrode becomes higher than the potential of the second electrode.

The charge-generation layer 513 preferably has a structure similar to the structure of the charge-generation layer 116 described with reference to FIG. 1B. Since the composite material of an organic compound and a metal oxide is superior in carrier-injection property and carrier-transport property, low-voltage driving or low-current driving can be achieved. Note that when a surface of a light-emitting unit on the anode side is in contact with the charge-generation layer 513, the charge-generation layer 513 can also serve as a hole-injection layer of the light-emitting unit; thus, a hole-injection layer is not necessarily formed in the light-emitting unit.

In the case where the electron-injection buffer layer 119 is provided, the electron-injection buffer layer serves as the electron-injection layer in the light-emitting unit on the anode side and the light-emitting unit does not necessarily further need an electron-injection layer.

Note that when a layer in contact with a surface of the charge-generation layer 513 on the anode side in a light-emitting unit (typically, the electron-transport layer in the light-emitting unit on the anode side) contains the organic compound of one embodiment of the present invention, a luminance decrease due to accumulation of driving time can be suppressed, and thus, the light-emitting element can have high reliability.

The light-emitting element having two light-emitting units is described with reference to FIG. 1C; however, one embodiment of the present invention can be similarly applied to a light-emitting element in which three or more light-emitting units are stacked. With a plurality of light-emitting units partitioned by the charge-generation layer 513 between a pair of electrodes as in the light-emitting element according to this embodiment, it is possible to provide an element which can emit light with high luminance with the current density kept low and has a long lifetime. A light-emitting device that can be driven at a low voltage and has low power consumption can be realized.

Furthermore, when emission colors of the light-emitting units are made different, light emission of a desired color can be obtained from the light-emitting element as a whole. For example, it is easy to enable a light-emitting element having two light-emitting units to emit white light as the whole element when the emission colors of the first light-emitting unit are red and green and the emission color of the second light-emitting unit is blue.

### <<Micro Optical Resonator (Microcavity) Structure>>

A light-emitting element with a microcavity structure is formed with the use of a reflective electrode and a semi-transmissive and semi-reflective electrode as the pair of electrodes. The reflective electrode and the semi-transmissive and semi-reflective electrode correspond to the first electrode and the second electrode described above. The light-emitting element with a microcavity structure includes at least an EL layer between the reflective electrode and the semi-transmissive and semi-reflective electrode. The EL layer includes at least a light-emitting layer serving as a light-emitting region.

Light emitted from the light-emitting layer included in the EL layer is reflected and resonated by the reflective electrode and the semi-transmissive and semi-reflective electrode. Note that the reflective electrode has a visible light reflectivity of 40% to 100%, preferably 70% to 100% and a resistivity of 1×10⁻² Ωcm or lower. In addition, the semi-transmissive and semi-reflective electrode has a visible light reflectivity of 20% to 80%, preferably 40% to 70%, and a resistivity of 1×10⁻² Ωcm or lower.

In the light-emitting element, by changing thicknesses of the transparent conductive film, the composite material, the carrier-transport material, and the like, the optical path length between the reflective electrode and the semi-transmissive and semi-reflective electrode can be changed. Thus, light with a wavelength that is resonated between the reflective electrode and the semi-transmissive and semi-reflective electrode can be intensified while light with a wavelength that is not resonated therebetween can be attenuated.

Note that light that is emitted from the light-emitting layer and reflected back by the reflective electrode (first reflected light) considerably interferes with light that directly enters the semi-transmissive and semi-reflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of color to be amplified). In that case, the phases of the first reflected light and the first incident light can be aligned with each other and the light emitted from the light-emitting layer can be further amplified.

Note that in the above structure, the EL layer may be formed of light-emitting layers or may be a single light-emitting layer. The tandem light-emitting element described above may be combined with the EL layers; for example, a light-emitting element may have a structure in which a plurality of EL layers is provided, a charge-generation layer is provided between the EL layers, and each EL layer is formed of light-emitting layers or a single light-emitting layer.

### <<Light-Emitting Device>>

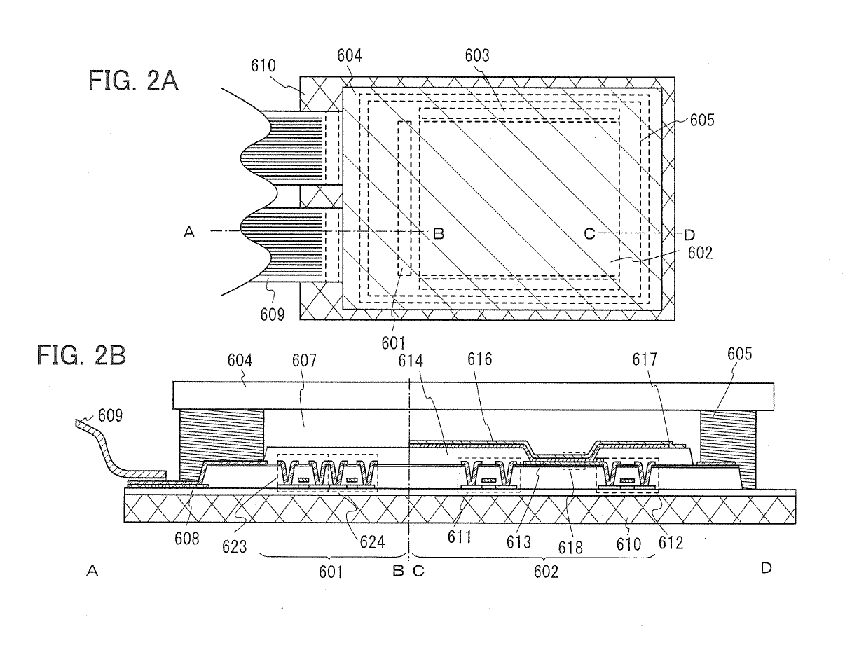

A light-emitting device of one embodiment of the present invention is described using FIGS. 2A and 2B. Note that FIG. 2A is a top view illustrating the light-emitting device and FIG. 2B is a cross-sectional view of FIG. 2A taken along lines A-B and C-D. This light-emitting device includes a driver circuit portion (source line driver circuit) 601, a pixel portion 602, and a driver circuit portion (gate line driver circuit) 603, which can control light emission of a light-emitting element and illustrated with dotted lines. A reference numeral 604 denotes a sealing substrate; 605, a sealing material; and 607, a space surrounded by the sealing material 605.

Reference numeral 608 denotes a wiring for transmitting signals to be input to the source line driver circuit 601 and the gate line driver circuit 603 and receiving signals such as a video signal, a clock signal, a start signal, and a reset signal from a flexible printed circuit (FPC) 609 serving as an external input terminal. Although only the FPC is illustrated here, a printed wiring board (PWB) may be attached to the FPC. The light-emitting device in the present specification includes, in its category, not only the light-emitting device itself but also the light-emitting device provided with the FPC or the PWB.

Next, a cross-sectional structure will be described with reference to FIG. 2B. The driver circuit portion and the pixel portion are formed over an element substrate 610; the source line driver circuit 601, which is a driver circuit portion, and one of the pixels in the pixel portion 602 are illustrated here.

As the source line driver circuit 601, a CMOS circuit in which an n-channel FET 623 and a p-channel FET 624 are combined is formed. In addition, the driver circuit may be formed with any of a variety of circuits such as a CMOS circuit, a PMOS circuit, or an NMOS circuit. Although a driver integrated type in which the driver circuit is formed over the substrate is described in this embodiment, the driver circuit is not necessarily formed over the substrate, and the driver circuit can be formed outside, not over the substrate.

The pixel portion 602 includes a plurality of pixels including a switching FET 611, a current controlling FET 612, and a first electrode 613 electrically connected to a drain of the current controlling FET 612. One embodiment of the present invention is not limited to the structure. The pixel portion may include three or more FETs and a capacitor in combination.

The kind and crystallinity of a semiconductor used for the FETs is not particularly limited; an amorphous semiconductor or a crystalline semiconductor may be used. Examples of the semiconductor used for the FETs include Group 13 semiconductors, Group 14 semiconductors, compound semiconductors, oxide semiconductors, and organic semiconductor materials. Oxide semiconductors are particularly preferable. Examples of the oxide semiconductor include an In—Ga oxide and an In-M-Zn oxide (M is Al, Ga, Y, Zr, La, Ce, or Nd). Note that an oxide semiconductor material that has an energy gap of 2 eV or more, preferably 2.5 eV or more, further preferably 3 eV or more is preferably used, in which case the off-state current of the transistors can be reduced.

Note that to cover an end portion of the first electrode 613, an insulator 614 is formed. The insulator 614 can be formed using a positive photosensitive acrylic resin film here.

The insulator 614 is formed to have a curved surface with curvature at its upper or lower end portion in order to obtain favorable coverage. For example, in the case where positive photosensitive acrylic is used for a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a second electrode 617 are formed over the first electrode 613. The first electrode 613, the EL layer 616, and the second electrode 617 correspond, respectively, to the first electrode 101, the EL layer 103, and the second electrode 102 in FIG. 1A or 1B, and correspond, respectively, to the first electrode 501, the EL layer 503, and the second electrode 502 in FIG. 1C.

The EL layer 616 preferably includes the organic compound of one embodiment of the present invention. The organic compound is preferably used as a light-emitting substance, a hole-transport material, a host material, or an assist material in a light-emitting layer.

The sealing substrate 604 is attached to the element substrate 610 with the sealing material 605, so that a light-emitting element 618 is provided in the space 607 surrounded by the element substrate 610, the sealing substrate 604, and the sealing material 605. The space 607 is filled with a filler, and may be filled with an inert gas (such as nitrogen or argon) or the sealing material 605. It is preferable that the sealing substrate 604 be provided with a recessed portion and a drying agent be provided in the recessed portion, in which case deterioration due to influence of moisture can be suppressed.

An epoxy-based resin or glass frit is preferably used for the sealing material 605. It is preferable that such a material do not transmit moisture or oxygen as much as possible. As the element substrate 610 and the sealing substrate 604, a glass substrate, a quartz substrate, or a plastic substrate formed of fiber reinforced plastic (FRP), polyvinyl fluoride (PVF), polyester, or acrylic can be used.

Note that in this specification and the like, a transistor or a light-emitting element can be formed using any of a variety of substrates, for example. The type of a substrate is not limited to a certain type. As the substrate, a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, a base material film, or the like can be used, for example. As an example of a glass substrate, a barium borosilicate glass substrate, an aluminoborosilicate glass substrate, a soda lime glass substrate, or the like can be given. Examples of the flexible substrate, the attachment film, the base material film, and the like are substrates of plastics typified by polyethylene terephthalate (PET), polyethylene naphthalate (PEN), and polyether sulfone (PES). Another example is a synthetic resin such as acrylic. Alternatively, polytetrafluoroethylene (PTFE), polypropylene, polyester, polyvinyl fluoride, polyvinyl chloride, or the like can be used. Alternatively, polyamide, polyimide, aramid, epoxy, an inorganic vapor deposition film, paper, or the like can be used. Specifically, the use of semiconductor substrates, single crystal substrates, SOI substrates, or the like enables the manufacture of small-sized transistors with a small variation in characteristics, size, shape, or the like and with high current capability. A circuit using such transistors achieves lower power consumption of the circuit or higher integration of the circuit.

Alternatively, a flexible substrate may be used as the substrate, and the transistor or the light-emitting element may be provided directly on the flexible substrate. Still alternatively, a separation layer may be provided between the substrate and the transistor or the substrate and the light-emitting element. The separation layer can be used when part or the whole of a semiconductor device formed over the separation layer is separated from the substrate and transferred onto another substrate. In such a case, the transistor can be transferred to a substrate having low heat resistance or a flexible substrate. For the separation layer, a stack including inorganic films, which are a tungsten film and a silicon oxide film, or an organic resin film of polyimide or the like formed over a substrate can be used, for example.

In other words, a transistor or a light-emitting element may be formed using one substrate, and then transferred to another substrate. Examples of a substrate to which a transistor or a light-emitting element is transferred include, in addition to the above-described substrates over which transistors can be formed, a paper substrate, a cellophane substrate, an aramid film substrate, a polyimide film substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupra, rayon, or regenerated polyester), or the like), a leather substrate, and a rubber substrate. When such a substrate is used, a transistor with excellent characteristics or a transistor with low power consumption can be formed, a device with high durability or high heat resistance can be provided, or reduction in weight or thickness can be achieved.

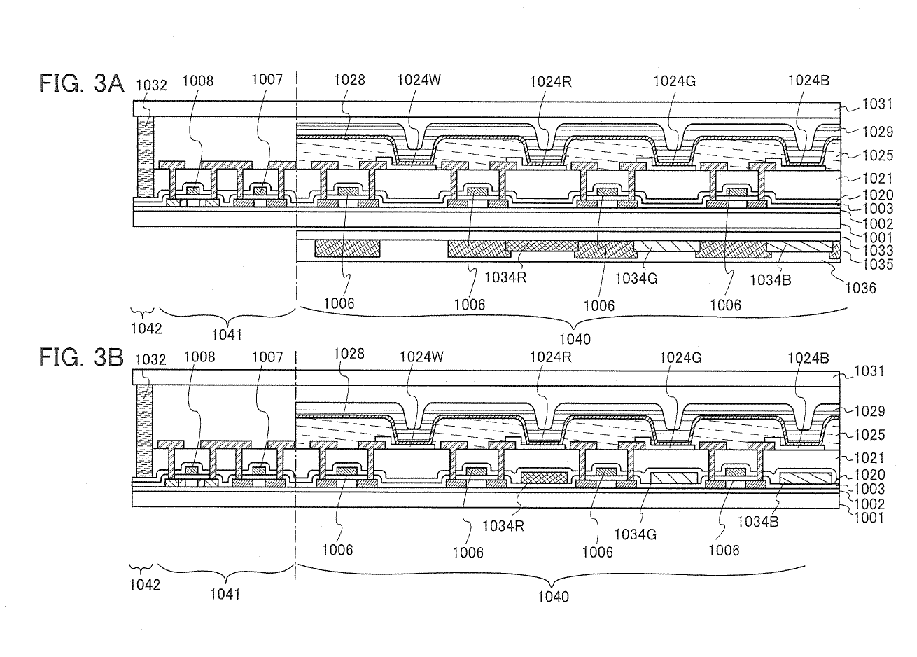

FIGS. 3A and 3B each illustrate an example of a light-emitting device in which full color display is achieved by formation of a light-emitting element exhibiting white light emission and with the use of coloring layers (color filters) and the like. In FIG. 3A, a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of light-emitting elements, a partition 1025, an EL layer 1028, a second electrode 1029 of the light-emitting elements, a sealing substrate 1031, a sealing material 1032, and the like are illustrated.

In FIG. 3A, coloring layers (a red coloring layer 1034R, a green coloring layer 1034G, and a blue coloring layer 1034B) are provided on a transparent base material 1033. A black layer (a black matrix) 1035 may be additionally provided. The transparent base material 1033 provided with the coloring layers and the black layer is positioned and fixed to the substrate 1001. Note that the coloring layers and the black layer are covered with an overcoat layer 1036. In FIG. 3A, light emitted from part of the light-emitting layer does not pass through the coloring layers, while light emitted from the other part of the light-emitting layer passes through the coloring layers. Since light which does not pass through the coloring layers is white and light which passes through any one of the coloring layers is red, blue, or green, an image can be displayed using pixels of the four colors.

Note that a light-emitting element including the organic compound of one embodiment of the present invention as a light-emitting substance can have high emission efficiency and low power consumption.

FIG. 3B illustrates an example in which the coloring layers (the red coloring layer 1034R, the green coloring layer 1034G, and the blue coloring layer 1034B) are provided between the gate insulating film 1003 and the first interlayer insulating film 1020. As in the structure, the coloring layers may be provided between the substrate 1001 and the sealing substrate 1031.

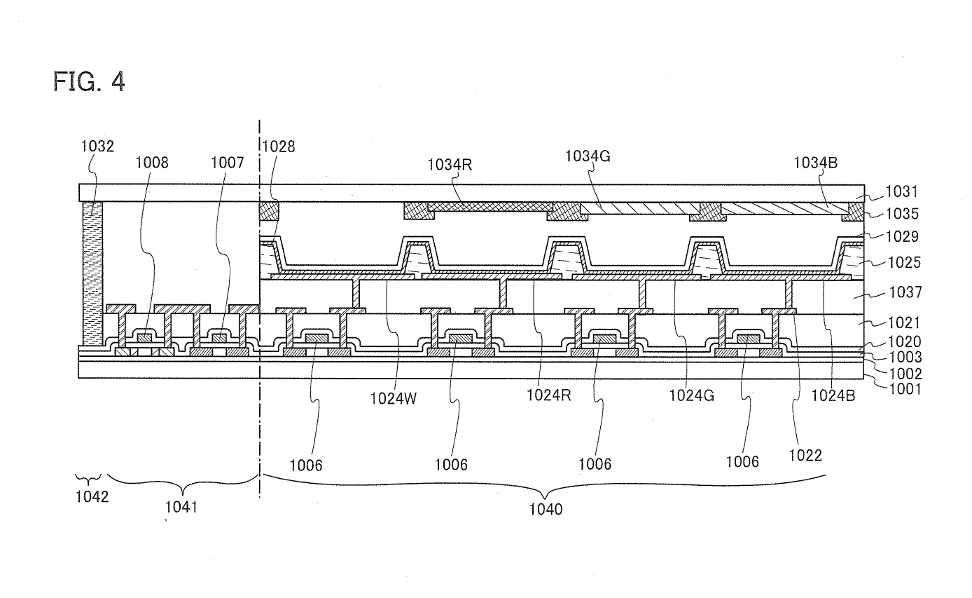

The above-described light-emitting device is a light-emitting device having a structure in which light is extracted from the substrate 1001 side where the FETs are formed (a bottom emission structure), but may be a light-emitting device having a structure in which light is extracted from the sealing substrate 1031 side (a top emission structure). FIG. 4 is a cross-sectional view of a light-emitting device having a top emission structure. In this case, a substrate which does not transmit light can be used as the substrate 1001. The process up to the step of forming a connection electrode which connects the FET and the anode of the light-emitting element is performed in a manner similar to that of the light-emitting device having a bottom emission structure. Then, a third interlayer insulating film 1037 is formed to cover an electrode 1022. This insulating film may have a planarization function. The third interlayer insulating film 1037 can be formed using a material similar to that of the second interlayer insulating film, and can alternatively be formed using any of various materials.

The first electrodes 1024W, 1024R, 1024G, and 1024B of the light-emitting elements each serve as an anode here, but may serve as a cathode. Furthermore, in the case of a light-emitting device having a top emission structure as illustrated in FIG. 4, the first electrodes are preferably reflective electrodes. The EL layer 1028 is formed to have a structure similar to the structure of the EL layer 103 in FIG. 1A or 1B or the EL layer 503 in FIG. 1C, with which white light emission can be obtained.

In the case of a top emission structure as illustrated in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black layer (black matrix) 1035 which is positioned between pixels. The coloring layers (the red coloring layer 1034R, the green coloring layer 1034G, and the blue coloring layer 1034B) and the black layer may be covered with the overcoat layer. Note that a light-transmitting substrate is used as the sealing substrate 1031.

Although an example in which full color display is performed using four colors of red, green, blue, and white is shown here, there is no particular limitation and full color display using three colors of red, green, and blue or four colors of red, green, blue, and yellow may be performed.

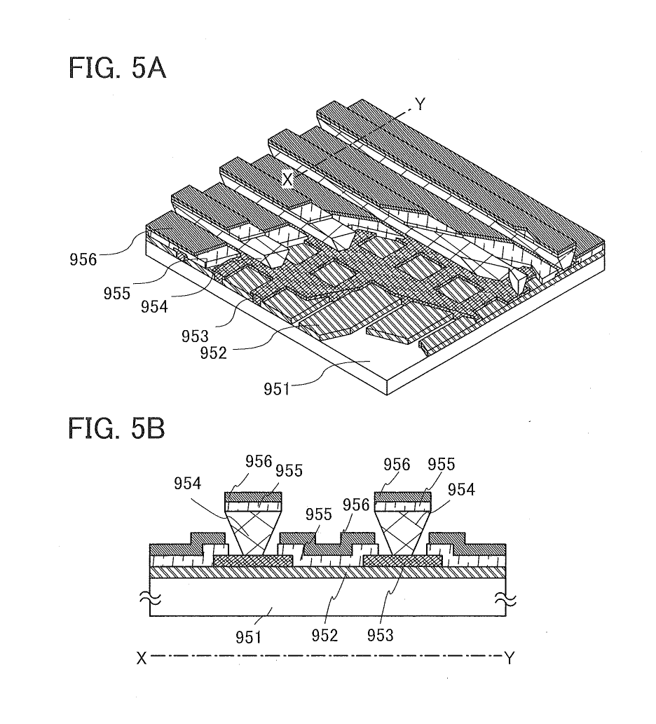

FIGS. 5A and 5B illustrate a passive matrix light-emitting device that is one embodiment of the present invention. FIG. 5A is a perspective view of the light-emitting device, and FIG. 5B is a cross-sectional view of FIG. 5A taken along line X-Y. In FIGS. 5A and 5B, an EL layer 955 is provided between an electrode 952 and an electrode 956 over a substrate 951. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side which is in the same direction as a plane direction of the insulating layer 953 and in contact with the insulating layer 953) is shorter than the upper side (a side which is in the same direction as the plane direction of the insulating layer 953 and not in contact with the insulating layer 953). The partition layer 954 thus provided can prevent defects in the light-emitting element due to static electricity or the like.

Since many minute light-emitting elements arranged in a matrix can each be controlled with the FETs formed in the pixel portion, the above-described light-emitting device can be suitably used as a display device for displaying images.

### <<Lighting Device>>

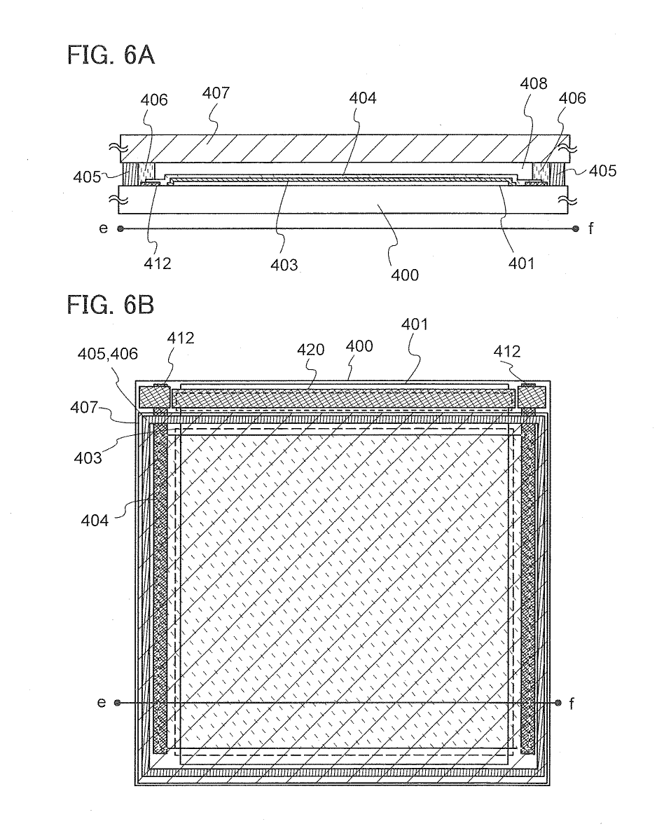

A lighting device that is one embodiment of the present invention is described with reference to FIGS. 6A and 6B. FIG. 6B is a top view of the lighting device, and FIG. 6A is a cross-sectional view of FIG. 6B taken along line e-f.

In the lighting device, a first electrode 401 is formed over a substrate 400 which is a support and has a light-transmitting property. The first electrode 401 corresponds to the first electrode 101 in FIGS. 1A and 1B. When light is extracted through the first electrode 401 side, the first electrode 401 is formed using a material having a light-transmitting property.

A pad 412 for applying a voltage to a second electrode 404 is provided over the substrate 400.

An EL layer 403 is formed over the first electrode 401. The EL layer 403 corresponds to, for example, the EL layer 103 in FIG. 1A or FIG. 1B or the EL layer 503 in FIG. 1C. Refer to the descriptions for the structure.

The second electrode 404 is formed to cover the EL layer 403. The second electrode 404 corresponds to the second electrode 102 in FIG. 1A. The second electrode 404 contains a material having high reflectivity when light is extracted through the first electrode 401 side. The second electrode 404 is connected to the pad 412, whereby a voltage is applied.

A light-emitting element is formed with the first electrode 401, the EL layer 403, and the second electrode 404. The light-emitting element is fixed to a sealing substrate 407 with sealing materials 405 and 406 and sealing is performed, whereby the lighting device is completed. It is possible to use only either the sealing material 405 or the sealing material 406. In addition, the inner sealing material 406 (not shown in FIG. 6B) can be mixed with a desiccant, whereby moisture is adsorbed and the reliability is increased.

When parts of the pad 412 and the first electrode 401 are extended to the outside of the sealing materials 405 and 406, the extended parts can serve as external input terminals. An IC chip 420 mounted with a converter or the like may be provided over the external input terminals.

### <<Electronic Device>>

Examples of an electronic device that is one embodiment of the present invention are described. Examples of the electronic device are television devices (also referred to as TV or television receivers), monitors for computers and the like, cameras such as digital cameras and digital video cameras, digital photo frames, mobile phones (also referred to as cell phones or mobile phone devices), portable game machines, portable information terminals, audio playback devices, and large game machines such as pachinko machines. Specific examples of these electronic devices are given below.

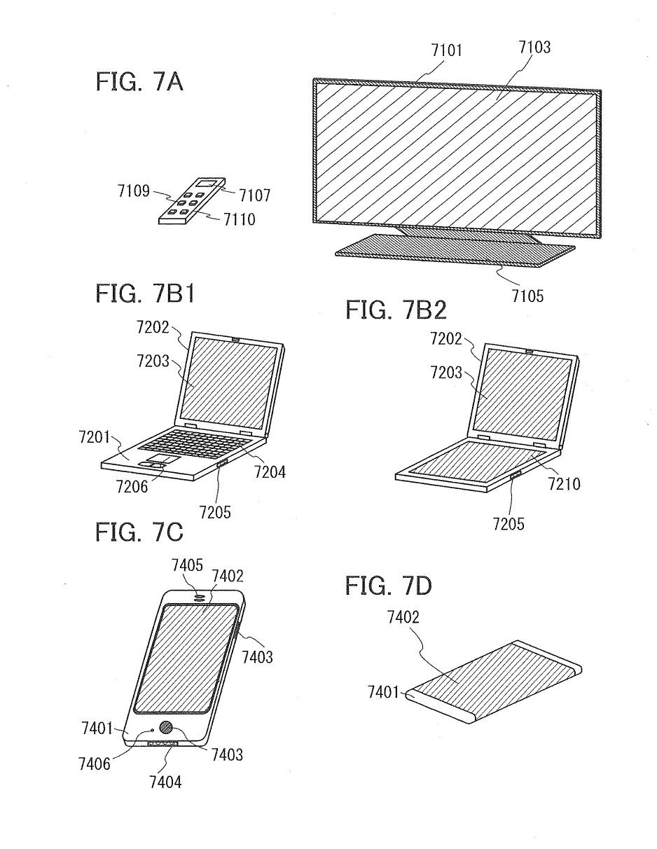

FIG. 7A illustrates an example of a television device. In the television device, a display portion 7103 is incorporated in a housing 7101. In addition, here, the housing 7101 is supported by a stand 7105. Images can be displayed on the display portion 7103, and in the display portion 7103, light-emitting elements are arranged in a matrix.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device is provided with a receiver, a modem, and the like. With the use of the receiver, general television broadcasting can be received. Moreover, when the television device is connected to a communication network with or without wires via the modem, one-way (from a sender to a receiver) or two-way (between a sender and a receiver or between receivers) information communication can be performed.

FIG. 7B1 illustrates a computer, which includes a main body 7201, a housing 7202, a display portion 7203, a keyboard 7204, an external connection port 7205, a pointing device 7206, and the like. Note that this computer is manufactured by using light-emitting elements arranged in a matrix in the display portion 7203. The computer illustrated in FIG. 7B1 may have a structure illustrated in FIG. 7B2. A computer illustrated in FIG. 7B2 is provided with a second display portion 7210 instead of the keyboard 7204 and the pointing device 7206. The second display portion 7210 is a touchscreen, and input can be performed by operation of display for input on the second display portion 7210 with a finger or a dedicated pen. The second display portion 7210 can also display images other than the display for input. The display portion 7203 may also be a touchscreen. Connecting the two screens with a hinge can prevent troubles; for example, the screens can be prevented from being cracked or broken while the computer is being stored or carried.

FIGS. 7C and 7D illustrate an example of a portable information terminal. The portable information terminal is provided with a display portion 7402 incorporated in a housing 7401, operation buttons 7403, an external connection port 7404, a speaker 7405, a microphone 7406, and the like. Note that the portable information terminal has the display portion 7402 including light-emitting elements arranged in a matrix.

Information can be input to the portable information terminal illustrated in FIGS. 7C and 7D by touching the display portion 7402 with a finger or the like. In this case, operations such as making a call and creating an e-mail can be performed by touching the display portion 7402 with a finger or the like.

There are mainly three screen modes of the display portion 7402. The first mode is a display mode mainly for displaying an image. The second mode is an input mode mainly for inputting information such as characters. The third mode is a display-and-input mode in which two modes of the display mode and the input mode are combined.

For example, in the case of making a call or creating an e-mail, a text input mode mainly for inputting text is selected for the display portion 7402 so that text displayed on a screen can be input. In this case, it is preferable to display a keyboard or number buttons on almost the entire screen of the display portion 7402.

When a detection device including a sensor such as a gyroscope or an acceleration sensor for sensing inclination is provided inside the mobile phone, screen display of the display portion 7402 can be automatically changed by determining the orientation of the mobile phone (whether the mobile phone is placed horizontally or vertically).

The screen modes are switched by touch on the display portion 7402 or operation with the operation buttons 7403 of the housing 7401. The screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal detected by an optical sensor in the display portion 7402 is detected, the screen mode may be controlled so as to be switched from the input mode to the display mode.

The display portion 7402 may function as an image sensor. For example, an image of a palm print, a fingerprint, or the like is taken by the display portion 7402 while in touch with the palm or the finger, whereby personal authentication can be performed. Furthermore, by providing a backlight or a sensing light source which emits near-infrared light in the display portion, an image of a finger vein, a palm vein, or the like can be taken.

Note that in the above electronic devices, any of the structures described in this specification can be combined as appropriate.

The display portion preferably includes a light-emitting element including the organic compound of one embodiment of the present invention. The light-emitting element can have high emission efficiency. Furthermore, the light-emitting element can be driven at low voltage. Thus, the electronic device including the organic compound of one embodiment of the present invention can have low power consumption.

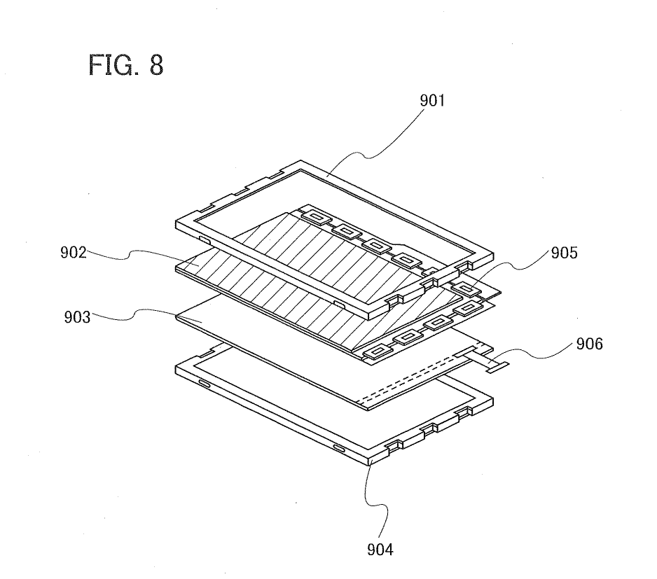

FIG. 8 illustrates an example of a liquid crystal display device including the light-emitting element for a backlight. The liquid crystal display device illustrated in FIG. 8 includes a housing 901, a liquid crystal layer 902, a backlight unit 903, and a housing 904. The liquid crystal layer 902 is connected to a driver IC 905. The light-emitting element is used for the backlight unit 903, to which current is supplied through a terminal 906.

As the light-emitting element, a light-emitting element including the organic compound of one embodiment of the present invention is preferably used. By including the light-emitting element, the backlight of the liquid crystal display device can have low power consumption.

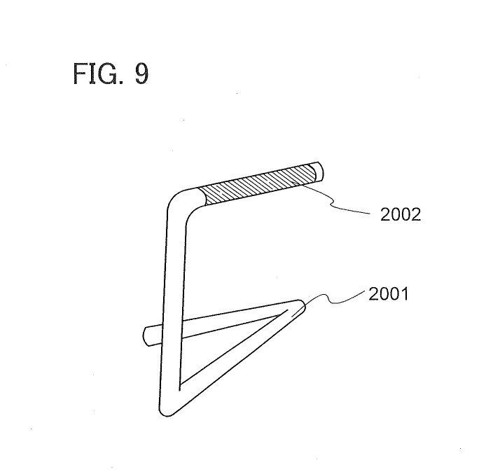

FIG. 9 illustrates an example of a desk lamp which is one embodiment of the present invention. The desk lamp illustrated in FIG. 9 includes a housing 2001 and a light source 2002; and a lighting device including a light-emitting element is used as the light source 2002.

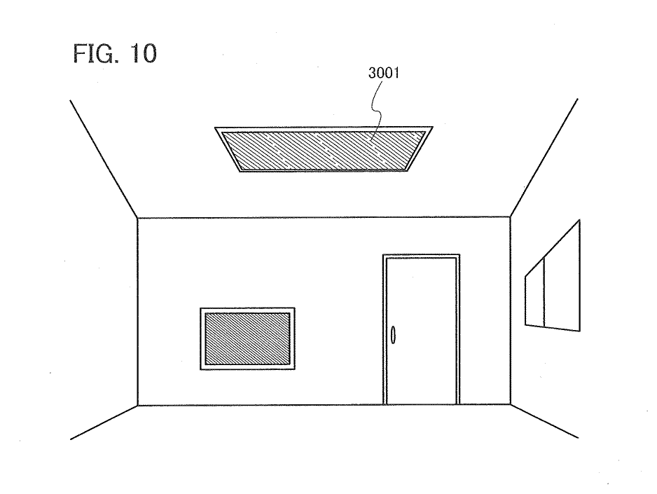

FIG. 10 illustrates an example of an indoor lighting device 3001. A light-emitting element including the organic compound of one embodiment of the present invention is preferably used in the lighting device 3001.

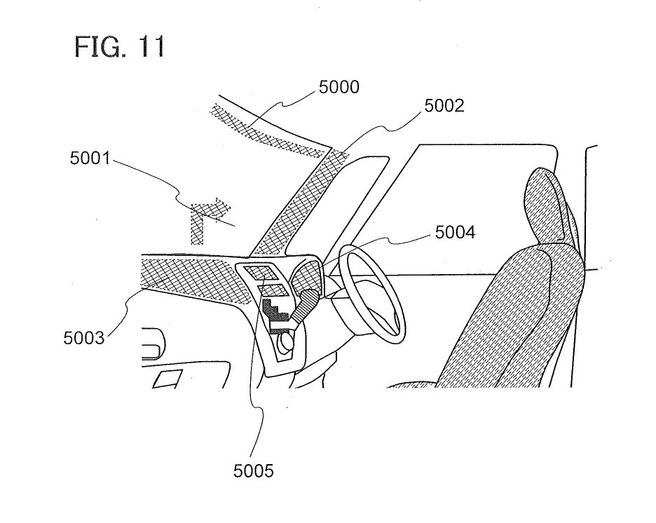

An automobile which is one embodiment of the present invention is illustrated in FIG. 11. In the automobile, light-emitting elements are used for a windshield and a dashboard. Display regions 5000 to 5005 are provided by using the light-emitting elements. The light-emitting elements preferably include the organic compound of one embodiment of the present invention, in which case the light-emitting elements can have low power consumption. This also suppresses power consumption of the display regions 5000 to 5005, showing suitability for use in an automobile.

The display regions 5000 and 5001 are display devices which are provided in the automobile windshield and which include the light-emitting elements. When a first electrode and a second electrode are formed of electrodes having light-transmitting properties in these light-emitting elements, what is called a see-through display device, through which the opposite side can be seen, can be obtained. Such a see-through display device can be provided even in the automobile windshield, without hindering the vision. Note that in the case where a transistor for driving or the like is provided, a transistor having a light-transmitting property, such as an organic transistor using an organic semiconductor material or a transistor using an oxide semiconductor, is preferably used.

The display region 5002 is a display device which is provided in a pillar portion and which includes the light-emitting element. The display region 5002 can compensate for the view hindered by the pillar portion by showing an image taken by an imaging unit provided in the car body. Similarly, a display region 5003 provided in the dashboard can compensate for the view hindered by the car body by showing an image taken by an imaging unit provided in the outside of the car body, which leads to elimination of blind areas and enhancement of safety. Showing an image so as to compensate for the area which a driver cannot see makes it possible for the driver to confirm safety easily and comfortably.

The display region 5004 and the display region 5005 can provide a variety of kinds of information such as navigation information, a speedometer, a tachometer, a mileage, a fuel meter, a gearshift indicator, and air-condition setting. The content or layout of the display can be changed freely by a user as appropriate. Note that such information can also be shown by the display regions 5000 to 5003. The display regions 5000 to 5005 can also be used as lighting devices.

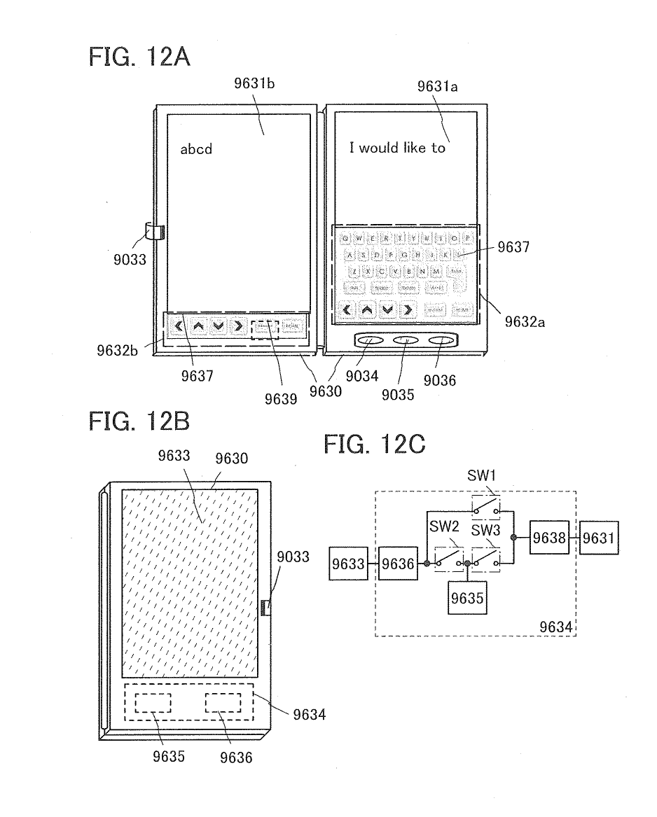

FIGS. 12A and 12B illustrate an example of a foldable tablet terminal. FIG. 12A illustrates the tablet terminal which is unfolded. The tablet terminal includes a housing 9630, a display portion 9631a, a display portion 9631b, a display mode switch 9034, a power switch 9035, a power-saving mode switch 9036, and a clip 9033. Note that in the tablet terminal, one or both of the display portion 9631a and the display portion 9631b is/are formed using a light-emitting device which includes the light-emitting element containing the organic compound of one embodiment of the present invention.

Part of the display portion 9631a can be a touchscreen region 9632a and data can be input when a displayed operation key 9637 is touched. Although half of the display portion 9631a has only a display function and the other half has a touchscreen function, one embodiment of the present invention is not limited to the structure. The whole display portion 9631a may have a touchscreen function. For example, a keyboard can be displayed on the entire region of the display portion 9631a so that the display portion 9631a is used as a touchscreen, and the display portion 9631b can be used as a display screen.

Like the display portion 9631a, part of the display portion 9631b can be a touchscreen region 9632b. When a switching button 9639 for showing/hiding a keyboard on the touchscreen is touched with a finger, a stylus, or the like, the keyboard can be displayed on the display portion 9631b.

Touch input can be performed in the touchscreen region 9632a and the touchscreen region 9632b at the same time.

The display mode switch 9034 can switch the display between portrait mode, landscape mode, and the like, and between monochrome display and color display, for example. The power-saving mode switch 9036 can control display luminance in accordance with the amount of external light in use of the tablet terminal sensed by an optical sensor incorporated in the tablet terminal. Another sensing device including a sensor such as a gyroscope or an acceleration sensor for sensing inclination may be incorporated in the tablet terminal, in addition to the optical sensor.

Although FIG. 12A illustrates an example in which the display portion 9631a and the display portion 9631b have the same display area, one embodiment of the present invention is not limited to the example. The display portion 9631a and the display portion 9631b may have different display areas and different display quality. For example, higher resolution images may be displayed on one of the display portions 9631a and 9631b.

FIG. 12B illustrates the tablet terminal which is folded. The tablet terminal in this embodiment includes the housing 9630, a solar cell 9633, a charge and discharge control circuit 9634, a battery 9635, and a DCDC converter 9636. In FIG. 12B, a structure including the battery 9635 and the DCDC converter 9636 is illustrated as an example of the charge and discharge control circuit 9634.

Since the tablet terminal is foldable, the housing 9630 can be closed when the tablet terminal is not in use. As a result, the display portion 9631a and the display portion 9631b can be protected, thereby providing a tablet terminal with high endurance and high reliability for long-term use.

The tablet terminal illustrated in FIGS. 12A and 12B can have other functions such as a function of displaying various kinds of data (e.g., a still image, a moving image, and a text image), a function of displaying a calendar, a date, the time, or the like on the display portion, a touch-input function of operating or editing the data displayed on the display portion by touch input, and a function of controlling processing by various kinds of software (programs).

The solar cell 9633 provided on a surface of the tablet terminal can supply power to the touchscreen, the display portion, a video signal processing portion, or the like. Note that a structure in which the solar cell 9633 is provided on one or both surfaces of the housing 9630 is preferable because the battery 9635 can be charged efficiently.

The structure and operation of the charge and discharge control circuit 9634 illustrated in FIG. 12B are described with reference to a block diagram of FIG. 12C. FIG. 12C illustrates the solar cell 9633, the battery 9635, the DCDC converter 9636, a converter 9638, switches SW1 to SW3, and a display portion 9631. The battery 9635, the DCDC converter 9636, the converter 9638, and the switches SW1 to SW3 correspond to the charge and discharge control circuit 9634 illustrated in FIG. 12B.

First, description is made on an example of the operation in the case where power is generated by the solar cell 9633 with the use of external light. The voltage of the power generated by the solar cell is raised or lowered by the DCDC converter 9636 so as to be voltage for charging the battery 9635. Then, when power from the solar cell 9633 is used for the operation of the display portion 9631, the switch SW1 is turned on and the voltage of the power is raised or lowered by the converter 9638 so as to be voltage needed for the display portion 9631. When images are not displayed on the display portion 9631, the switch SW1 is turned off and the switch SW2 is turned on so that the battery 9635 is charged.

Although the solar cell 9633 is described as an example of a power generation unit, the power generation unit is not particularly limited, and the battery 9635 may be charged by another power generation unit such as a piezoelectric element or a thermoelectric conversion element (Peltier element). The battery 9635 may be charged by a non-contact power transmission module capable of performing charging by transmitting and receiving power wirelessly (without contact), or another charge unit used in combination, and the power generation unit is not necessarily provided.

Note that the organic compound of one embodiment of the present invention can be used for an organic thin-film solar cell. Specifically, the organometallic complex can be used in a carrier-transport layer since the organometallic complex has a carrier-transport property. The organometallic complex can be photoexcited and hence can be used in a power generation layer.

One embodiment of the present invention is not limited to the tablet terminal having the shape illustrated in FIGS. 12A to 12C as long as the display portion 9631 is included.

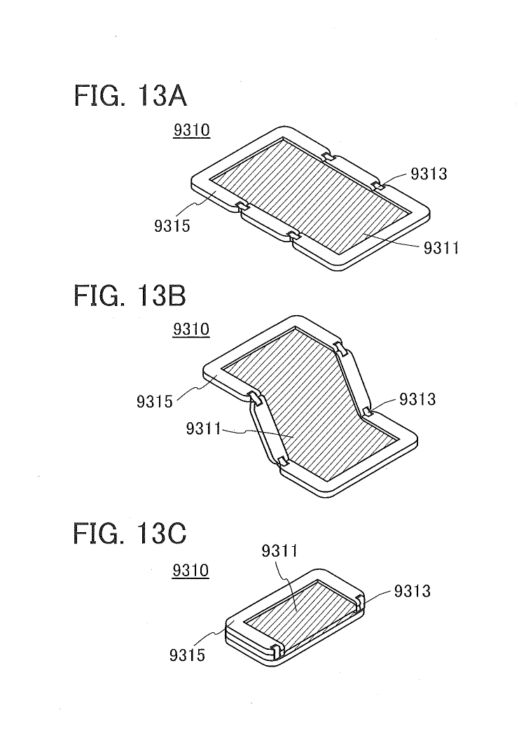

FIGS. 13A to 13C illustrate a foldable portable information terminal 9310. FIG. 13A illustrates the portable information terminal 9310 that is opened. FIG. 13B illustrates the portable information terminal 9310 that is being opened or being folded. FIG. 13C illustrates the portable information terminal 9310 that is folded. The portable information terminal 9310 is highly portable when folded. When the portable information terminal 9310 is opened, a seamless large display region is highly browsable.

A display panel 9311 is supported by three housings 9315 joined together by hinges 9313. Note that the display panel 9311 may be a touch panel (an input/output device) including a touch sensor (an input device). By bending the display panel 9311 at a connection portion between two housings 9315 with the use of the hinges 9313, the portable information terminal 9310 can be reversibly changed in shape from an opened state to a folded state. The light-emitting device of one embodiment of the present invention can be used for the display panel 9311. A display region 9312 in the display panel 9311 is a display region that is positioned at the side surface of the portable information terminal 9310 that is folded. On the display region 9312, information icons, file shortcuts of frequently used applications or programs, and the like can be displayed, and confirmation of information and start of application can be smoothly performed.

### Example 1

### Synthesis Example 1

In Synthesis Example 1, synthesis of 11-phenyl-5-[4-(10-phenylanthracen-9-yl)phenyl]-11H-benzo[a]carbazole (abbreviation: PaBCPA) that is an organic compound of one embodiment of the present invention and represented by Structural Formula (100) shown below is described in detail. The structural formula of PaBCPA is shown below.

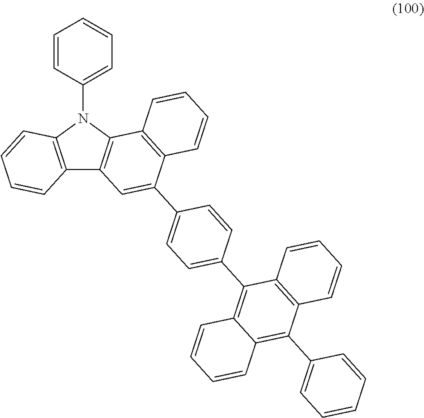 [SMILES: c1ccc(-c2c3ccccc3c(-c3ccc(-c4cc5c6ccccc6n(-c6ccccc6)c5c5ccccc45)cc3)c3ccccc23)cc1]

### Step 1: Synthesis of 11-phenyl-11H-benzo[a]carbazole

Into a 200-mL three-neck flask were placed 1.2 g (5.5 mmol) of 11H-benzo[a]carbazole, 0.86 g (5.5 mol) of bromobenzene, and 1.0 g (11 mmol) of sodium tert-butoxide. After the atmosphere in the flask was replaced with nitrogen, 28 mL of toluene and 2.8 mL of tri-(tert-butyl)phosphine (a 10 wt % hexane solution) was added to this mixture. This mixture was degassed by being stirred while the pressure was reduced. After the degassing, 0.16 g (0.28 mmol) of bis(dibenzylideneacetone)palladium(0) was added to this mixture. This mixture was stirred at 110° C. for 6 hours under a nitrogen stream. After the stirring, this mixture was cooled to room temperature, and the mixture was subjected to suction filtration to remove a solid. An oily substance obtained by concentration of the obtained filtrate was suction-filtered through Celite (produced by Wako Pure Chemical Industries, Ltd., Catalog No. 531-16855), Florisil (produced by Wako Pure Chemical Industries, Ltd., Catalog No. 540-00135), and alumina. An oily substance obtained by concentration of the obtained filtrate was dried under reduced pressure, so that 1.6 g of a pale brown oily substance of the target substance was obtained in a yield of 99%. A synthesis scheme of Step 1 is shown below.

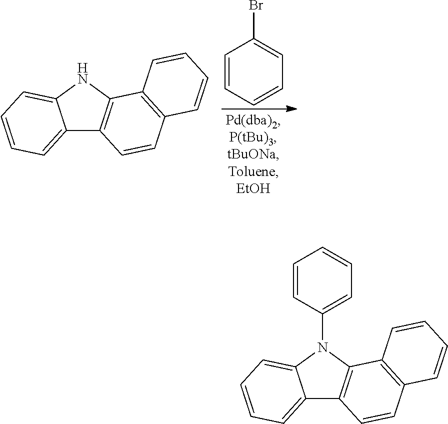

### Step 2: Synthesis of 5-bromo-11-phenyl-11H-benzo[a]carbazole

Into a 100-mL Mayer flask were put 1.6 g (5.5 mmol) of 11-phenyl-11H-benzo[a]carbazole, 30 mL of chloroform, and 0.98 g (5.5 mmol) of N-bromosuccinimide, and the mixture was stirred at room temperature for 18 hours. After the stirring, approximately 20 mL of water was added to this solution, and stirring was performed for 4 hours. After the stirring, an aqueous layer of this mixture was subjected to extraction with chloroform, and a solution of the extract and the organic layer were combined and washed with water. After that, the organic layer was dried with magnesium sulfate. After the drying, this mixture was gravity-filtered, and then the obtained filtrate was concentrated to give an oily substance. The obtained oily substance was recrystallized with toluene/hexane, whereby 1.2 g of a white powder that was a target substance was obtained in a yield of 57%. A synthesis scheme of Step 2 is shown below.

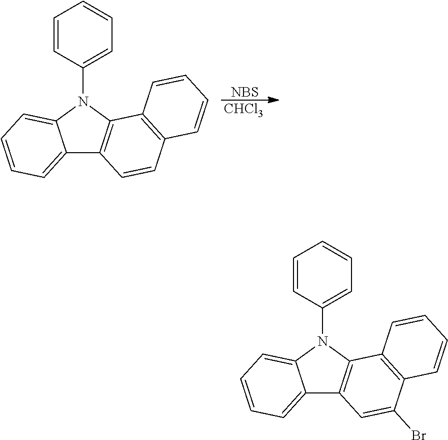

### Step 3: Synthesis of 11-phenyl-11H-benzo[a]carbazole-5-boronic acid

Into a 50-mL three-neck flask was put 1.2 g (3.2 mmol) of 5-bromo-11-phenyl-11H-benzo[a]carbazole, and the atmosphere in the flask was replaced with nitrogen. Into the flask, 16 mL of tetrahydrofuran (THF) was added, and this mixture was cooled down to −80° C. Then, 2.3 mL (3.6 mmol) of n-butyllithium (a 1.6 mol/L hexane solution) was dripped into this mixture with a syringe. After the dripping, this mixture was stirred at the same temperature for 2 hours. After the stirring, 0.80 mL (7.2 mmol) of trimethyl borate was added to this mixture, and the mixture was stirred for 15 hours while the temperature was returned to room temperature. After the stirring, approximately 10 mL of dilute hydrochloric acid (1.0 mol/L) was added to this mixture, and the mixture was stirred for 1 hour. After the stirring, the aqueous layer of this mixture was subjected to extraction with ethyl acetate, and a solution of the extract and the organic layer were combined and washed with a saturated aqueous solution of sodium hydrogen carbonate and saturated brine. The organic layer was dried with magnesium sulfate. After the drying, the mixture was gravity-filtered, and the obtained filtrate was concentrated to give a brown oily substance. Chloroform/hexane was added to the obtained solid, the mixture was irradiated with ultrasonic waves, and a solid was collected by suction filtration, whereby 0.36 g of a white powder that was a target substance was obtained in a yield of 33%. A synthesis scheme of Step 3 is shown below.

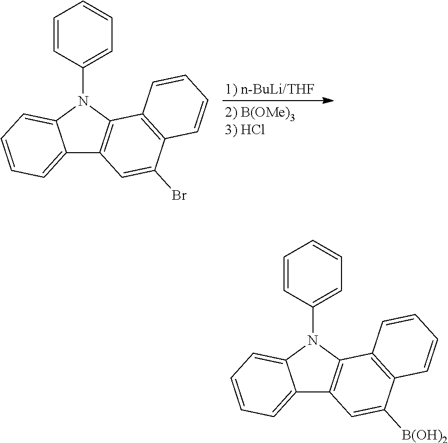 [SMILES: Brc1cc2c3ccccc3n(-c3ccccc3)c2c2ccccc12 ; OB(O)c1cc2c3ccccc3n(-c3ccccc3)c2c2ccccc12]

### Step 4: Synthesis of 11-phenyl-5-[4-(10-phenylanthracen-9-yl)phenyl]-11H-benzo[a]carbazole (abbreviation: PaBCPA)

Into a 50-mL three-neck flask were put 1.0 g (2.5 mmol) of 9-(4-bromophenyl)-10-phenylanthracene, 0.87 g (2.5 mmol) of 11-phenyl-11H-benzo[a]carbazole-5-boronic acid, and 0.18 g (0.60 mmol) of tri(ortho-tolyl)phosphine, and the atmosphere in the flask was replaced with nitrogen. To this mixture were added 10 mL of toluene, 3.0 mL of ethanol, and 3.0 mL of an aqueous solution of potassium carbonate (2.0 mol/L). While the pressure was reduced, this mixture was stirred to be degassed. To this mixture was added 28 mg (0.12 mmol) of palladium(II) acetate, and the mixture was stirred under a nitrogen stream at 80° C. for 6 hours. After the stirring, an aqueous layer of the obtained mixture was subjected to extraction with toluene, and a solution of the extract and the organic layer were combined and washed with saturated brine. The organic layer was dried with magnesium sulfate, and this mixture was gravity-filtered. The obtained filtrate was concentrated to give an oily substance, and the oily substance was purified by silica gel column chromatography (developing solvent, hexane:toluene=7:1) to give a solid. The obtained solid was recrystallized with toluene/hexane, so that 0.82 g of a white solid that was a target substance was obtained in a yield of 52%. A synthesis scheme of Step 4 is shown below.

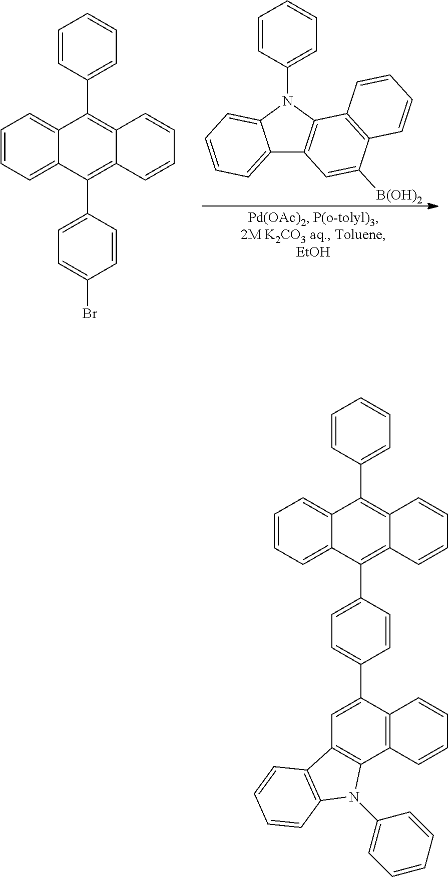

By a train sublimation method, 0.82 g of the obtained white solid was purified. In the purification by sublimation, PaBCPA was heated at 290° C. under a pressure of 3.2 Pa with a flow rate of argon gas of 5.0 mL/min. After the purification by sublimation, 0.67 g of a white solid of PaBCPA was obtained at a collection rate of 82%.

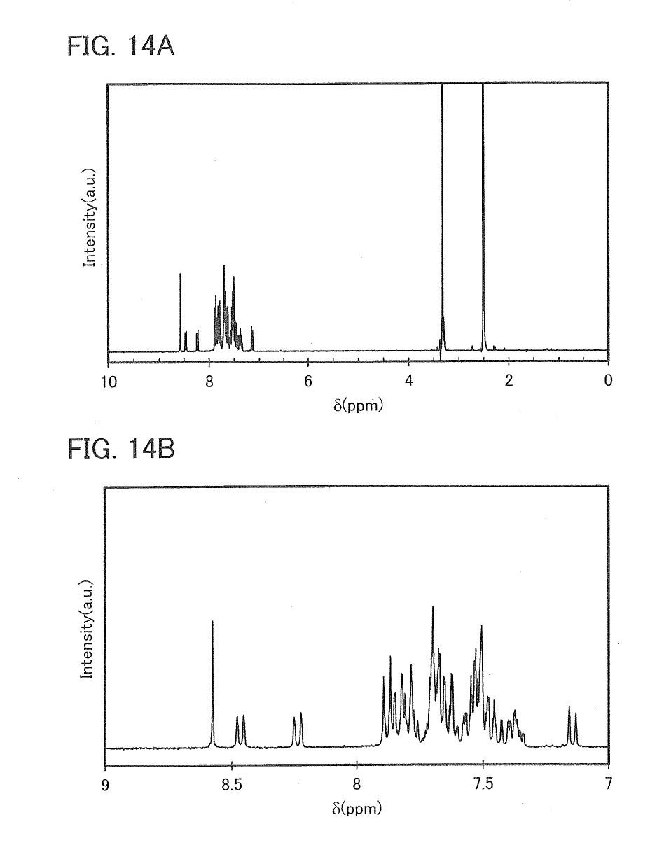

¹H NMR data of the obtained white solid are shown below. FIGS. 14A and 14B are ¹H-NMR charts. Note that FIG. 14B is a chart showing an enlarged part of FIG. 14A in the range of 7.00 ppm to 9.00 ppm. These results indicate that PaBCPA, which is the organic compound of one embodiment of the present invention, was obtained.

¹H NMR (DMSO-d⁶, 300 MHz): δ=7.15 (d, J₁=8.4 Hz, 1H), 7.34-7.90 (m, 27H), 8.24 (d, J₁=8.4 Hz, 1H), 8.47 (d, J₁=7.5 Hz, 1H), 8.58 (s, 1H)

The thermogravimetry-differential thermal analysis (TG/DTA) of PaBCPA was performed. The measurement was performed using a high vacuum differential type differential thermal balance (TG-DTA2410SA, manufactured by Bruker AXS K.K.). The measurement was carried out under a nitrogen stream (flow rate: 200 mL/min) at normal pressure at a temperature rising rate of 10° C./min. From relationship between weight and temperature (thermogravimetry), the 5% weight loss temperature and the melting point of PaBCPA were 452° C. and 322° C., respectively. This indicates that PaBCPA has high heat resistance.

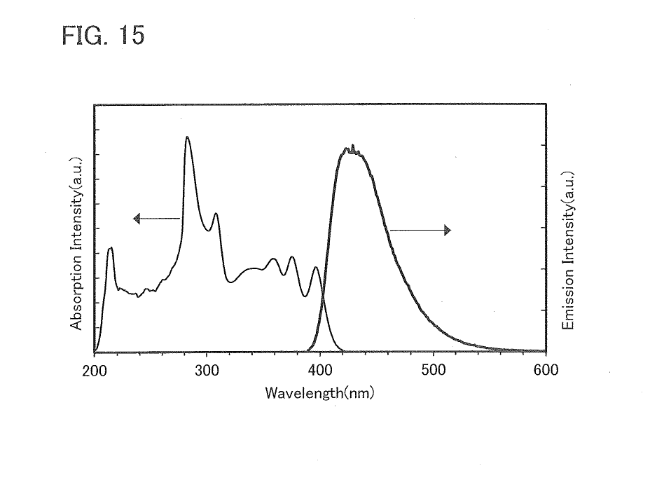

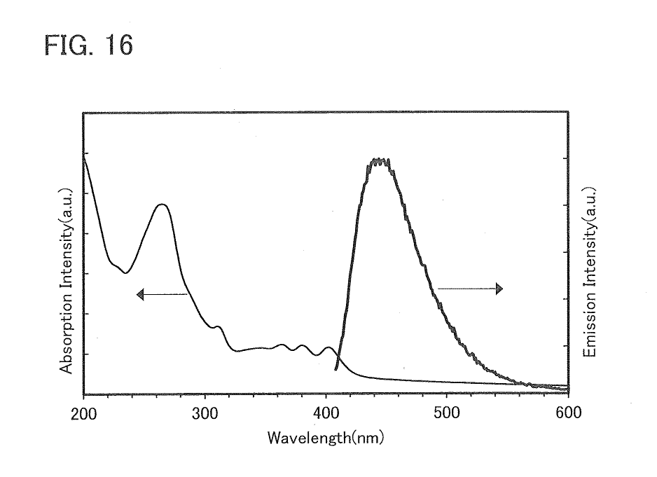

Next, the ultraviolet-visible absorption spectra (hereinafter, simply referred to as “absorption spectrum”) and emission spectra of a toluene solution and a solid thin film of PaBCPA were measured. The solid thin film was formed over a quartz substrate by a vacuum evaporation method. The absorption spectra were measured using an ultraviolet-visible light spectrophotometer (V550 type manufactured by JASCO Corporation). The emission spectra were measured using a fluorescence spectrophotometer (FS920 manufactured by Hamamatsu Photonics K.K.). FIG. 15 shows the absorption spectrum and the emission spectrum of the toluene solution. FIG. 16 shows the absorption spectrum and the emission spectrum of the solid thin film.

According to FIG. 15, absorption peaks of the toluene solution of PaBCPA are observed at around 282 nm, 308 nm, 343 nm, 359 nm, 375 nm, and 396 nm, and an emission wavelength peak is observed at 424 nm (excitation wavelength: 376 nm).

According to FIG. 16, absorption peaks of the thin film of PaBCPA are observed at around 224 nm, 265 nm, 285 nm, 311 nm, 338 nm, 363 nm, 380 nm, and 402 nm, and an emission wavelength peak is observed at 447 nm (excitation wavelength: 402 nm).

The HOMO level and the LUMO level of PaBCPA were obtained through a cyclic voltammetry (CV) measurement. A calculation method is described below.

An electrochemical analyzer (ALS model 600A or 600C, manufactured by BAS Inc.) was used as a measurement apparatus. As for a solution used for the CV measurement, dehydrated dimethylformamide (DMF, product of Sigma-Aldrich Inc., 99.8%, catalog No. 22705-6) was used as a solvent, and tetra-n-butylammonium perchlorate (n-Bu₄NClO₄, product of Tokyo Chemical Industry Co., Ltd., catalog No. T0836), which was a supporting electrolyte, was dissolved in the solvent such that the concentration of tetra-n-butylammonium perchlorate was 100 mmol/L. Further, the object to be measured was also dissolved in the solvent such that the concentration thereof was 2 mmol/L. A platinum electrode (PTE platinum electrode, manufactured by BAS Inc.) was used as a working electrode, another platinum electrode (Pt counter electrode for VC-3 (5 cm), manufactured by BAS Inc.) was used as an auxiliary electrode, and an Ag/Ag⁺ electrode (RE7 reference electrode for nonaqueous solvent, manufactured by BAS Inc.) was used as a reference electrode. Note that the measurement was performed at room temperature (20° C. to 25° C.). In addition, the scan speed at the CV measurement was set to 0.1 V/sec, and an oxidation potential Ea [V] and a reduction potential Ec [V] with respect to the reference electrode were measured. Furthermore, the CV measurement was repeated 100 times, and the oxidation-reduction wave at the hundredth cycle and the oxidation-reduction wave at the first cycle were compared with each other to examine the electrochemical stability of the compound. Note that Ea represents an intermediate potential of an oxidation-reduction wave, and Ec represents an intermediate potential of a reduction-oxidation wave. Here, the potential energy of the reference electrode used in this example with respect to the vacuum level is found to be −4.94 [eV], and thus, the HOMO level and the LUMO level can be obtained from the following formula: HOMO level [eV]=−4.94−Ea and LUMO level [eV]=−4.94−Ec.

As a result, it was found that the HOMO level of PaBCPA was −5.68 eV and the LUMO level thereof was −2.71 eV. After the hundredth cycle, the peak intensity of the oxidation wave maintained 95% of that of the oxidation wave at the first cycle, and the peak intensity of the reduction wave maintained 85% of that of the reduction wave at the first cycle, which indicates that PaBCPA is a compound resistant to oxidation and reduction.

### Example 2

### Synthesis Example 2

In Synthesis Example 2, a method for synthesizing 5-phenyl-11-[4-(10-phenylanthracen-9-yl)phenyl]-11H-benzo[a]carbazole (abbreviation: aBCzPAP) that is an organic compound of one embodiment of the present invention and represented by Structural Formula (136) is described. Structural Formula of aBCzPAP is shown below.

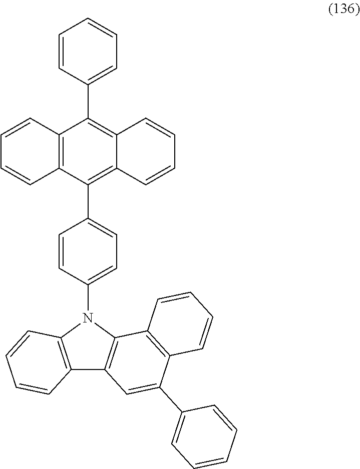 [SMILES: c1ccc(-c2c3ccccc3c(-c3ccc(-n4c5ccccc5c5cc(-c6ccccc6)c6ccccc6c54)cc3)c3ccccc23)cc1]

### Step 1: Synthesis of 5-bromo-11H-benzo[a]carbazole

Into a 200-mL Mayer flask were put 1.0 g (4.6 mmol) of 11H-benzo[a]carbazole, 40 mL of chloroform, and 0.81 g (4.6 mmol) of N-bromosuccinimide, and the mixture was stirred at room temperature for 16 hours. After the stirring, approximately 40 mL of water was added to this solution and stirring was performed for 2 hours. After the stirring, this mixture was washed with water, and the organic layer was dried with magnesium sulfate. After the drying, this mixture was gravity-filtered, and the filtrate was concentrated to give a solid. The solid was recrystallized with chloroform/hexane, whereby 1.0 g of a white powder that was a target substance was obtained in a yield of 75%. A synthesis scheme of Step 1 is shown below.

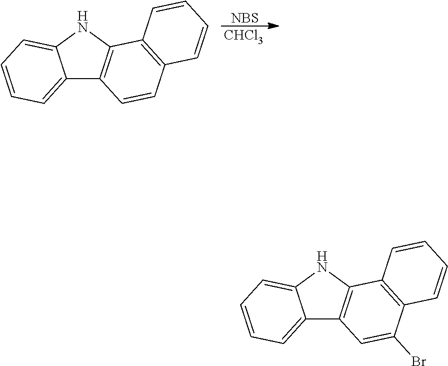

### Step 2: Synthesis of 5-phenyl-11H-benzo[a]carbazole

Into a 50-mL three-neck flask were put 0.95 g (3.2 mmol) of 5-bromo-11H-benzo[a]carbazole, 0.39 g (3.2 mmol) of phenylboronic acid, and 0.24 g (0.80 mmol) of tri(ortho-tolyl)phosphine, and the atmosphere in the flask was replaced with nitrogen. To this mixture were added 10 mL of toluene, 6.0 mL of ethanol, and 4.0 mL of an aqueous solution of potassium carbonate (2.0 mol/L). While the pressure was reduced, this mixture was stirred to be degassed. To this mixture was added 36 mg (0.12 mmol) of palladium(II) acetate, and the mixture was stirred under a nitrogen stream at 80° C. for 3 hours. After the stirring, an aqueous layer of the obtained mixture was subjected to extraction with toluene, and a solution of the extract and the organic layer were combined and washed with saturated brine. The organic layer was dried with magnesium sulfate, and this mixture was gravity-filtered. An oily substance obtained by concentration of the obtained filtrate was suction-filtered through Celite (produced by Wako Pure Chemical Industries, Ltd., Catalog No. 531-16855), Florisil (produced by Wako Pure Chemical Industries, Ltd., Catalog No. 540-00135), and alumina. An oily substance obtained by concentration of the obtained filtrate was recrystallized with toluene/hexane, so that 0.60 g of a white powder of the target substance was obtained in a yield of 67%. A synthesis scheme of Step 2 is shown below.

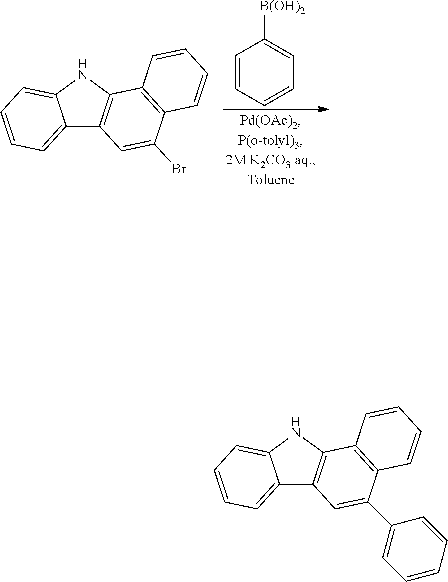

### Step 3: Synthesis of 5-phenyl-11-[4-(10-phenylanthracen-9-yl)phenyl]-11H-benzo[a]carbazole (abbreviation: aBCzPAP)

In a 50-mL three-neck flask were put 0.83 g (2.0 mmol) of 9-(4-bromophenyl)-10-phenylanthracene, 0.60 g (2.0 mmol) of 5-phenyl-11H-benzo[a]carbazole, and 0.38 g (4.0 mmol) of sodium tert-butoxide. After the atmosphere in the flask was replaced with nitrogen, 10 mL of toluene and 1.0 mL of tri-(tert-butyl)phosphine (a 10 wt % hexane solution) was added to this mixture. This mixture was degassed by being stirred while the pressure was reduced. After the degassing, 57 mg (0.10 mmol) of bis(dibenzylideneacetone)palladium(0) was added to this mixture. This mixture was stirred at 110° C. for 5 hours under a nitrogen stream. After the stirring, this mixture was subjected to suction filtration to give a filtrate. An oily substance obtained by concentration of the obtained filtrate was purified by silica gel column chromatography (developing solvent, hexane:toluene=7:1) to give an oily substance. The obtained oily substance was recrystallized with toluene/hexane, so that 1.0 g of a white powder of the target substance was obtained in a yield of 80%. A synthesis scheme of Step 3 is shown below.

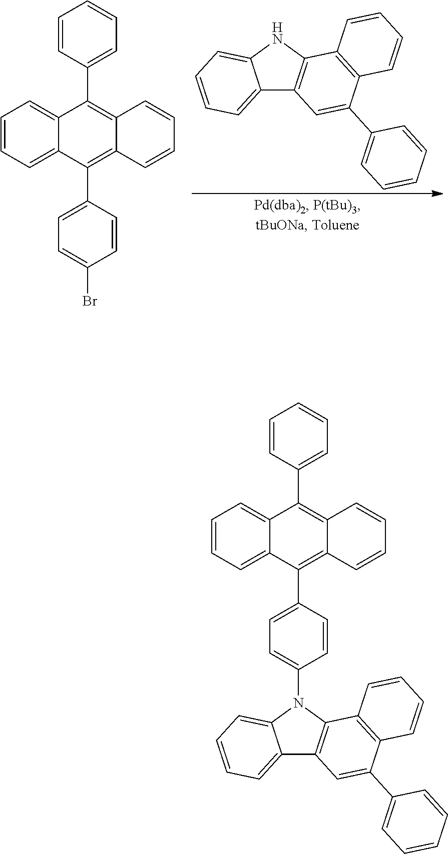

By a train sublimation method, 1.0 g of the obtained white powder was purified. In the purification by sublimation, the white powder was heated at 270° C. under a pressure of 3.2 Pa with a flow rate of argon gas of 5.0 mL/min. After the purification by sublimation, 0.80 g of a white solid of aBCzPAP was obtained at a collection rate of 80%.

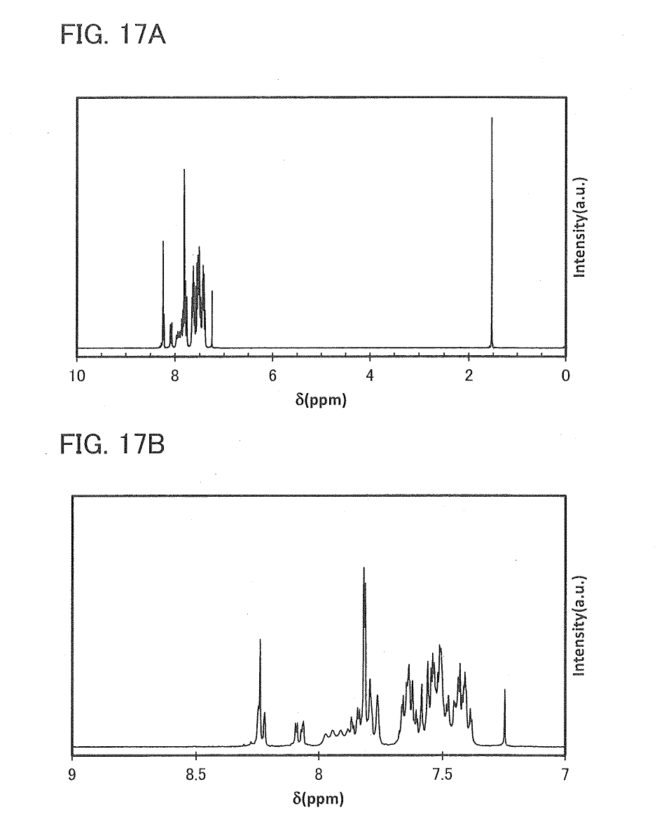

¹H NMR data of the obtained white solid are shown below. FIGS. 17A and 17B are ¹H-NMR charts. Note that FIG. 17B is a chart showing an enlarged part of FIG. 17A in the range of 7.00 ppm to 9.00 ppm. These results indicate that aBCzPAP, which is the organic compound of one embodiment of the present invention, was obtained.

¹H NMR (CDCl₃, 300 MHz): δ=7.39-7.67 (m, 19H), 7.76-7.94 (min, 9H), 7.96 (dd, J₁=6.9 Hz, J₂=1.8 Hz, 1H), 8.22-8.25 (m, 2H).

TG-DTA analysis was performed on aBCzPAP in a manner similar to that for PaBCPA. From relationship between weight and temperature (thermogravimetry), the 5% weight loss temperature and the melting point of aBCzPAP were 439° C. and 261° C., respectively. This indicates that aBCzPAP has high heat resistance.

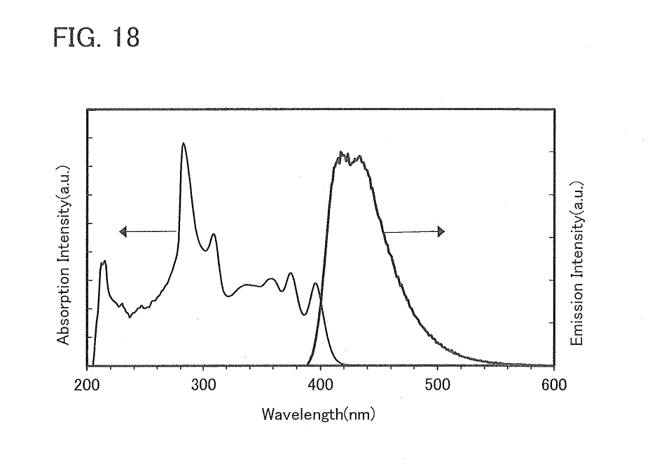

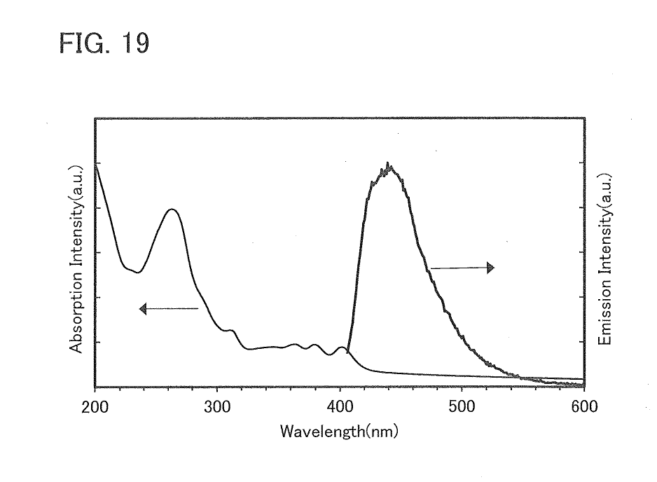

Next, the ultraviolet-visible absorption spectra (hereinafter, simply referred to as “absorption spectrum”) and emission spectra of a toluene solution and a solid thin film of aBCzPAP were measured in a manner similar to that for PaBCPA. FIG. 18 shows the absorption spectrum and the emission spectrum of the toluene solution. FIG. 19 shows the absorption spectrum and the emission spectrum of the solid thin film.

According to FIG. 18, absorption peaks of the toluene solution of aBCzPAP are observed at around 282 nm, 309 nm, 337 nm, 358 nm, 375 nm, and 396 nm, and emission wavelength peaks are observed at 417 nm and 431 nm (excitation wavelength: 376 nm).

According to FIG. 19, absorption peaks of the thin film of aBCzPAP are observed at around 263 nm, 311 nm, 346 nm, 363 nm, 379 nm, and 401 nm, and an emission wavelength peak is observed at 440 nm (excitation wavelength: 400 nm).

The HOMO level and the LUMO level of aBCzPAP were obtained through a cyclic voltammetry (CV) measurement. The calculation method was the same as that described in Synthesis Example 1. In addition, the hundred-cycle measurement was performed as in Synthesis Example 1.

As a result, it was found that the HOMO level of aBCzPAP was −5.71 eV and the LUMO level thereof was −2.74 eV. After the hundredth cycle, the peak intensity of the oxidation wave maintained 92% of that of the oxidation wave at the first cycle, and the peak intensity of the reduction wave maintained 76% of that of the reduction wave at the first cycle, which indicates that aBCzPAP is a compound resistant to oxidation and reduction.

### Example 3

### Synthesis Example 3

In Synthesis Example 3, synthesis of 5-[4-(9-phenanthryl)phenyl]-11-phenyl-11H-benzo[a]carbazole (abbreviation: PaBCPPn) that is an organic compound of one embodiment of the present invention and represented by Structural Formula (126) shown below is described in detail. The structural formula of PaBCPPn is shown below.

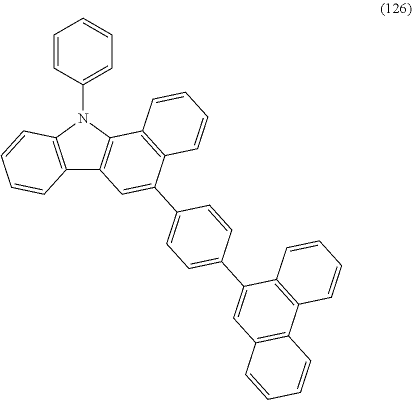 [SMILES: c1ccc(-n2c3ccccc3c3cc(-c4ccc(-c5cc6ccccc6c6ccccc56)cc4)c4ccccc4c32)cc1]

### Step 1: Synthesis of 11-phenyl-11H-benzo[a]carbazole

This step is similar to Step 1 in Synthesis Example 1.

### Step 2: Synthesis of 5-bromo-11-phenyl-11H-benzo[a]carbazole

This step is similar to Step 2 in Synthesis Example 1.

### Step 3: Synthesis of 5-[4-(9-phenanthryl)phenyl]-11-phenyl-11H-benzo[a]carbazole (abbreviation: PaBCPPn)

Into a 200-mL three-neck flask were put 1.5 g (4.0 mmol) of 5-bromo-11-phenyl-11H-benzo[a]carbazole, 1.2 g (4.0 mmol) of 4-(9-phenanthryl)phenylboronic acid, 0.19 g (0.60 mmol) of tris(2-methylphenyl)phosphine, 15 mL of toluene, 5 mL of ethanol, and 6 mL of an aqueous solution of potassium carbonate (2 mol/L). This mixture was degassed under reduced pressure, and a nitrogen gas was made to flow continuously in the system. This mixture was heated to 60° C., 48 mg (0.20 mmol) of palladium(II) acetate was added thereto, and this mixture was stirred at 80° C. for 2.5 hours. After the stirring, an aqueous layer of the obtained mixture was subjected to extraction with toluene. A solution of the extract and the organic layer were combined, washed with saturated brine, and dried with anhydrous magnesium sulfate. The obtained mixture was gravity-filtered, and then the obtained filtrate was concentrated to give a brown oily substance. The obtained oily substance was purified by high performance liquid chromatography (developing solvent: chloroform) and recrystallized with ethanol/hexane, whereby 1.5 g of a white solid that was a target substance was obtained in a yield of 72%. A synthesis scheme of Step 3 is shown below.

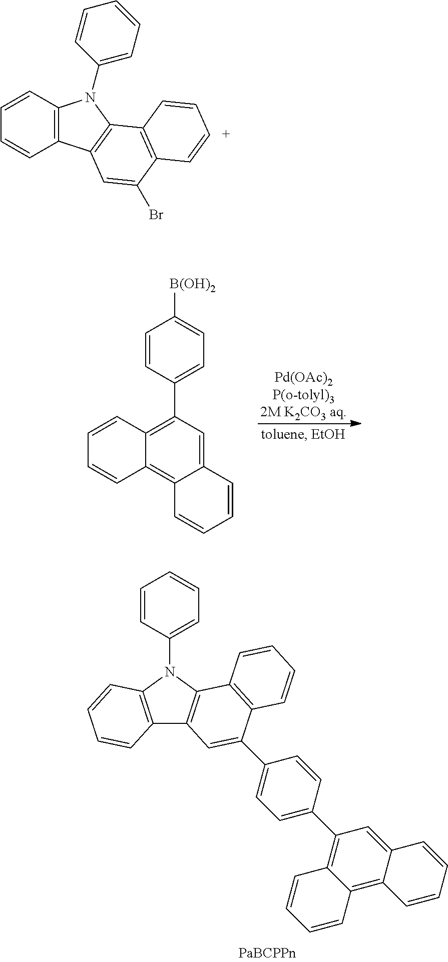 [SMILES: Brc1cc2c3ccccc3n(-c3ccccc3)c2c2ccccc12 ; OB(O)c1ccc(-c2cc3ccccc3c3ccccc23)cc1 ; c1ccc(-n2c3ccccc3c3cc(-c4ccc(-c5cc6ccccc6c6ccccc56)cc4)c4ccccc4c32)cc1]

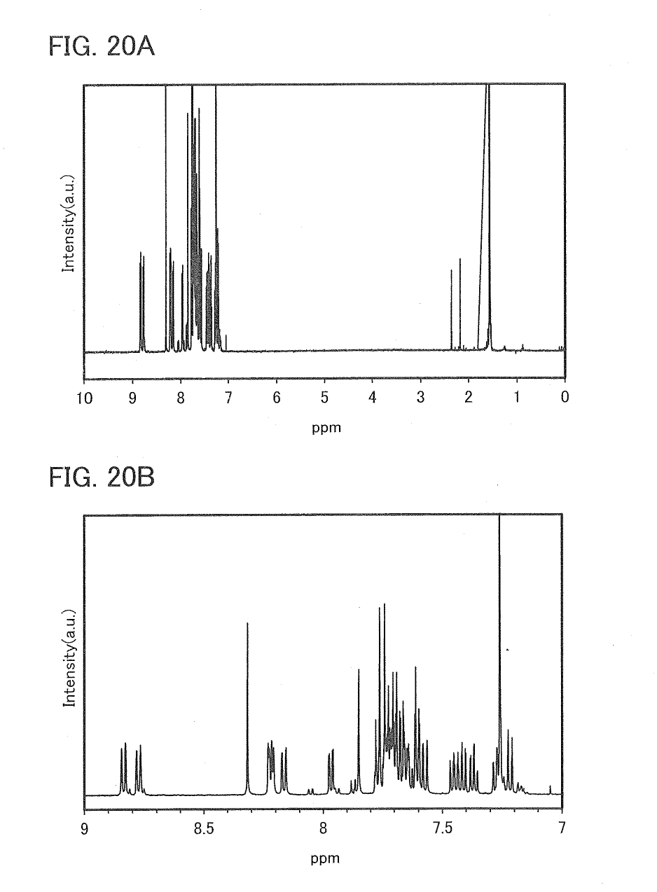

¹H NMR data of the obtained white solid are shown below. FIGS. 20A and 20B are ¹H-NMR charts. Note that FIG. 20B is a chart showing an enlarged part of FIG. 20A in the range of 7.00 ppm to 9.00 ppm. These results indicate that PaBCPPn, which is the organic compound of one embodiment of the present invention, was obtained.

¹H NMR (chloroform-d, 500 MHz): δ=7.22 (d, J=8.0 Hz, 1H), 7.27-7.19 (m, 1H), 7.37 (t, J=8.0 Hz, 1H), 7.40-7.47 (m, 2H), 7.57 (d, J=8.0 Hz, 1H), 7.60-7.74 (m, 11H), 7.77 (d, J=8.0 Hz, 2H), 7.85 (s, 1H), 7.97 (d, J=8.0 Hz, 1H), 8.16 (d, J=8.0 Hz, 1H), 8.21-8.23 (m, 2H), 8.32 (s, 1H), 8.78 (d, J=8.0 Hz, 1H), 8.84 (d, J=8.0 Hz, 1H)

By a train sublimation method, 1.5 g of the obtained solid was purified. In the purification by sublimation, the solid was heated at 280° C. for 15 hours under a pressure of 3.7 Pa with a flow rate of argon gas of 15 mL/min. After the purification, 1.1 g of a white solid of the target substance was obtained at a collection rate of 69%.

TG-DTA analysis was performed on PaBCPPn in a manner similar to that for PaBCPA. From relationship between weight and temperature (thermogravimetry), the 5% weight loss temperature of PaBCPPn was 402° C. This indicates that PaBCPPn has high heat resistance. Furthermore, differential scanning calorimetry (DSC measurement) of PaBCPPn was performed by Pyris1DSC manufactured by PerkinElmer, Inc. In the differential scanning calorimetry, after the temperature was raised from −10° C. to 300° C. at a temperature increase rate of 40° C./min, the temperature was held for a minute and then cooled to −10° C. at a temperature reduction rate of 40° C./min. This operation was repeated twice successively. It was found from the DSC measurement result of the second cycle that the glass transition point of PaBCPPn is 130° C., that is, PaBCPPn is a compound having high heat resistance.

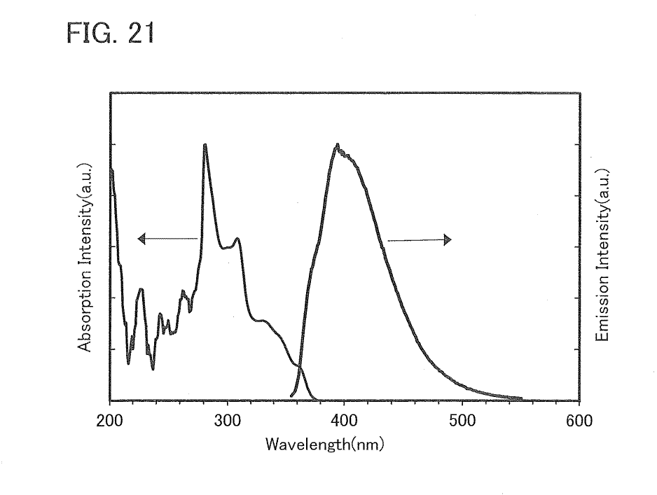

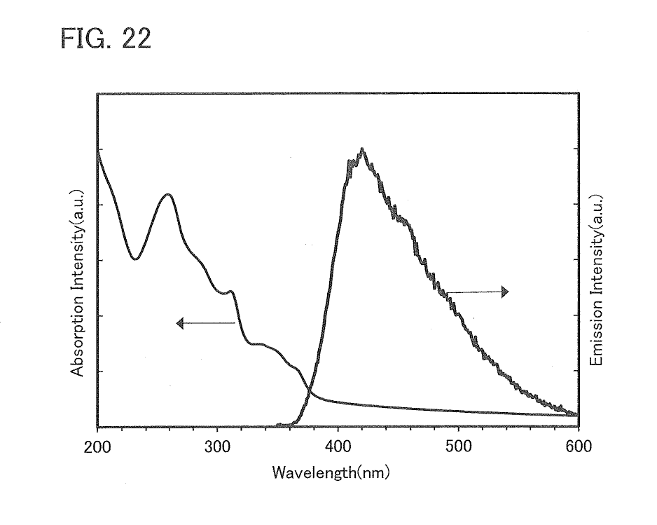

Next, the ultraviolet-visible absorption spectra (hereinafter, simply referred to as “absorption spectrum”) and emission spectra of a toluene solution and a solid thin film of PaBCPPn were measured in a manner similar to that for PaBCPA. FIG. 21 shows the absorption spectrum and the emission spectrum of the toluene solution. FIG. 22 shows the absorption spectrum and the emission spectrum of the solid thin film.

According to FIG. 21, absorption peaks of the toluene solution of PaBCPPn are observed at around 308 nm, 331 nm, and 344 nm, and an emission wavelength peak is observed at 390 nm (excitation wavelength: 350 nm).

According to FIG. 22, absorption peaks of the thin film of PaBCPPn are observed at around 259 nm, 289 nm, 311 nm, 336 nm, 349 nm, and 366 nm, and an emission wavelength peak is observed at 421 nm (excitation wavelength: 340 nm).

The HOMO level and the LUMO level of PaBCPPn were obtained through a cyclic voltammetry (CV) measurement. The calculation method was the same as that described in Synthesis Example 1. In addition, the hundred-cycle measurement was performed as in Synthesis Example 1.

As a result, it was found that the HOMO level of PaBCPPn was −5.72 eV and the LUMO level thereof was −2.28 eV. After the hundredth cycle, the peak intensity of the oxidation wave maintained 97% of that of the oxidation wave at the first cycle, which indicates that PaBCPPn is a compound highly resistant to oxidation.

### Example 4

In this example, a light-emitting element 1 which is one embodiment of the present invention and described in Embodiment is described in detail. Structural formulae of organic compounds used for the light-emitting element 1 are shown below.

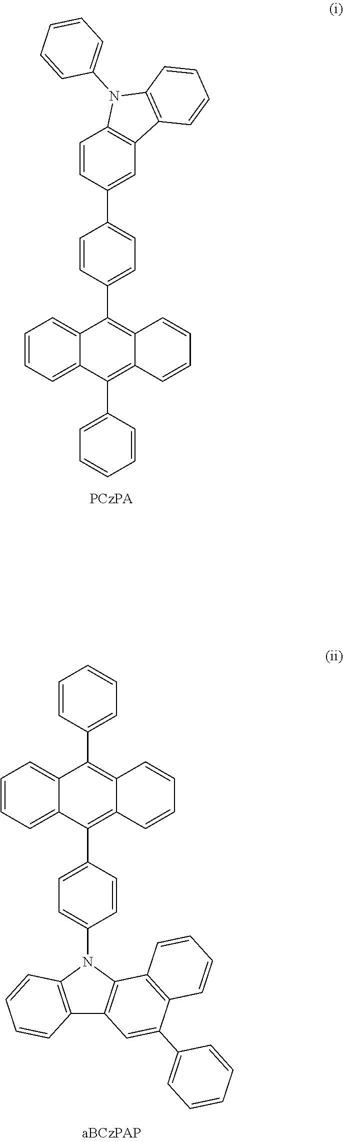 [SMILES: c1ccc(-c2c3ccccc3c(-c3ccc(-c4ccc5c(c4)c4ccccc4n5-c4ccccc4)cc3)c3ccccc23)cc1 ; c1ccc(-c2c3ccccc3c(-c3ccc(-n4c5ccccc5c5cc(-c6ccccc6)c6ccccc6c54)cc3)c3ccccc23)cc1]

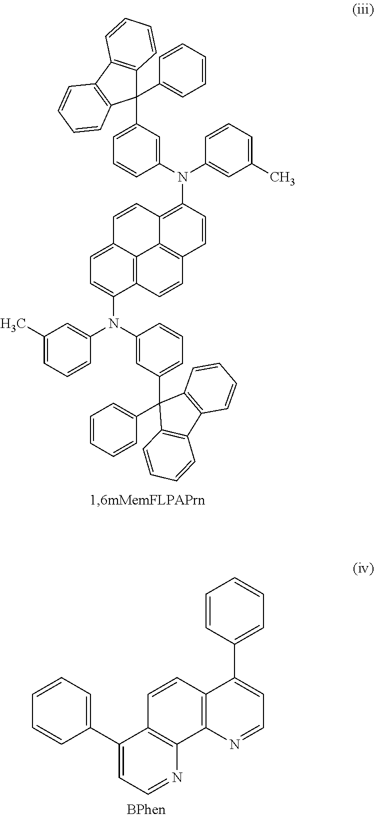

### (Manufacturing Method of Light-Emitting Element 1)

First, indium tin oxide containing silicon oxide (ITSO) was deposited over a glass substrate by a sputtering method, whereby the first electrode 101 was formed. The thickness of the first electrode 101 was set to 70 nm and the area of the electrode was set to 2 mm×2 nm.

Next, in pretreatment for forming the light-emitting element over the substrate, a surface of the substrate was washed with water and baked at 200° C. for 1 hour, and then UV ozone treatment was performed for 370 seconds.

Then, the substrate was transferred into a vacuum evaporation apparatus in which the pressure was reduced to approximately 10⁻⁴ Pa, and vacuum baking at 170° C. for 30 minutes was conducted in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus such that the surface on which the first electrode 101 was formed faced downward. After that, over the first electrode 101, 9-phenyl-3-[4-(10-phenyl-9-anthryl)phenyl]-9H-carabzole (abbreviation: PCzPA) represented by Structural Formula (i) shown above and molybdenum(VI) oxide were co-evaporated by an evaporation method using resistance heating, whereby the hole-injection layer 111 was formed. The hole-injection layer 111 was formed to have a thickness of 10 nm such that the weight ratio of PCzPA to molybdenum oxide was 4:2.

Next, PCzPA represented by the above structural formula was deposited to a thickness of 30 nm over the hole-injection layer 111 to form the hole-transport layer 112.

Furthermore, over the hole-transport layer 112, the light-emitting layer 113 was formed by co-evaporation of 5-phenyl-1-[4-(10-phenylanthracen-9-yl)phenyl]-11H-benzo[a]carbazole (abbreviation: aBCzPAP) represented by Structural Formula (ii) shown above and N,N′-bis (3-methylphenyl)-N,N-bis[3-(9-phenyl-9H-fluoren-9-yl)phenyl]-pyrene-1,6-diamine (abbreviation: 1,6mMemFLPAPrn) represented by Structural Formula (iii) shown above. The light-emitting layer 113 was formed to have a thickness of 25 nm such that the weight ratio of aBCzPAP to 1,6mMemFLPAPrn was 1:0.03.

Then, the electron-transport layer 114 was formed over the light-emitting layer 113 in such a way that a 10-nm-thick film of aBCzPAP was formed and a 15-nm-thick film of bathophenanthroline (abbreviation: BPhen) represented by Structural Formula (iv) shown above was formed.

After the formation of the electron-transport layer 114, lithium fluoride (LiF) was deposited by evaporation to a thickness of 1 nm as the electron-injection layer 115 and aluminum was deposited by evaporation to a thickness of 200 nm as the second electrode 102. Thus, the light-emitting element 1 in this example was fabricated.

The element structure of the light-emitting element 1 is shown in a table below.

TABLE 1

| | Hole- | | | | |
|---|---|---|---|---|---|
| Hole-injection | transport | | Electron-transport | | Electron- |
| layer | layer | Light-emitting layer | layer | | injection layer |
| 10 nm | 30 nm | 25 nm | 10 nm | 15 nm | 1 nm |
| PCzPA:MoOx | PCzPA | aBCzPAP:1, | aBCzPAP | BPhen | LiF |
| (4:2) | | 6mMemFLPAPrn | | | |
| | | (1:0.03) | | | |

The light-emitting element 1 was sealed using a glass substrate in a glove box containing a nitrogen atmosphere so as not to be exposed to the air (specifically, a sealing material was applied to surround the element and UV treatment and heat treatment at 80° C. for 1 hour were performed at the time of sealing). Then, the initial characteristics and reliability of the light-emitting element were measured. Note that the measurements were performed at room temperature (in an atmosphere kept at 25° C.).

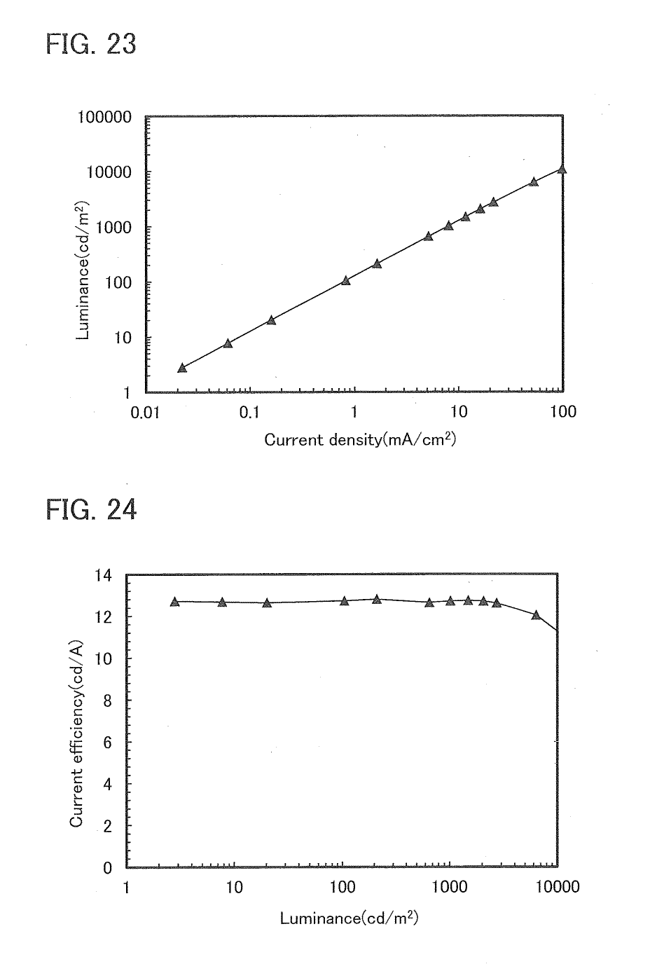

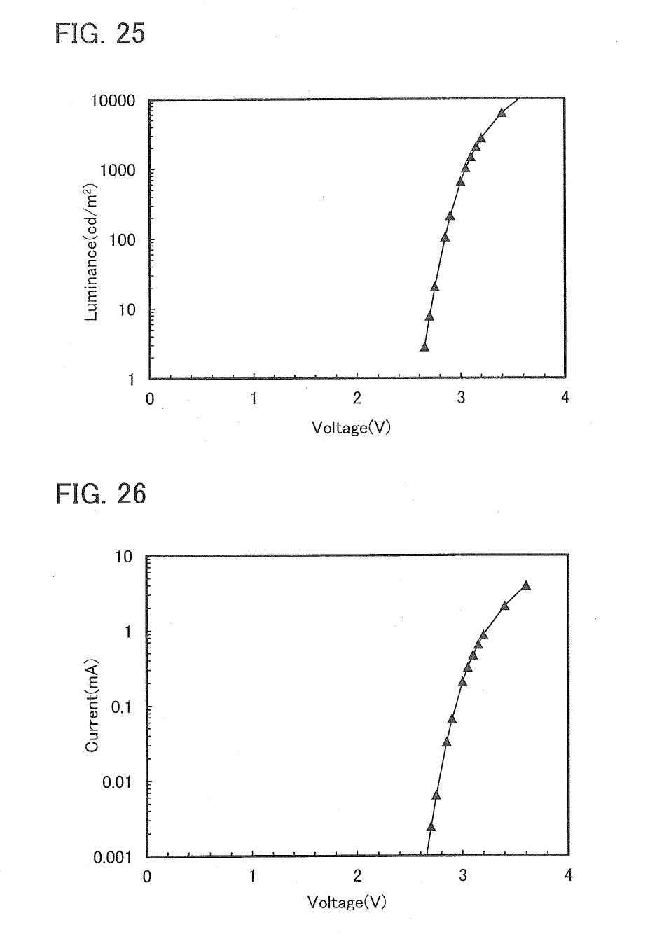

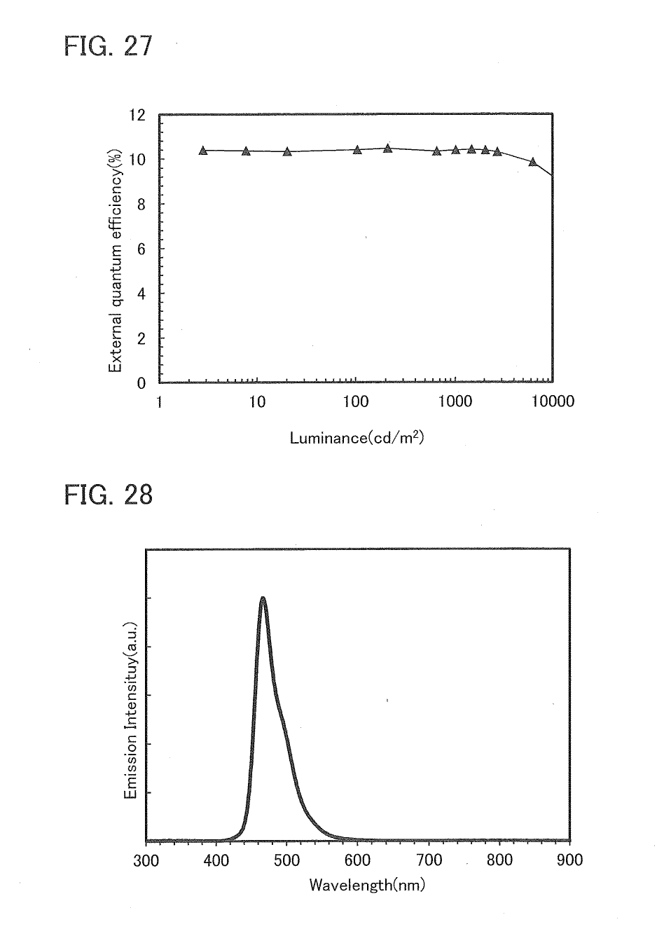

FIG. 23 shows luminance-current density characteristics of the light-emitting element 1. FIG. 24 shows current efficiency-luminance characteristics of the light-emitting element 1. FIG. 25 shows luminance-voltage characteristics of the light-emitting element 1. FIG. 26 shows current-voltage characteristics of the light-emitting element 1. FIG. 27 shows external quantum efficiency-luminance characteristics of the light-emitting element 1. FIG. 28 shows an emission spectrum of the light-emitting element 1. Table 2 shows main characteristics of the light-emitting element 1 at around 1000 cd/m².

TABLE 2

| | | | | | Current | External quantum |
|---|---|---|---|---|---|---|
| Voltage | Current | Current density | | | efficiency | efficiency |
| (V) | (mA) | (mA/cm²) | Chromaticity x | Chromaticity y | (cd/A) | (%) |
| 3.1 | 0.32 | 8 | 0.14 | 0.18 | 12.7 | 10.4 |

It was found from FIGS. 23 to 28 and Table 2 that the light-emitting element of one embodiment of the present invention has favorable characteristics of efficient blue light emission with favorable chromaticity.

### Example 5

In this example, a light-emitting element 2 and a light-emitting element 3 each of which is one embodiment of the present invention and described in Embodiment is described in detail. Structural formulae of organic compounds used for the light-emitting elements 2 and 3 are shown below.

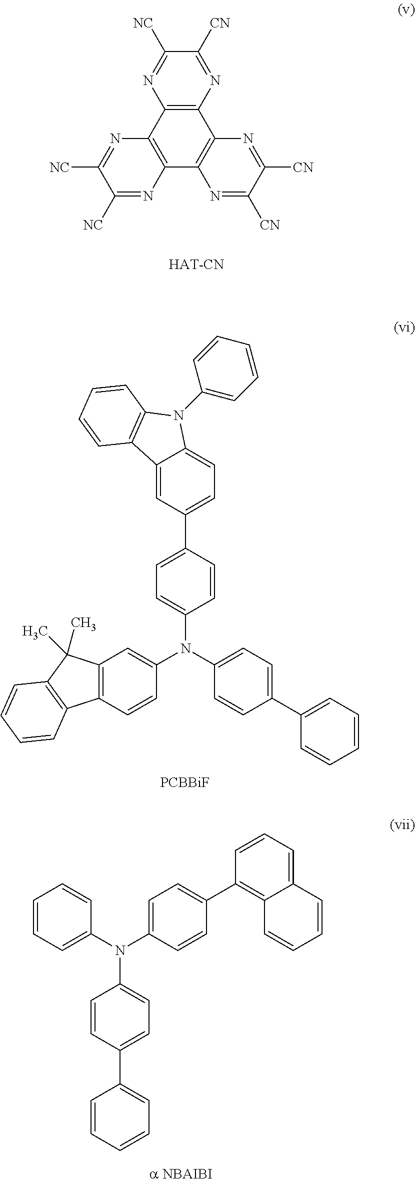

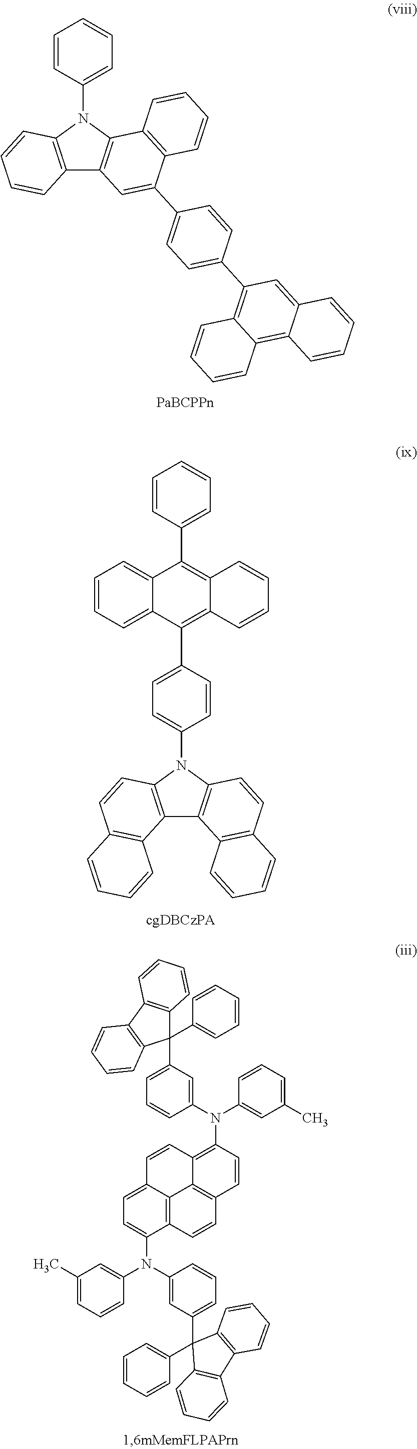

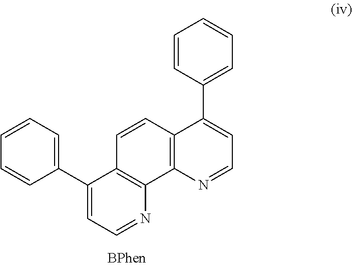 [SMILES: c1ccc(-c2ccnc3c2ccc2c(-c4ccccc4)ccnc23)cc1]

### (Manufacturing Method of Light-Emitting Element 2)

First, indium tin oxide containing silicon oxide (ITSO) was deposited over a glass substrate by a sputtering method, whereby the first electrode 101 was formed. The thickness of the first electrode 101 was set to 70 nm and the area of the electrode was set to 2 mm×2 mm.

Next, in pretreatment for forming the light-emitting element over the substrate, a surface of the substrate was washed with water and baked at 200° C. for 1 hour, and then UV ozone treatment was performed for 370 seconds.

Then, the substrate was transferred into a vacuum evaporation apparatus in which the pressure was reduced to approximately 10⁻⁴ Pa, and vacuum baking at 170° C. for 30 minutes was conducted in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus such that the surface on which the first electrode 101 was formed faced downward. After that, over the first electrode 101, 2,3,6,7,10,11-hexacyano-1,4,5,8,9,12-hexaazatriphenylene (abbreviation: HAT-CN) represented by Structural Formula (v) shown above was deposited to a thickness of 5 nm by an evaporation method using resistance heating, whereby the hole-injection layer 111 was formed.

Next, N-(1,1′-biphenyl-4-yl)-9,9-dimethyl-N-[4-(9-phenyl-9H-carbazol-3-yl)phenyl]-9H-fluor en-2-amine (abbreviation: PCBBiF) represented by Structural Formula (vi) shown above was deposited by evaporation to a thickness of 20 nm over the hole-injection layer 111, whereby the first hole-transport layer was formed; 4-(1-naphthyl)-4′-phenyltriphenylamine (abbreviation: αNBA1BP) represented by Structural Formula (vii) shown above was deposited by evaporation to a thickness of 5 nm over the first hole-transport layer, whereby the second hole-transport layer was formed; and 5-[4-(9-phenanthryl)phenyl]-11-phenyl-11H-benzo[a]carbazole (abbreviation: PaBCPPn) represented by Structural Formula (viii) was deposited by evaporation to a thickness of 5 nm over the second hole-transport layer, whereby the third hole-transport layer was formed.

Next, the light-emitting layer 113 was formed by co-evaporation of 7-[4-(10-phenyl-9-anthryl)phenyl]-7H-dibenzo[c,g]carbazole (abbreviation: cgDBCzPA) represented by Structural Formula (ix) shown above and N,N-bis (3-methylphenyl)-N,N-bis[3-(9-phenyl-9H-fluoren-9-yl)phenyl]-pyrene-1,6-diamine (abbreviation: 1,6mMemFLPAPrn) represented by Structural Formula (iii) shown above. The light-emitting layer 113 was formed to have a thickness of 25 nm such that the weight ratio of cgDBCzPA to 1,6mMemFLPAPrn was 1:0.03.

Then, over the light-emitting layer 113, cgDBCzPA was deposited to a thickness of 10 nm by evaporation, and bathophenanthroline (abbreviation: BPhen) represented by Structural Formula (iv) was deposited to a thickness of 15 nm by evaporation, whereby the electron-transport layer 114 was formed.

After the formation of the electron-transport layer 114, lithium fluoride (LiF) was deposited by evaporation to a thickness of 1 nm as the electron-injection layer 115 and aluminum was deposited by evaporation to a thickness of 200 nm as the second electrode 102. Thus, the light-emitting element 2 in this example was fabricated.

The element structure of the light-emitting element 2 is shown in a table below.

TABLE 3
Hole-

| injection | Hole-transport layer | | | Light-emitting | | | Electron- |
|---|---|---|---|---|---|---|---|
| layer | 1 | 2 | 3 | layer | Electron-transport layer | | injection layer |
| 5 nm | 20 nm | 5 nm | 5 nm | 25 nm | 10 nm | 15 nm | 1 nm |
| HAT-CN | PCBBiF | αNBA1BP | PaBCPPn | cgDBCzPA: | cgDBCzPA | BPhen | LiF |
| | | | | 1,6mMemFLPAPrn | | | |
| | | | | (1:0.03) | | | |

### (Method for Fabricating Light-Emitting Element 3)

The light-emitting element 3 was fabricated by a process that is the same as that for the light-emitting element 2 up to the formation of the first electrode.

After the first electrode 101 was formed, the substrate provided with the first electrode 101 was fixed to a substrate holder provided in the vacuum evaporation device such that the surface on which the first electrode 101 was formed faced downward. By an evaporation method using resistance heating, PaBCPPn and molybdenum(VI) oxide were deposited by co-evaporation over the first electrode 101, whereby the hole-injection layer 111 was formed. The hole-injection layer 111 was formed to a thickness of 10 nm such that the weight ratio of PaBCPPn to molybdenum oxide was 4:2.

Next, PaBCPPn was deposited by evaporation to a thickness of 30 nm over the hole-injection layer 111 to form the hole-transport layer 112.

Next, cgDBCzPA and 1,6mMemFLPAPrn were deposited by co-evaporation to a thickness of 25 nm such that the weight ratio of cgDBCzPA to 1,6mMemFLPAPrn was 1:0.03, whereby the light-emitting layer 113 was formed.

Then, over the light-emitting layer 113, cgDBCzPA was deposited to a thickness of 10 nm by evaporation, and BPhen was deposited to a thickness of 15 nm by evaporation, whereby the electron-transport layer 114 was formed.

After the formation of the electron-transport layer 114, lithium fluoride (LiF) was deposited by evaporation to a thickness of 1 nm as the electron-injection layer 115 and aluminum was deposited by evaporation to a thickness of 200 nm as the second electrode 102. Thus, the light-emitting element 3 in this example was fabricated.

The element structure of the light-emitting element 3 is shown in a table below.

TABLE 4

| Hole- | Hole- | | | | Electron- |
|---|---|---|---|---|---|
| injection | transport | | Electron-transport | | injection |
| layer | layer | Light-emitting layer | layer | | layer |
| 10 nm | 30 nm | 25 nm | 10 nm | 15 nm | 1 nm |
| PaBCPPn:MoOx | PaBCPPn | cgDBCzPA:1, | cgDBCzPA | BPhen | LiF |
| (4:2) | | 6mMemFLPAPrn | | | |
| | | (1:0.03) | | | |

The light-emitting elements 2 and 3 were sealed using a glass substrate in a glove box containing a nitrogen atmosphere so as not to be exposed to the air (specifically, a sealing material was applied to surround the element and UV treatment and heat treatment at 80° C. for 1 hour were performed at the time of sealing). Then, the initial characteristics and reliability of the light-emitting elements were measured. Note that the measurements were performed at room temperature (in an atmosphere kept at 25° C.).

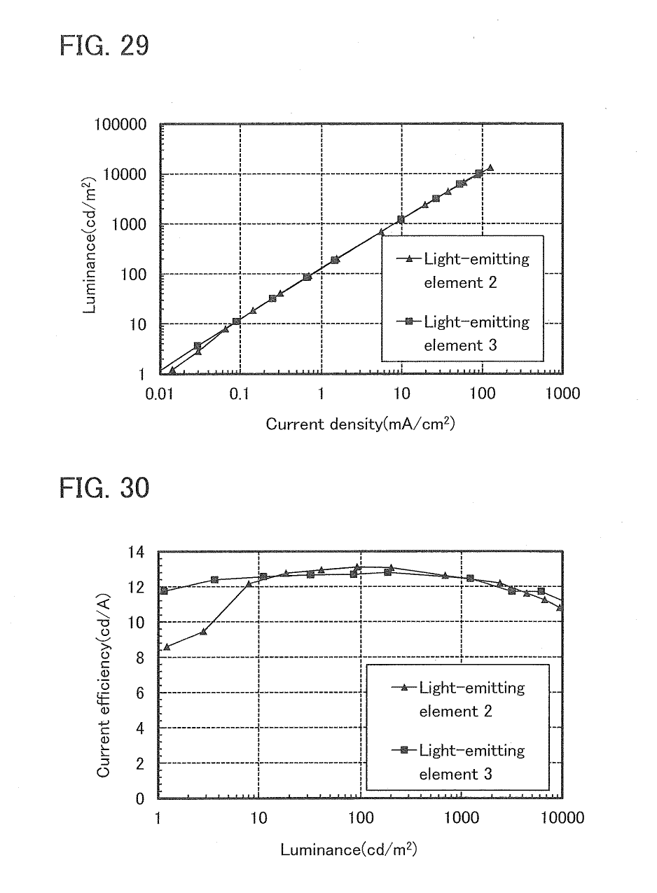

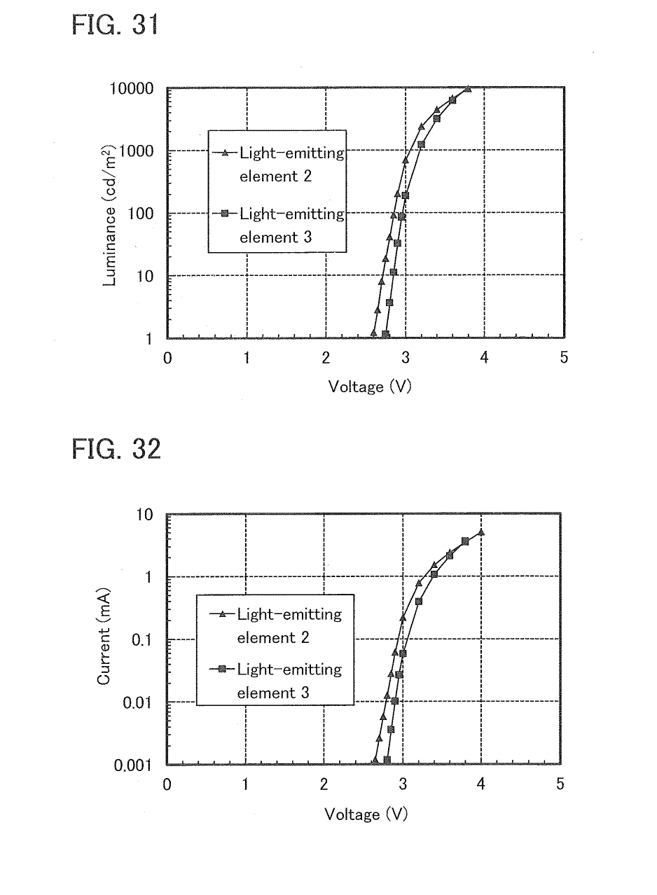

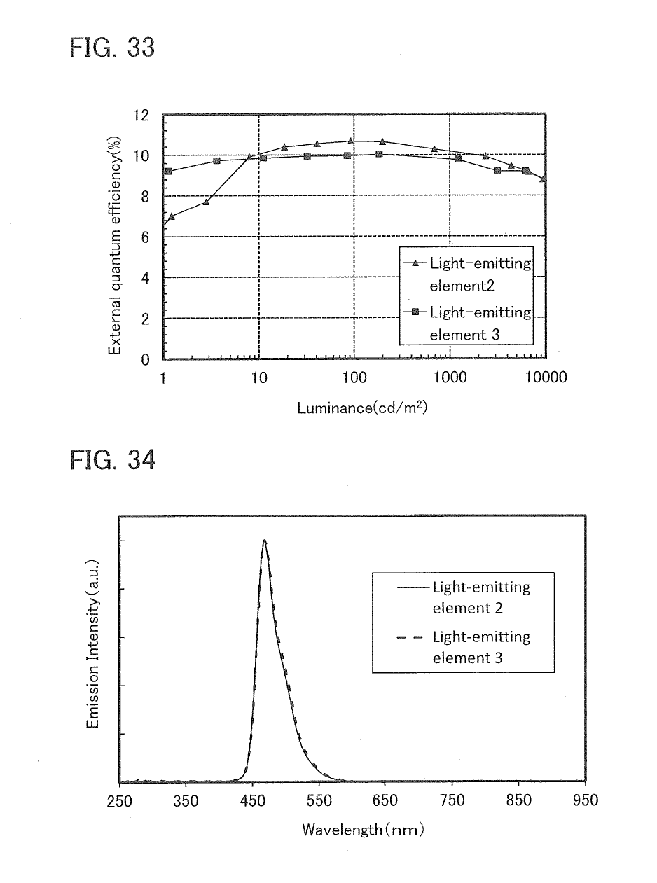

FIG. 29 shows luminance-current density characteristics of the light-emitting elements 2 and 3. FIG. 30 shows current efficiency-luminance characteristics of the light-emitting elements 2 and 3. FIG. 31 shows luminance-voltage characteristics of the light-emitting elements 2 and 3. FIG. 32 shows current-voltage characteristics of the light-emitting elements 2 and 3. FIG. 33 shows external quantum efficiency-luminance characteristics of the light-emitting elements 2 and 3. FIG. 34 shows an emission spectrum of the light-emitting elements 2 and 3. Table 5 shows main characteristics of the light-emitting elements 2 and 3 at around 1000 cd/m². According to FIG. 29 to FIG. 34, the light-emitting elements in which PaBCPPn that is a compound of one embodiment of the present invention was used for the hole-transport layer exhibited excellent characteristics. Therefore, PaBCPPn turned out to have a high hole-transport property.

TABLE 5

| | | | | | | Current | External quantum |
|---|---|---|---|---|---|---|---|
| | Voltage | Current | Current density | | | efficiency | efficiency |
| | (V) | (mA) | (mA/cm²) | Chromaticity x | Chromaticity y | (cd/A) | (%) |
| Light-emitting | 3.0 | 0.22 | 5 | 0.14 | 0.19 | 12.6 | 10.3 |
| element 2 | | | | | | | |
| Light-emitting | 3.2 | 0.39 | 10 | 0.14 | 0.19 | 12.5 | 9.8 |
| element 3 | | | | | | | |

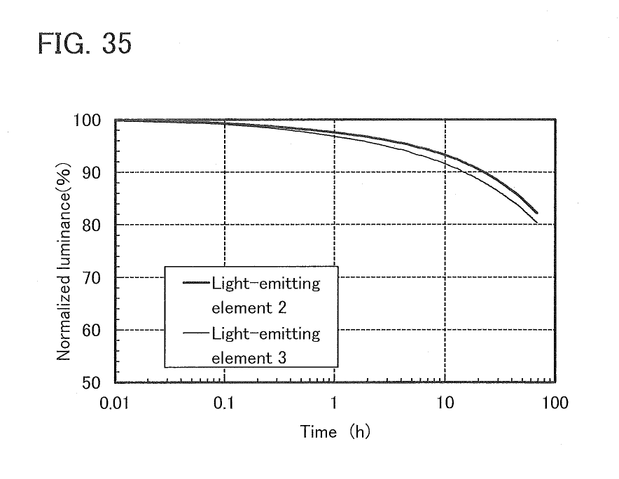

FIG. 35 shows driving time-dependent change in luminance of the light-emitting elements under the conditions where the initial luminance was 5000 cd/m² and the current density was constant. As shown in FIG. 35, it was found that the light-emitting elements 2 and 3 are long-lifetime light-emitting elements in each of which a reduction in luminance with driving time is small.

### Example 6

In this example, a light-emitting element 4 that is a comparative example and a light-emitting element 5 that is one embodiment of the present invention and described in Embodiment are described in detail. Structural formulae of organic compounds used for the light-emitting elements 4 and 5 are shown below.

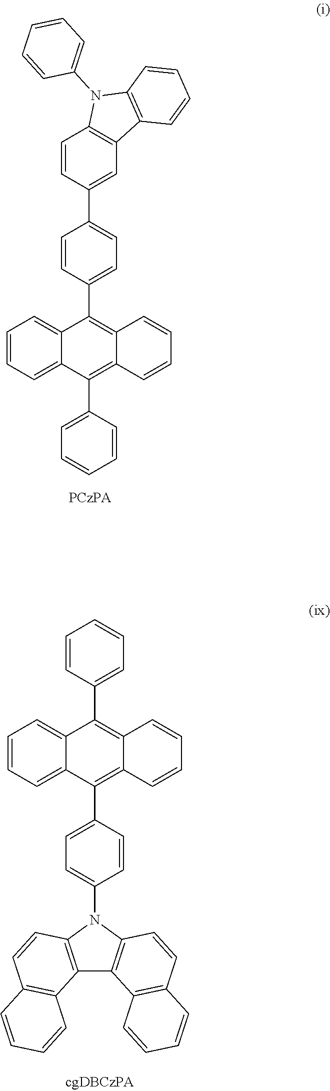 [SMILES: c1ccc(-c2c3ccccc3c(-c3ccc(-c4ccc5c(c4)c4ccccc4n5-c4ccccc4)cc3)c3ccccc23)cc1 ; c1ccc(-c2c3ccccc3c(-c3ccc(-n4c5ccc6ccccc6c5c5c6ccccc6ccc54)cc3)c3ccccc23)cc1]

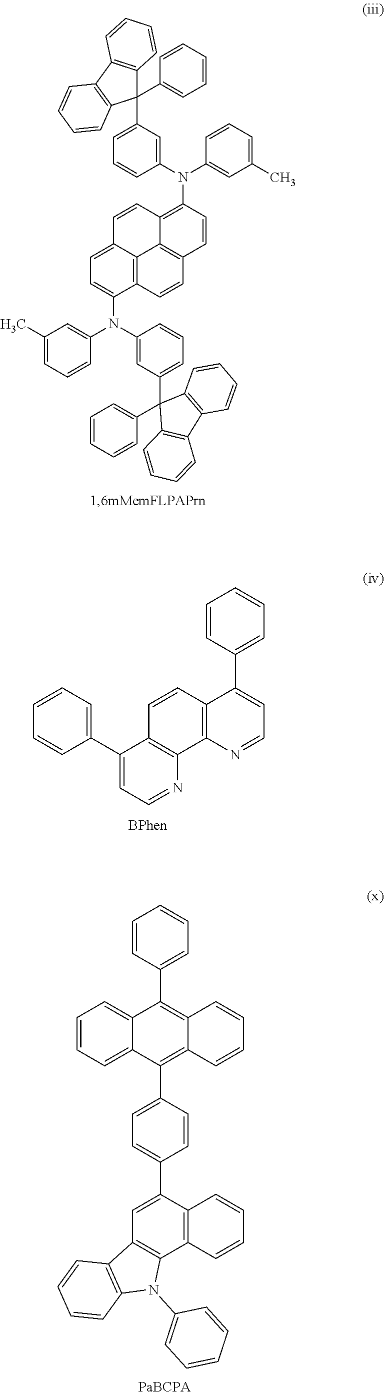

### (Manufacturing Method of Light-Emitting Element 4)

First, indium tin oxide containing silicon oxide (ITSO) was deposited over a glass substrate by a sputtering method, whereby the first electrode 101 was formed. The thickness of the first electrode 101 was set to 70 nm and the area of the electrode was set to 4 mm² (2 mm×2 mm).

Next, in pretreatment for forming the light-emitting element over the substrate, a surface of the substrate was washed with water and baked at 200° C. for 1 hour, and then UV ozone treatment was performed for 370 seconds.

Then, the substrate was transferred into a vacuum evaporation apparatus in which the pressure was reduced to approximately 10⁻⁴ Pa, and vacuum baking at 170° C. for 30 minutes was conducted in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus such that the surface on which the first electrode 101 was formed faced downward. After that, over the first electrode 101, 9-phenyl-3-[4-(10-phenyl-9-anthryl)phenyl]-9H-carabzole (abbreviation: PCzPA) represented by Structural Formula (i) shown above and molybdenum(VI) oxide were co-evaporated to a thickness of 10 nm by an evaporation method using resistance heating such that the weight ratio of PCzPA to molybdenum oxide was 4:2, whereby the hole-injection layer 111 was formed.

Next, PCzPA was deposited by evaporation to a thickness of 30 nm over the hole-injection layer 111 to form the hole-transport layer.

Next, the light-emitting layer 113 was formed by co-evaporation of 7-[4-(10-phenyl-9-anthryl)phenyl]-7H-dibenzo[c,g]carbazole (abbreviation: cgDBCzPA) represented by Structural Formula (ix) shown above and N,N-bis (3-methylphenyl)-N,N-bis[3-(9-phenyl-9H-fluoren-9-yl)phenyl]-pyrene-1,6-diamine (abbreviation: 1,6mMemFLPAPrn) represented by Structural Formula (iii) shown above. The light-emitting layer 113 was formed to have a thickness of 25 nm such that the weight ratio of cgDBCzPA to 1,6mMemFLPAPm was 1:0.03.

Then, over the light-emitting layer 113, cgDBCzPA was deposited to a thickness of 10 nm by evaporation, and bathophenanthroline (abbreviation: BPhen) represented by Structural Formula (iv) was deposited to a thickness of 15 nm by evaporation, whereby the electron-transport layer 114 was formed.

After the formation of the electron-transport layer 114, lithium fluoride (LiF) was deposited by evaporation to a thickness of 1 nm as the electron-injection layer 115 and aluminum was deposited by evaporation to a thickness of 200 nm as the second electrode 102. Thus, the light-emitting element 4 that was a comparative example was fabricated.

### (Manufacturing Method of Light-Emitting Element 5)

The light-emitting element 5 was fabricated in a manner similar to that of the light-emitting element 4 except that PCzPA in the light-emitting element 4 was replaced with 11-phenyl-5-[4-(10-phenylanthracen-9-yl)phenyl]-11H-benzo[a]carbazole (abbreviation: PaBCPA) represented by Structural Formula (x) shown above.

The element structure of the light-emitting element 4 is shown in Table 6, and the element structure of the light-emitting element 5 is shown in Table 7.

TABLE 6

| | | | | | Electron- |
|---|---|---|---|---|---|
| Hole-injection | Hole-transport | | Electron-transport | | injection |
| layer | layer | Light-emitting layer | layer | | layer |
| 10 nm | 30 nm | 25 nm | 10 nm | 15 nm | 1 nm |
| PCzPA:MoOx | PCzPA | cgDBCzPA:1, | cgDBCzPA | BPhen | LiF |
| (4:2) | | 6mMemFLPAPrn | | | |
| | | (1:0.03) | | | |

TABLE 7

| | | | | | Electron- |
|---|---|---|---|---|---|
| Hole-injection | Hole-transport | | Electron-transport | | injection |
| layer | layer | Light-emitting layer | layer | | layer |
| 10 nm | 30 nm | 25 nm | 10 nm | 15 nm | 1 nm |
| PaBCPA:MoOx | PaBCPA | cgDBCzPA:1, | cgDBCzPA | BPhen | LiF |
| (4:2) | | 6MemFLPAPrn | | | |
| | | (1:0.03) | | | |

The light-emitting elements 4 and 5 were sealed using a glass substrate in a glove box containing a nitrogen atmosphere so as not to be exposed to the air (specifically, a sealing material was applied to surround the element and UV treatment and heat treatment at 80° C. for 1 hour were performed at the time of sealing). Then, the initial characteristics and reliability of the light-emitting elements were measured. Note that the measurements were performed at room temperature (in an atmosphere kept at 25° C.).

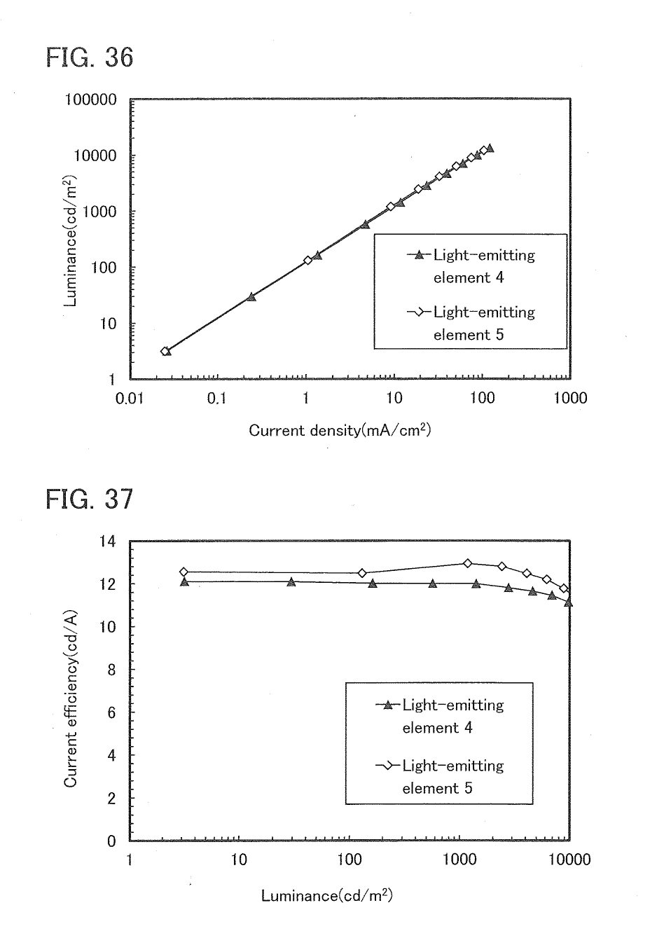

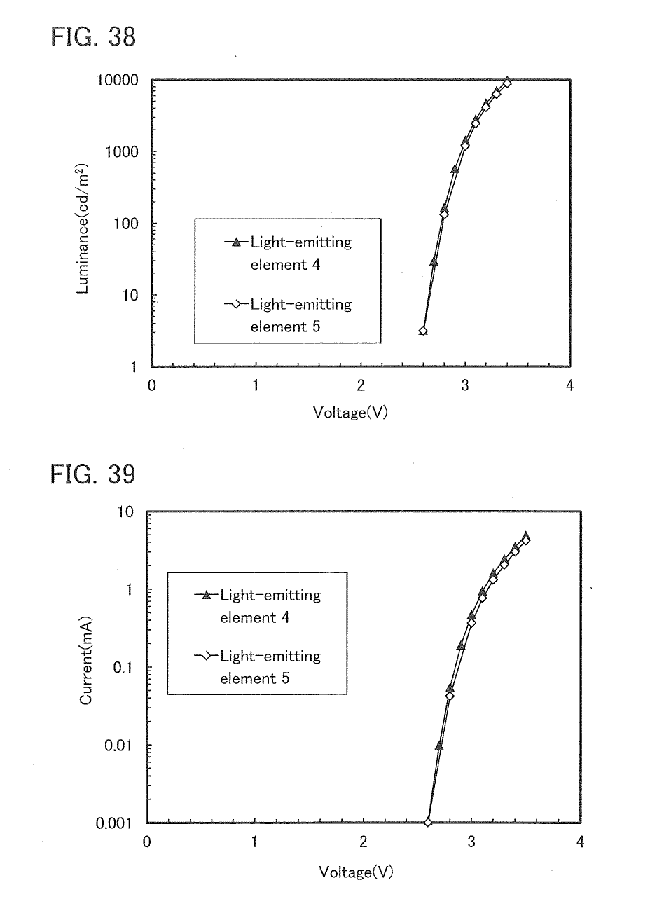

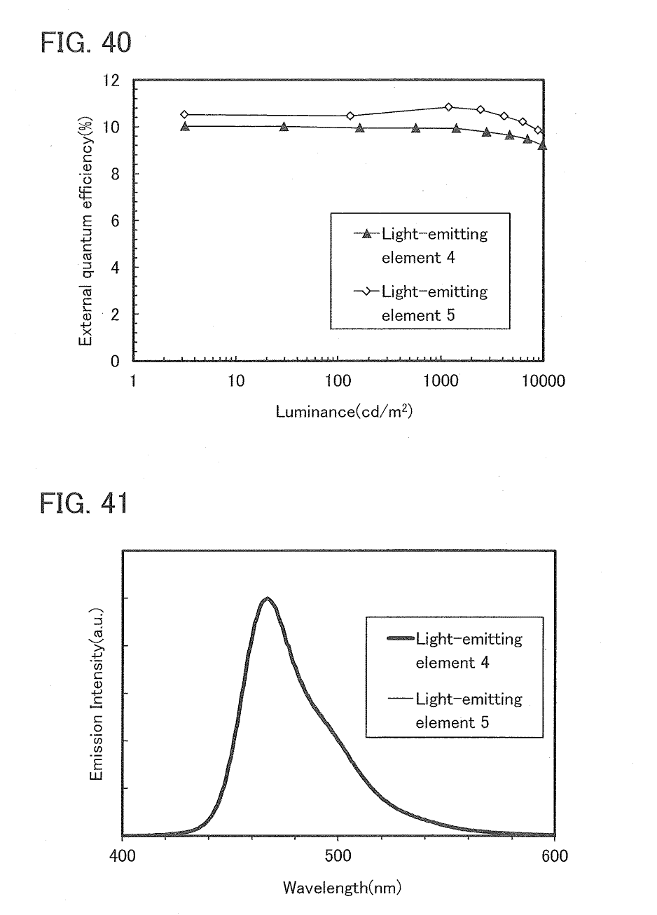

FIG. 36 shows luminance-current density characteristics of the light-emitting elements 4 and 5. FIG. 37 shows current efficiency-luminance characteristics of the light-emitting elements 4 and 5. FIG. 38 shows luminance-voltage characteristics of the light-emitting elements 4 and 5. FIG. 39 shows current-voltage characteristics of the light-emitting elements 4 and 5. FIG. 40 shows external quantum efficiency-luminance characteristics of the light-emitting elements 4 and 5. FIG. 41 shows an emission spectrum of the light-emitting elements 4 and 5. Table 8 shows main characteristics of the light-emitting elements 4 and 5 at around 1000 cd/m². According to FIG. 36 to FIG. 41 and Table 8, the light-emitting element 5 in which PaBCPA that is a compound of one embodiment of the present invention was used for the hole-transport layer exhibited excellent characteristics. Therefore, PaBCPA turned out to have a high hole-transport property.

TABLE 8

| | | | | | | Current | External quantum |
|---|---|---|---|---|---|---|---|
| | Voltage | Current | Current density | | | efficiency | efficiency |
| | (V) | (mA) | (mA/cm ) | Chromaticity x | Chromaticity y | (cd/A) | (%) |
| Light-emitting | 3.0 | 0.47 | 12 | 0.14 | 0.17 | 12.0 | 9.9 |
| element 4 | | | | | | | |
| Light-emitting | 3.0 | 0.37 | 9 | 0.14 | 0.17 | 12.9 | 10.8 |
| element 5 | | | | | | | |

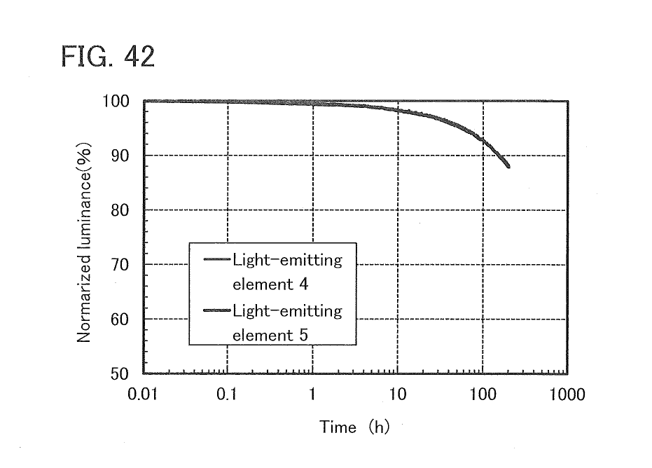

FIG. 42 shows driving time-dependent change in luminance of the light-emitting elements under the conditions where the current value was 2 mA and the current density was constant. As shown in FIG. 42, it was found that the light-emitting element 5 is a long-lifetime light-emitting element in which a reduction in luminance with driving time is small.

### Example 7

In this example, a light-emitting element 6 which is one embodiment of the present invention and described in Embodiment is described in detail. Structural formulae of organic compounds used for the light-emitting element 6 are shown below.

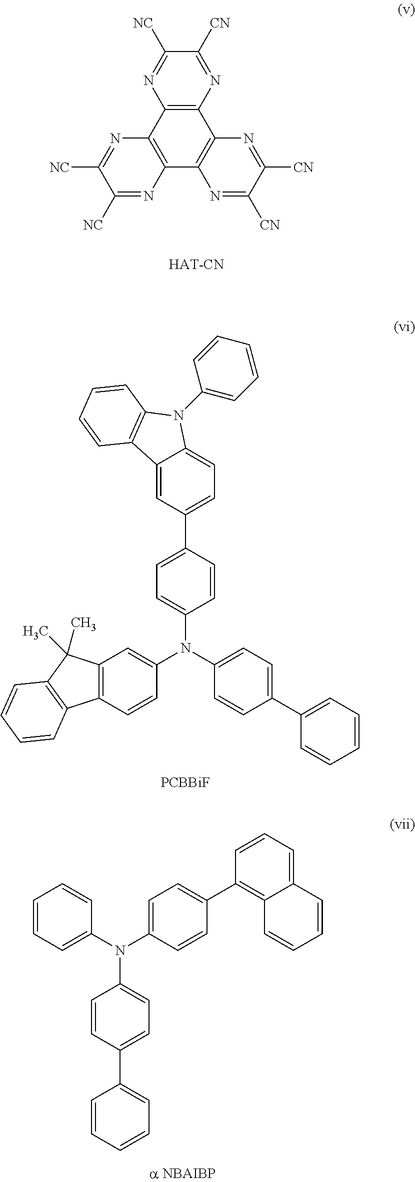

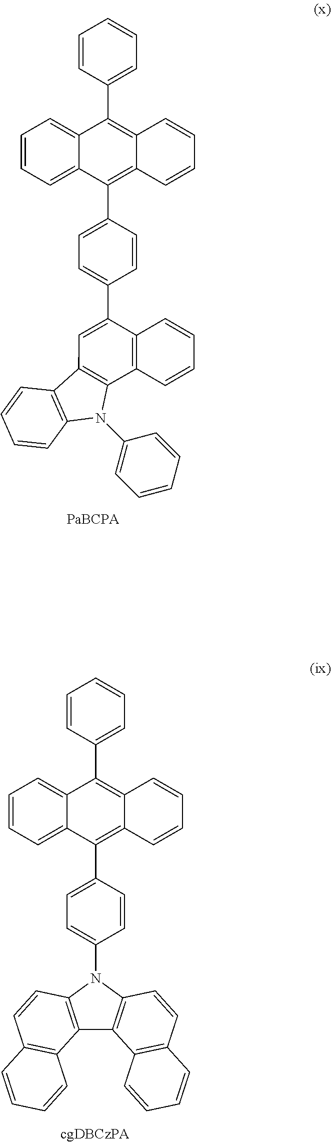 [SMILES: c1ccc(-c2c3ccccc3c(-c3ccc(-c4cc5c6ccccc6n(-c6ccccc6)c5c5ccccc45)cc3)c3ccccc23)cc1 ; c1ccc(-c2c3ccccc3c(-c3ccc(-n4c5ccc6ccccc6c5c5c6ccccc6ccc54)cc3)c3ccccc23)cc1]

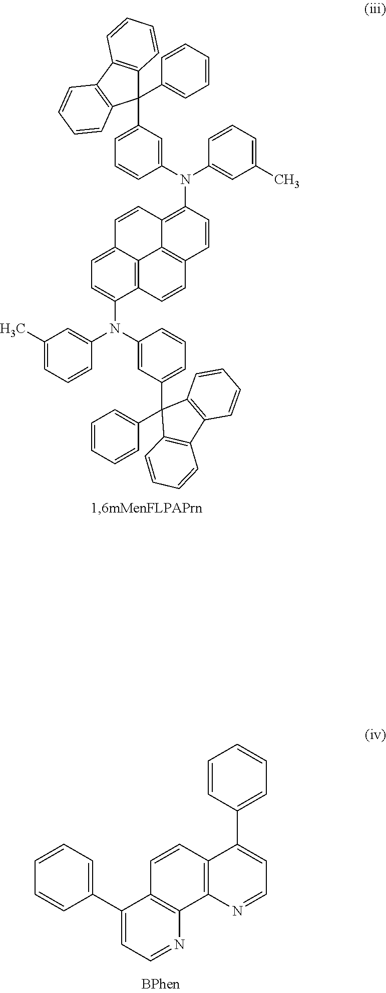

### (Manufacturing Method of Light-Emitting Element 6)

First, indium tin oxide containing silicon oxide (ITSO) was deposited over a glass substrate by a sputtering method, whereby the first electrode 101 was formed. The thickness of the first electrode 101 was set to 70 nm and the area of the electrode was set to 4 mm² (2 mm×2 mm).

Next, in pretreatment for forming the light-emitting element over the substrate, a surface of the substrate was washed with water and baked at 200° C. for 1 hour, and then UV ozone treatment was performed for 370 seconds.

Then, the substrate was transferred into a vacuum evaporation apparatus in which the pressure was reduced to approximately 10⁻⁴ Pa, and vacuum baking at 170° C. for 30 minutes was conducted in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus such that the surface on which the first electrode 101 was formed faced downward. After that, over the first electrode 101, 2,3,6,7,10,11-hexacyano-1,4,5,8,9,12-hexaazatriphenylene (abbreviation: HAT-CN) represented by Structural Formula (v) shown above was deposited to a thickness of 5 nm by an evaporation method using resistance heating, whereby the hole-injection layer 111 was formed.

Next, N-(1,1′-biphenyl-4-yl)-9,9-dimethyl-N-[4-(9-phenyl-9H-carbazol-3-yl)phenyl]-9H-fluor en-2-amine (abbreviation: PCBBiF) represented by Structural Formula (vi) shown above was deposited by evaporation to a thickness of 10 nm over the hole-injection layer 111, whereby the first hole-transport layer was formed; 4-(1-naphthyl)-4′-phenyltriphenylamine (abbreviation: αNBA1BP) represented by Structural Formula (vii) shown above was deposited by evaporation to a thickness of 10 nm over the first hole-transport layer, whereby the second hole-transport layer was formed; and 11-phenyl-5-[4-(10-phenylanthracen-9-yl)phenyl]-11H-benzo[a]carbazole (abbreviation: PaBCPA) represented by Structural Formula (x) was deposited by evaporation to a thickness of 10 nm over the second hole-transport layer, whereby the third hole-transport layer was formed.

Next, the light-emitting layer 113 was formed by co-evaporation of 7-[4-(10-phenyl-9-anthryl)phenyl]-7H-dibenzo[c,g]carbazole (abbreviation: cgDBCzPA) represented by Structural Formula (ix) shown above and N,N′-bis (3-methylphenyl)-N,N′-bis[3-(9-phenyl-9H-fluoren-9-yl)phenyl]-pyrene-1,6-diamine (abbreviation: 1,6mMemFLPAPm) represented by Structural Formula (iii) shown above. The light-emitting layer 113 was formed to have a thickness of 25 nm such that the weight ratio of cgDBCzPA to 1,6mMemFLPAPrn was 1:0.03.

Then, over the light-emitting layer 113, cgDBCzPA was deposited to a thickness of 10 nm by evaporation, and bathophenanthroline (abbreviation: BPhen) represented by Structural Formula (iv) was deposited to a thickness of 15 nm by evaporation, whereby the electron-transport layer 114 was formed.

After the formation of the electron-transport layer 114, lithium fluoride (LiF) was deposited by evaporation to a thickness of 1 nm as the electron-injection layer 115 and aluminum was deposited by evaporation to a thickness of 200 nm as the second electrode 102. Thus, the light-emitting element 6 in this example was fabricated.

Table 9 shows the element structure of the light-emitting element 6.

TABLE 9

| Hole- | | | | | | | Electron- |
|---|---|---|---|---|---|---|---|
| injection | Hole-transport layer | | | Light-emitting | | | injection |
| layer | 1 | 2 | 3 | layer | Electron-transport layer | | layer |
| 5 nm | 10 nm | 10 nm | 10 nm | 25 nm | 10 nm | 15 nm | 1 nm |
| HAT-CN | PCBBiF | αNBA1BP | PaBCPA | cgDBCzPA: | cgDBCzPA | BPhen | LiF |
| | | | | 1,6mMemFLPAPrn | | | |
| | | | | (1:0.03) | | | |

The light-emitting element 6 was sealed using a glass substrate in a glove box containing a nitrogen atmosphere so as not to be exposed to the air (specifically, a sealing material was applied to surround the element and UV treatment and heat treatment at 80° C. for 1 hour were performed at the time of sealing). Then, the initial characteristics and reliability of the light-emitting element were measured. Note that the measurements were performed at room temperature (in an atmosphere kept at 25° C.).

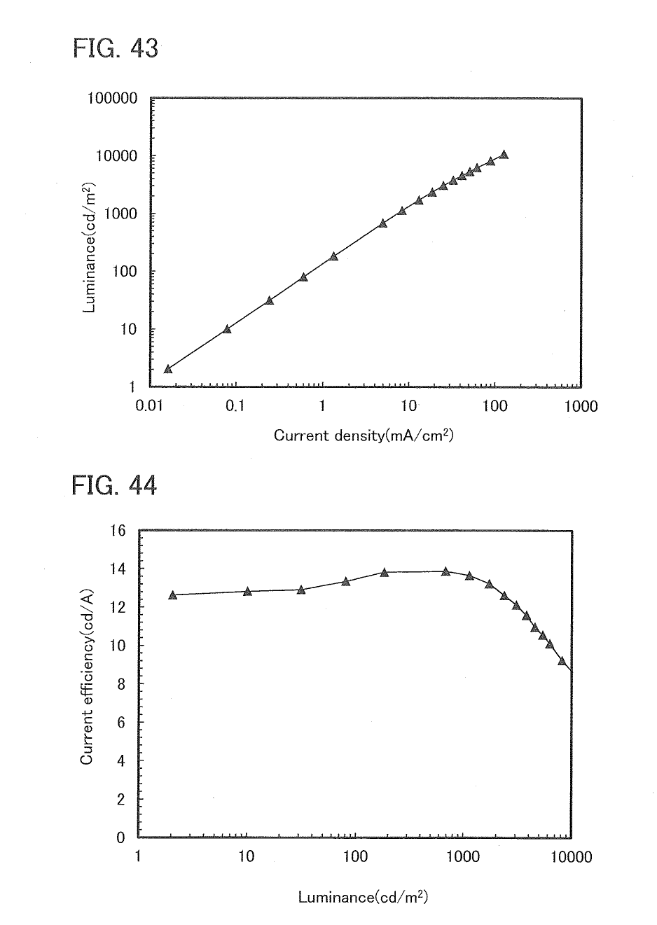

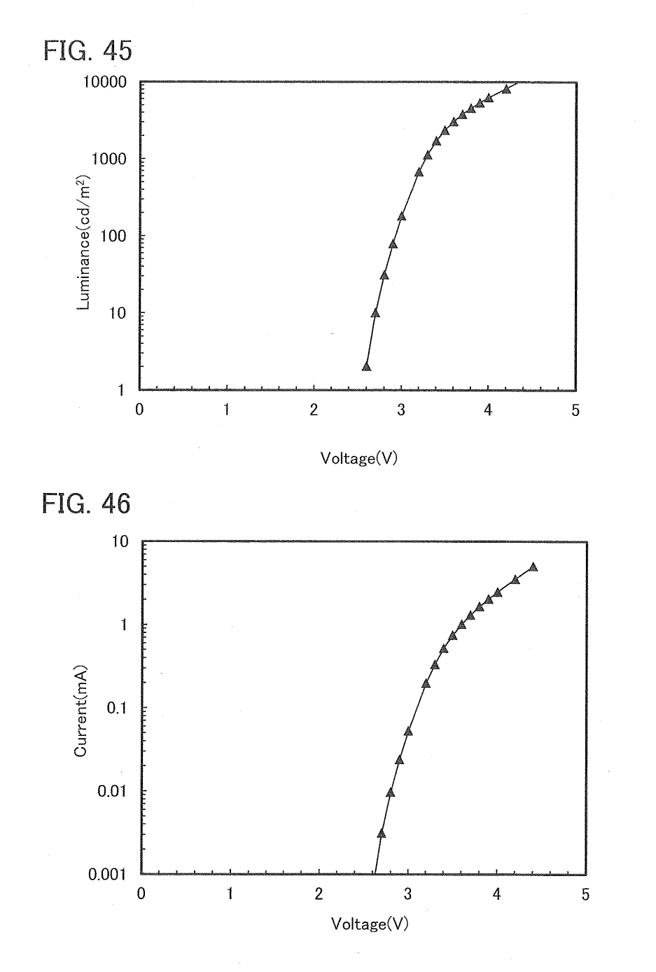

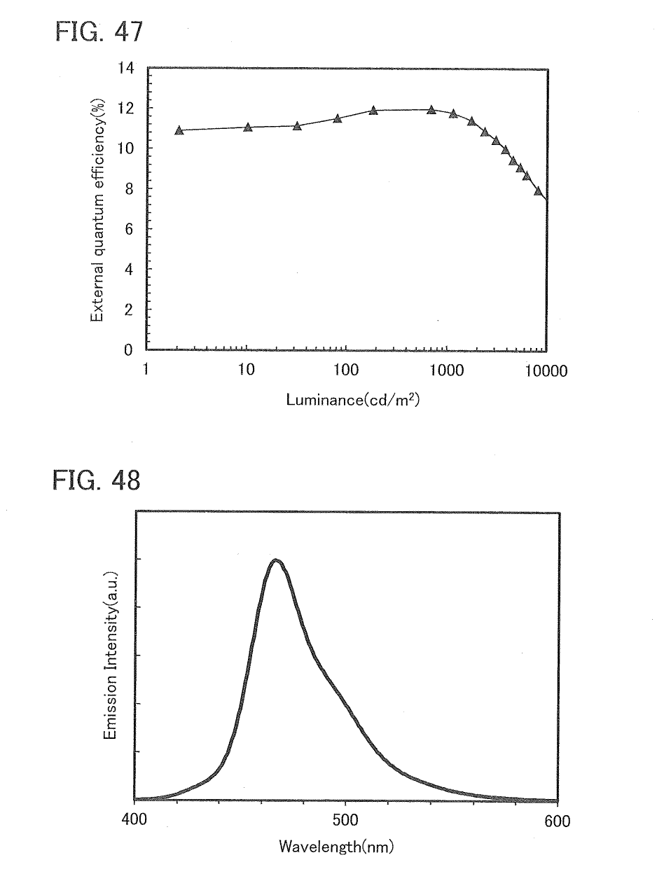

FIG. 43 shows luminance-current density characteristics of the light-emitting element 6. FIG. 44 shows current efficiency-luminance characteristics of the light-emitting element 6. FIG. 45 shows luminance-voltage characteristics of the light-emitting element 6. FIG. 46 shows current-voltage characteristics of the light-emitting element 6. FIG. 47 shows external quantum efficiency-luminance characteristics of the light-emitting element 6. FIG. 48 shows an emission spectrum of the light-emitting element 6. Table 10 shows main characteristics of the light-emitting element 6 at around 1000 cd/m². According to FIG. 43 to FIG. 48, the light-emitting element in which PaBCPA that is a compound of one embodiment of the present invention was used for the hole-transport layer exhibited excellent characteristics. Therefore, PaBCPA turned out to have a high hole-transport property.

TABLE 10

| | | | | | Current | External quantum |
|---|---|---|---|---|---|---|
| Voltage | Current | Current density | | | efficiency | efficiency |
| (V) | (mA) | (mA/cm²) | Chromaticity x | Chromaticity y | (cd/A) | (%) |
| 3.3 | 0.33 | 8 | 0.14 | 0.16 | 13.7 | 11.8 |

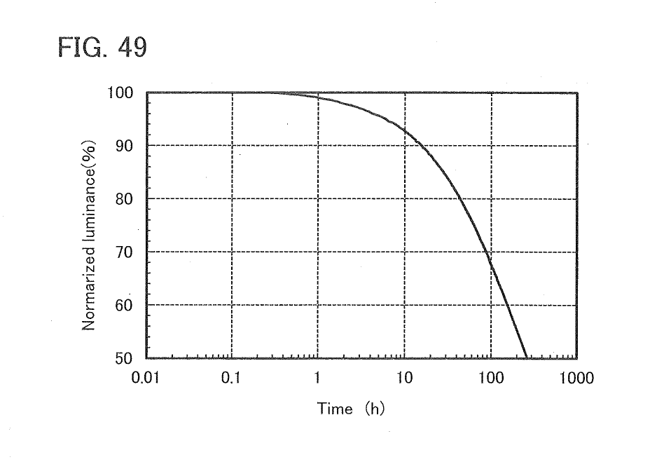

FIG. 49 shows driving time-dependent change in luminance of the light-emitting element under the conditions where the current value was 2 mA and the current density was constant. As shown in FIG. 49, it was found that the light-emitting element 6 is a long-lifetime light-emitting element in which a reduction in luminance with driving time is small.

This application is based on Japanese Patent Application serial no. 2015-214211 filed with Japan Patent Office on Oct. 30, 2015, the entire contents of which are hereby incorporated by reference.

What is claimed is:

1. A light-emitting element material comprising an organic compound represented by General Formula (G1): 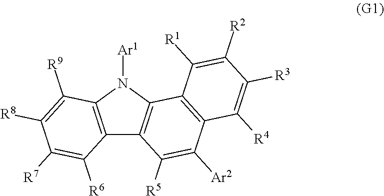
wherein:
Ar¹ represents a substituted or unsubstituted aromatic hydrocarbon group having 6 to 30 carbon atoms;
Ar² represents a substituted or unsubstituted aromatic hydrocarbon group having 6 to 100 carbon atoms;
each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
each of R⁶ to R⁹ independently represents any of hydrogen, a saturated hydrocarbon group having 1 to 6 carbon atoms, and a cyclic saturated hydrocarbon group having 3 to 6 carbon atoms.

2. The light-emitting element material according to claim 1,
wherein the organic compound is represented by General Formula (G2): 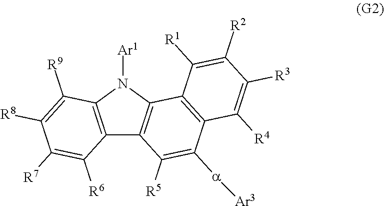
wherein:
α represents a substituted or unsubstituted divalent aromatic hydrocarbon group having 6 to 13 carbon atoms;
Ar³ represents a substituted or unsubstituted aromatic hydrocarbon group having 6 to 50 carbon atoms; and
the substituted or unsubstituted aromatic hydrocarbon group having 6 to 50 carbon atoms comprises a monocyclic aromatic hydrocarbon skeleton or a fused polycyclic aromatic hydrocarbon skeleton having 2 to 12 rings.

3. The light-emitting element material according to claim 1,
wherein the organic compound is represented by General Formula (G3): 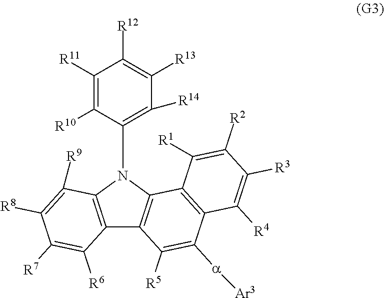
wherein:
α represents a substituted or unsubstituted divalent aromatic hydrocarbon group having 6 to 13 carbon atoms;
Ar³ represents a substituted or unsubstituted aromatic hydrocarbon group having 6 to 50 carbon atoms;
the substituted or unsubstituted aromatic hydrocarbon group having 6 to 50 carbon atoms comprises a monocyclic aromatic hydrocarbon skeleton or a fused polycyclic aromatic hydrocarbon skeleton having 2 to 12 rings; and
each of R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

4. The light-emitting element material according to claim 1,
wherein the organic compound is represented by General Formula (G4): 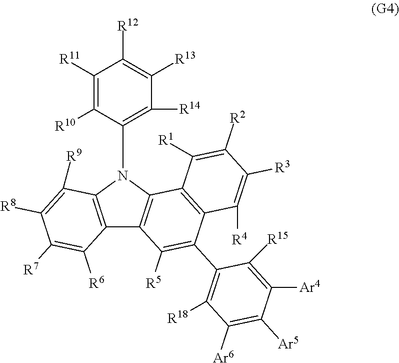
wherein:
any one of Ar⁴ to Ar⁶ represents a substituted or unsubstituted aromatic hydrocarbon group having 6 to 50 carbon atoms;
the substituted or unsubstituted aromatic hydrocarbon group having 6 to 50 carbon atoms comprises a monocyclic aromatic hydrocarbon skeleton or a fused polycyclic aromatic hydrocarbon skeleton having 2 to 12 rings;
each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
each of R¹⁰ to R¹⁵ and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

5. An organic compound represented by General Formula (G1): 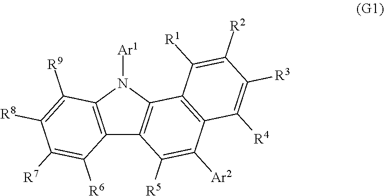
wherein:
Ar¹ represents a substituted or unsubstituted aromatic hydrocarbon group having 6 to 30 carbon atoms;
Ar² represents a substituted or unsubstituted aromatic hydrocarbon group having 6 to 100 carbon atoms;
each of R¹ to R⁵ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
each of R⁶ to R⁹ independently represents any of hydrogen, a saturated hydrocarbon group having 1 to 6 carbon atoms, and a cyclic saturated hydrocarbon group having 3 to 6 carbon atoms.

6. The organic compound according to claim 5, wherein the total number of carbon atoms in Ar¹ and Ar² is greater than or equal to 19 when a fused polycyclic aromatic hydrocarbon skeleton is included in neither Ar¹ nor Ar².

7. The organic compound according to claim 5, wherein the organic compound is represented by General Formula (G2): 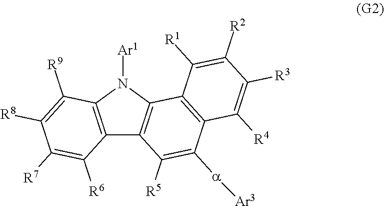
wherein:
α represents a substituted or unsubstituted divalent aromatic hydrocarbon group having 6 to 13 carbon atoms;
Ar³ represents a substituted or unsubstituted aromatic hydrocarbon group having 6 to 50 carbon atoms;
the substituted or unsubstituted aromatic hydrocarbon group having 6 to 50 carbon atoms comprises a monocyclic aromatic hydrocarbon skeleton or a fused polycyclic aromatic hydrocarbon skeleton having 2 to 12 rings; and
the total number of carbon atoms in Ar¹, α, and Ar³ is greater than or equal to 19 when a fused polycyclic aromatic hydrocarbon skeleton is included in neither Ar¹, α, nor Ar³.

8. The organic compound according to claim 7,
wherein the fused polycyclic aromatic hydrocarbon skeleton having 2 to 12 rings is any one of a naphthalene skeleton, an anthracene skeleton, a phenanthrene skeleton, a fluorene skeleton, a pyrene skeleton, a tetracene skeleton, a tetraphene skeleton, a triphenylene skeleton, a chrysene skeleton, and a fluoranthene skeleton.

9. The organic compound according to claim 5, wherein the organic compound is represented by General Formula (G2): 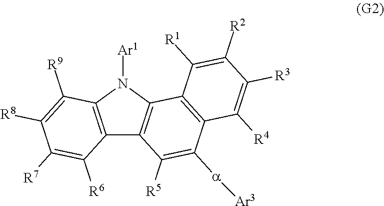
wherein:
α represents a substituted or unsubstituted divalent aromatic hydrocarbon group having 6 to 13 carbon atoms;
Ar³ represents a substituted or unsubstituted aromatic hydrocarbon group having 10 to 30 carbon atoms; and
the substituted or unsubstituted aromatic hydrocarbon group having 10 to 30 carbon atoms comprises a fused polycyclic aromatic hydrocarbon skeleton having 2 to 12 rings.

10. The organic compound according to claim 5, wherein the organic compound is represented by General Formula (G3): 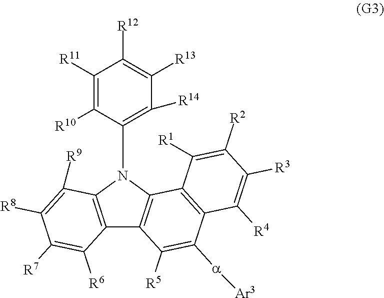
wherein:
α represents a substituted or unsubstituted divalent aromatic hydrocarbon group having 6 to 13 carbon atoms;
Ar³ represents a substituted or unsubstituted aromatic hydrocarbon group having 6 to 50 carbon atoms;
the substituted or unsubstituted aromatic hydrocarbon group having 6 to 50 carbon atoms comprises a monocyclic aromatic hydrocarbon skeleton or a fused polycyclic aromatic hydrocarbon skeleton having 2 to 12 rings;
each of R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
the total number of carbon atoms in α and Ar³ is greater than or equal to 13 when a fused polycyclic aromatic hydrocarbon skeleton is included in neither α nor Ar³.

11. The organic compound according to claim 5, wherein the organic compound is represented by General Formula (G3): 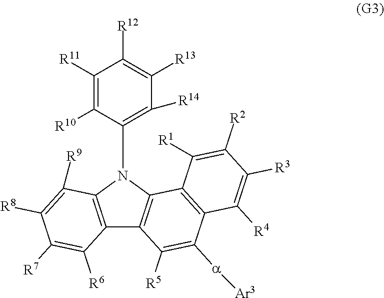
wherein:
α represents a substituted or unsubstituted divalent aromatic hydrocarbon group having 6 to 13 carbon atoms;
Ar³ represents a substituted or unsubstituted aromatic hydrocarbon group having 10 to 30 carbon atoms;
the substituted or unsubstituted aromatic hydrocarbon group having 10 to 30 carbon atoms comprises a fused polycyclic aromatic hydrocarbon skeleton having 2 to 12 rings; and
each of R¹⁰ to R¹⁴ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

12. The organic compound according to claim 5, wherein the organic compound is represented by General Formula (G4): 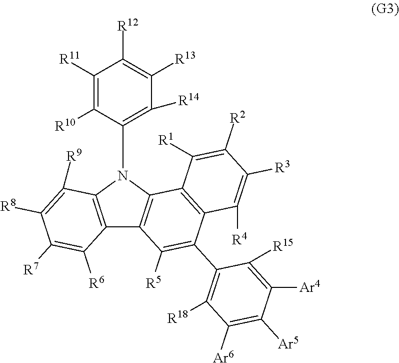
wherein:
any one of Ar⁴ to Ar⁶ represents a substituted or unsubstituted aromatic hydrocarbon group having 10 to 50 carbon atoms;
the substituted or unsubstituted aromatic hydrocarbon group having 10 to 50 carbon atoms comprises a monocyclic aromatic hydrocarbon skeleton or a fused polycyclic aromatic hydrocarbon skeleton having 2 to 12 rings;
each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
each of R¹⁰ to R¹⁵ and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

13. The organic compound according to claim 5, wherein the organic compound is represented by General Formula (G4): 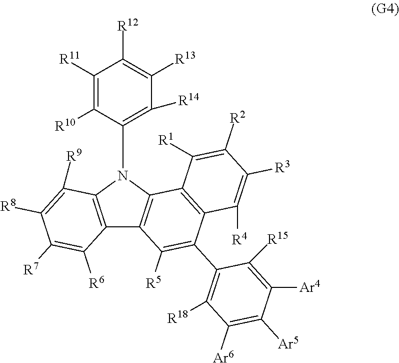
wherein:
any one of Ar⁴ to Ar⁶ represents a substituted or unsubstituted aromatic hydrocarbon group having 10 to 30 carbon atoms;
the substituted or unsubstituted aromatic hydrocarbon group having 10 to 30 carbon atoms comprises a fused polycyclic aromatic hydrocarbon skeleton having 2 to 12 rings;
each of the other two of Ar⁴ to Ar⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms; and
each of R¹⁰ to R¹⁵ and R¹⁸ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

14. The organic compound according to claim 5, wherein the organic compound is represented by General Formula (G5): 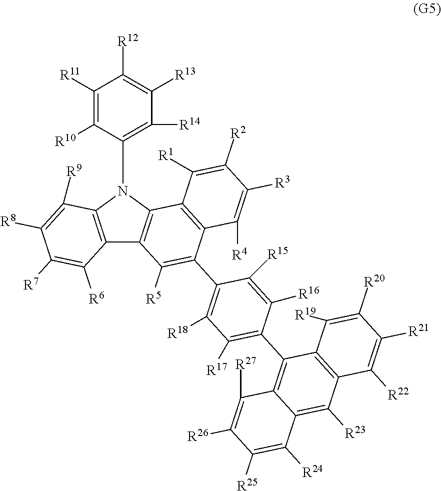
wherein:
each of R¹⁰ to R²⁷ independently represents any of hydrogen, a saturated hydrocarbon group having 1 to 6 carbon atoms, and a substituted or unsubstituted aromatic hydrocarbon group having 6 to 13 carbon atoms.

15. The organic compound according to claim 5, wherein the organic compound is represented by General Formula (G6): 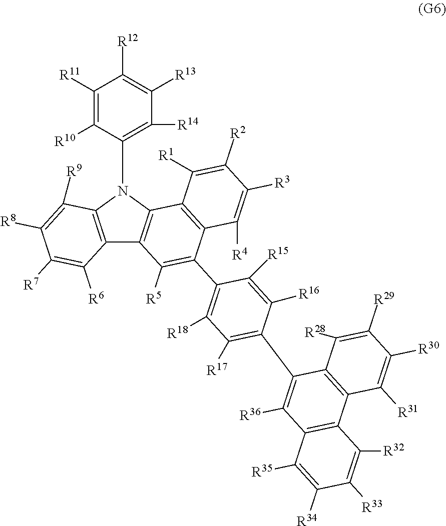
wherein:
each of R¹⁰ to R¹⁸ and R²⁸ to R³⁶ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

16. The organic compound according to claim 5, wherein the organic compound is represented by General Formula (G7): 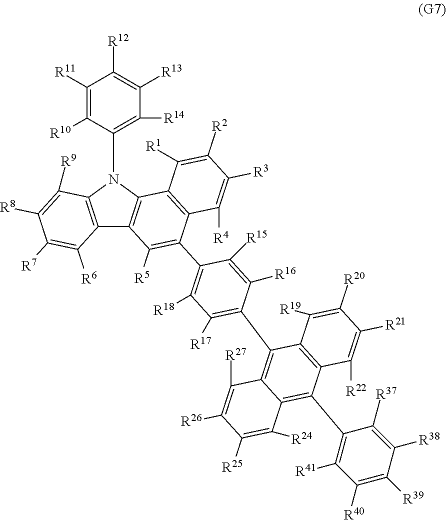
wherein:
each of R¹⁰ to R²², R²⁴ to R²⁷, and R³⁷ to R⁴¹ independently represents any of hydrogen, a saturated hydrocarbon group having 1 to 6 carbon atoms, a cyclic saturated hydrocarbon group having 3 to 6 carbon atoms, and a substituted or unsubstituted aromatic hydrocarbon group having 6 to 13 carbon atoms.

17. The organic compound according to claim 5, wherein the organic compound is represented by any one of Structural Formulae (100), (126), and (136):

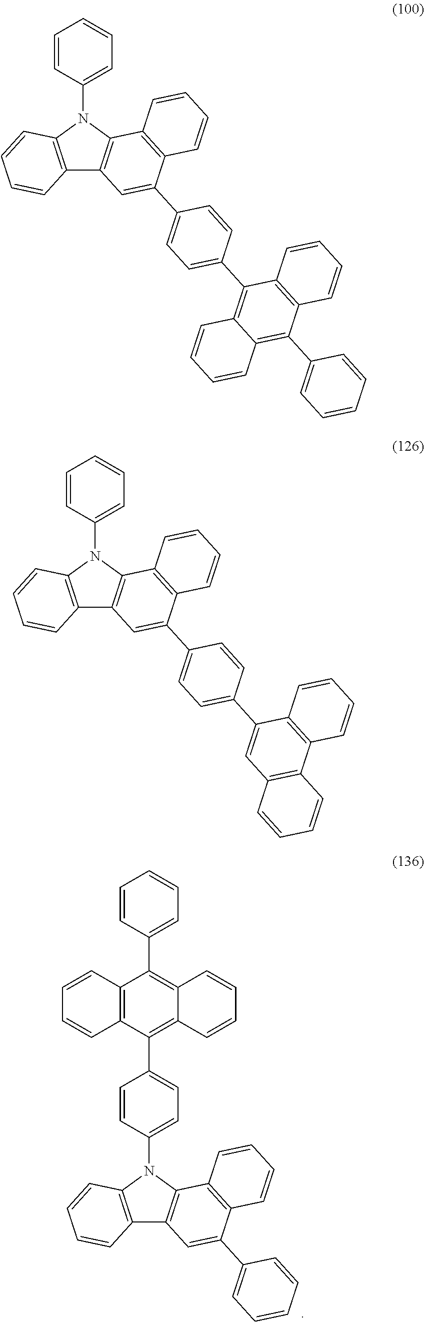 [SMILES: c1ccc(-c2c3ccccc3c(-c3ccc(-c4cc5c6ccccc6n(-c6ccccc6)c5c5ccccc45)cc3)c3ccccc23)cc1 ; c1ccc(-n2c3ccccc3c3cc(-c4ccc(-c5cc6ccccc6c6ccccc56)cc4)c4ccccc4c32)cc1 ; c1ccc(-c2c3ccccc3c(-c3ccc(-n4c5ccccc5c5cc(-c6ccccc6)c6ccccc6c54)cc3)c3ccccc23)cc1]

18. A light-emitting element comprising the organic compound according to claim 5.

19. A light-emitting element comprising:
the organic compound according to claim 5; and
a first organic compound comprising an anthracene skeleton.

20. A light-emitting element comprising:
an anode;
a cathode; and
a layer between the anode and the cathode, the layer comprising:
a first layer comprising a first organic compound comprising an anthracene skeleton; and
a second layer comprising the organic compound according to claim 5,
wherein the second layer is between the first layer and the anode.

21. A light-emitting element comprising:
an anode;
a cathode; and
a layer between the anode and the cathode, the layer comprising:
a first layer comprising a first organic compound comprising an anthracene skeleton and a carbazole skeleton; and
a second layer comprising the organic compound according to claim 5, wherein the second layer is between the first layer and the anode.

22. A light-emitting device comprising:
the light-emitting element according to claim 18; and
at least one of a transistor and a substrate.

23. An electronic device comprising:
the light-emitting device according to claim 22; and
at least one of a sensor, an operation button, a speaker, and a microphone.

24. A lighting device comprising:
the light-emitting device according to claim 22; and
a housing.
